(19) United States
(12) Patent Application Publication
(10) Pub. No.: US 2025/0041422 A1
(43) Pub. Date: Feb. 6, 2025

(54) METHOD OF TREATING ARRHYTHMIA, POPULATION OF PACEMAKER CARDIOMYOCYTES, AND METHOD OF TREATING CARDIAC ARRHYTHEMIA USING POPULATION OF PACEMAKER CARDIOMYOCYTES

(71) Applicant: OLIVIA SZU HSIEH LEE NAKAYA, Katy, TX (US)

(72) Inventor: Yu-Feng HU, Taipei City (TW)

(21) Appl. No.: 18/927,568

(22) Filed: Oct. 25, 2024

Related U.S. Application Data
(63) Continuation-in-part of application No. PCT/US2023/066203, filed on Apr. 25, 2023.
(60) Provisional application No. 63/334,691, filed on Apr. 26, 2022.

(51) Int. Cl. A61K 45/06

(52) U.S. Cl. CPC A61K 45/06

(57) ABSTRACT

A method of treating arrhythmia includes administering to a subject in need thereof an effective amount of a pharmaceutical composition including a therapeutic agent that increases the activity or expression of aldolase c (Aldoc) and a pharmaceutically acceptable carrier. The therapeutic agent modulates rhythmic activity of pacemaker cardiomyocytes in the subject. A population of pacemaker cardiomyocytes and a method of treating a cardiac arrhythmia using the same are also provided.

### CROSS-REFERENCE TO RELATED APPLICATION

This application is a continuation-in-part of International Application No. PCT/US2023/066203, filed on Apr. 25, 2023, which claims the benefit of U.S. Provisional Patent Application No. 63/334,691, filed on Apr. 26, 2022. The aforesaid applications are incorporated by reference herein in their entirety.

### FIELD

The disclosure relates to a method of treating arrhythmia. The disclosure also relates to a population of pacemaker cardiomyocytes and a method of treating a cardiac arrhythmia using the same.

### BACKGROUND

The sinoatrial node (SAN) initiates electric impulses for every heartbeat to maintain life. Its dysfunction causes a slow heart rate, insufficient blood supply, and detrimental consequences such as cardiac arrest (Epstein et al., Circulation, 2013). In contrast to atrial or ventricular cardiac tissue, the SAN consists of a network of pacemaker cardiomyocytes (PCs) encased with abundant fibroblasts and a heterogeneous connective tissue microenvironment (Camelliti et al., Circ Res, 2004; Perde et al., Folia Morphol (Warsz), 2016; Bressan et al., Cell Rep, 2018; Bleeker et al., Circ Res, 1980). The microenvironment, through the integration of PCs, mesenchymal lineages (including fibroblasts), and extracellular matrix organization, is required for the rhythmic activity of SANs during embryogenesis (Bressan et al.). Failure of extracellular matrix organization and likely fibroblast integration results in electrical dysfunction of SANs (Bressan et al.). Alteration of the microenvironment underlies the pathogenesis of SAN disorders. Although the molecular mechanisms underlying the ability of individual PCs to generate rhythmic electrical impulses have been well studied (Cingolani et al., Nat Rev Cardiol, 2018; Dobrzynski et al., Circulation, 2007), the biological process behind the microenvironmental niche in SANs, especially fibroblast-PC interactions, remains poorly understood.

Although the mechanisms underlying cardiac arrhythmias associated with electrical dysfunction of SANs are complex and not fully understood, it has been established that glycolysis plays an important role as the source of ATP to maintain the electrochemical gradient across the cardiac cellular membrane. Potassium (K⁺), calcium (Ca²⁺), and sodium (Na⁺) gradients are all modulated by ATP that arises from glycolysis. Moreover, inhibition of glycolysis is arrhythmogenic, while glucose-insulin-potassium (GIK) infusions in the setting of ischemia are anti-arrhythmic.

While drug treatments are often effective against cardiac arrhythmias, drugs frequently have side effects and require the patient to remember to take them on a daily basis. Mild to moderate side effects associated with these drugs include drowsiness, dizziness, nausea, bradycardia, and low blood pressure, while more severe side effects include torsades des pointes (a form of ventricular tachycardia) and even sudden death. Further, these drugs can cause arrhythmias at increased dosages due to their toxic effects on cardiac conduction at these levels.

Accordingly, there is a need to develop safe and effective compositions for treating cardiac arrhythmias that overcome some of the existing problems associated with the current treatment.

### SUMMARY

The present disclosure is based, at least in part, on the unexpected discovery that fibroblasts induce metabolic reprogramming by upregulating aldolase c (Aldoc) in pacemaker cardiomyocytes (PCs) and inhibitors of the PI3K signaling pathway increase Aldoc expression, which leads to regulation of beating rates of pacemaker cardiomyocyte. These results indicate that agents capable of modulating Aldoc expression in PCs, e.g., PI3K inhibitors, would benefit treatment of cardiac arrhythmia.

Accordingly, one aspect of the present disclosure features a method of treating cardiac arrhythmia in a subject, the method comprising administering to the subject an effective amount of a pharmaceutical composition comprising a therapeutic agent that increases the activity or expression of Aldoc and a pharmaceutically acceptable carrier. The therapeutic agent modulates rhythmic activity of pacemaker cardiomyocytes (PCs) in the subject.

In some embodiments, the therapeutic agent comprises a phosphoinositide 3 kinase (PI3K) inhibitor, an IkappaB kinase (IKK) inhibitor, an integrin, a nucleic acid encoding an integrin, an integrin agonist, a p38/MAPK agonist, or a combination thereof. In some examples, the therapeutic agent is a PI3K inhibitor. Examples include, but are not limited to, PIK-75, Duvelisib, Alpelisib, Copanlisib, Idelalisib, Eganelisib, or a combination thereof. In other examples, the therapeutic agent is an IKK inhibitor. In one example, the IKK inhibitor is BMS-34551.

In some embodiments, the subject for treatment is a human patient having or suspected of having arrhythmia. In some examples, the cardiac arrhythmia is bradycardia arrhythmia, sick sinus syndrome (SSS), a sinoatrial node disease, a sinoatrial node dysfunction or cardiac conduction disease. In specific examples, the cardiac arrhythmia is a cardiac conduction disease, which is an atrioventricular block (AV block) or bundle block.

In some embodiments, the pharmaceutical composition is administered to the subject orally. In other embodiments, the pharmaceutical composition is administered to the subject via a parenteral route. Examples include, but are not limited to, intravenous injection, intraarterial injection, intraperitoneal injection, intrapleural injection, intracardiac injection, or intrapericardial injection.

In another aspect, the present disclosure provides a cellular co-culture system, comprising fibroblasts and pacemaker cardiomyocytes (PCs), wherein the PCs are induced by T-box transcription factor 18 (Tbx18). In some embodiments, the PCs are derived from human pluripotent stem cells.

Also provided herein is a tissue sheet, comprising an extracellular matrix loaded with fibroblasts and cardiomyocytes, wherein the cardiomyocytes are engineered to express T-box transcription factor 18 (Tbx18). In some embodiments, the ratio of the fibroblasts to the cardiomyocytes ranges from 1:5 to 1:15, optionally 1:10. In certain embodiments, the cardiomyocytes are derived from ventricular cardiomyocytes.

Any of the cellular co-culture systems and tissue sheets provided herein can be used for screening for therapeutic agents that can regulate rhythmicity of pacemaker cardiomyocytes. Accordingly, also provided is a method for identifying a compound capable of regulating rhythmicity of pacemaker cardiomyocytes, the method comprising: (i) incubating a cellular co-culture system or a tissue sheet as described herein in the presence of a candidate compound; (ii) measuring a level of aerobic glycolysis of the pacemaker cardiomyocytes in the cellular co-culture system or the tissue sheet; and (iii) identifying the candidate compound as a compound capable of regulating rhythmicity of pacemaker cardiomyocytes, when the level of aerobic glycolysis in the pacemaker cardiomyocytes is enhanced relative to the pacemaker cardiomyocytes cultured in the absence of the candidate compound. In some embodiments, step (ii) is performed by measuring an expression level of aldolase c in the pacemaker cardiomyocytes in the cellular co-culture system of step (i).

In another aspect, provided herein is a population of pacemaker cardiomyocytes. The pacemaker cardiomyocytes are induced by the T-box transcription factor 18 (Tbx18) and cultured in the presence of a therapeutic agent that increases the activity or expression of aldolase c (Aldoc) in the pacemaker cardiomyocytes. In one embodiment, the population of pacemaker cardiomyocytes are cultured in the presence of a phosphoinositide 3 kinase (PI3K) inhibitor, an IkappaB kinase (IKK) inhibitor, and integrin, a nucleic acid encoding an integrin, an integrin agonist, a p38/MAPK agonist, or a combination thereof. In some examples, the population of pacemaker cardiomyocytes are cultured in the presence of a PI3K inhibitor, e.g., PIK-75, Duvelisib, Alpelisib, Copanlisib, Idelalisib, Eganelisib, or a combination thereof. In other examples, the population of pacemaker cardiomyocytes are cultured in the presence of an IKK inhibitor, e.g., BMS-34551.

Also within the scope of the present disclosure is a method of treating a cardiac arrhythmia (e.g., those disclosed herein) in a subject, comprising administering to a subject in need thereof an effective amount of a population of pacemaker cardiomyocytes described herein. In some embodiments, the population of pacemaker cardiomyocytes is administered by intravenous infusion or transplantation to heart tissues of the subject.

Also provided herein are pharmaceutical compositions comprising any of the therapeutic agents disclosed herein or the population of PCs as also disclosed herein for use in treating cardiac arrhythmia. Further provided herein are uses of such pharmaceutical compositions or PCs for manufacturing a medicament for the intended therapeutic use.

The details of one or more embodiments of the invention are set forth in the description below. Other features or advantages of the present invention will be apparent from the following drawings and detailed description of several embodiments, and from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1K include diagrams showing data related to Aldolase c-driven glycolysis regulation and pacemaker rhythmicity. FIG. 1A shows a heatmap of differential gene expression between Tbx18 (T-box transcription factor 18) pacemaker cardiomyocytes (PCs) and control ventricular cardiomyocytes (VMs). FIG. 1B shows a list of canonical pathways. FIG. 1C shows the expression levels of the key metabolic genes in glycolysis between Tbx18-PCs and control-VMs. The upper panel shows the key metabolic genes and metabolites in the glycolysis process. In the lower panel, the enzymes involved in glycolysis mostly increased, and only Aldoc was downregulated. A-C, n=4 for each group. *P<5.0×10⁻². FIG. 1D shows the expression of metabolic genes in glycolysis determined by real-time PCR. (Hexokinase-1 [Hk1], control-VMs, n=7; Tbx18-PCs, n=8; Hexokinase-2 [Hk2], control-VMs, n=5; Tbx18-PCs, n=7; Aldolase a and Aldolase c, control-VMs, n=7; Tbx18-PCs, n=12). FIG. 1E shows the Seahorse glycolysis stress test. Left panel: representative curves from the experiments. Glycolysis was persistently lower in Tbx18-PCs than in control-VMs. The right panel shows the measurements including basal glycolysis, % proton efflux rate (PER) and compensatory glycolysis. control-VMs, n=18; Tbx18-PCs, n=14. FIG. 1F shows the levels of glycolysis metabolites determined by mass spectrometry. The highest decrease was observed in the relative levels of G3P and DHAP to those of control-VMs. FIG. 1G shows the reduction ratio of metabolites in Tbx18-PCs with reference to control-VMs. The reduction ratio (%)=100*(Tbx18-PC metabolites−control-VM metabolites/control-VM metabolites). The reduction in DHAP and G3P levels reached a nadir in comparison to the other metabolites. FIGS. 1E-1F show control-VMs, n=3; Tbx18-PCs, n=4. FIG. 1H shows lactate levels in PCs determined by a colorimetric assay. n=7 for each group. FIG. 1I shows modulation of glycolysis changed pacemaker phenotypes, including Hcn4 expression and beating rate. Supplementation of active glycolysis metabolites with sodium pyruvate (1 mM) increased Hcn4 expression (Tbx18-PCs, n=10; Tbx18-PCs+sodium pyruvate, n=7; Tbx18-PCs+2-DG, n=11,) and beating rates (Tbx18-PCs, n=7; Tbx18-PCs+sodium pyruvate, n=8; Tbx18-PCs+2-DG, n=7). 2-DG inhibits glycolysis by competitively inhibiting the production of glucose-6-phosphate from glucose at the phosphoglucoisomerase level. Treatment with 2-DG (5 mM) decreased Hcn4 levels and beating rate. FIG. 1J shows adenoviral vector-mediated overexpression of Aldoc in Tbx18-PCs (Tbx18-PCs-control, n=8; Tbx18-PCs-Aldoc, n=8). FIG. 1K shows Aldoc overexpression increased the electrical firing rate (Tbx18-PCs-control, n=17; Tbx18-PCs-Aldoc, n=19) and Hcn4 expression (n=8 for each group). Representative tracing of the electrical firing rate in the Aldoc-overexpression and control vector groups is shown in the left panel. P-value determined by a two-tailed t test for all except FIG. 1I, determined by one-way ANOVA.

FIGS. 2A-21 include diagrams showing data related to fibroblast driven aldolase c-mediated glycolysis adaptation in pacemaker cardiomyocytes. FIG. 2A shows a glycolysis stress test in Tbx18-PCs and cocultures. Coculture with fibroblasts improved glycolysis, in comparison to the single culture of Tbx18-PCs. The left panel shows representative curves from the Seahorse glycolysis test for Tbx18-PCs and cocultures (Tbx18-PCs, n=10; coculture, n=12). FIG. 2B shows a mitochondrial stress test in Tbx18-PCs and cocultures. The left panel shows the representative curves from the mitochondrial stress test. Measurements of basal respiration, spare respiratory capacity, proton leak and ATP production did not differ between the two groups. Tbx18-PCs, n=11; coculture, n=12. OCR: oxygen consumption rate. FIG. 2C shows the levels in glycolysis metabolites determined by mass spectrometry were compared between Tbx18-PCs and cocultures (relative expression to control-VMs). After cocultures, most of the metabolites increased. However, G3P and DHAP levels exhibited the greatest changes (more than 2-fold, red arrow). *P<5.0×10⁻², Tbx18-PCs vs. cocultures; Tbx18-PCs, n=4; coculture, n=3. FIG. 2D shows the level of DHAP determined by ELISA in Tbx18-cocultures was higher than that in Tbx18-PCs (Tbx18-PCs, n=10; coculture, n=11). FIG. 2E shows Aldolase c transcripts increased in the cocultures with fibroblasts compared to the level in Tbx18-PCs alone (Tbx18-PCs, n=12; coculture, n=13). FIG. 2F shows coculture increased beating rates compared to those of single Tbx18-PC cultures (Tbx18-PCs, n=26; coculture, n=37). FIG. 2G shows coculture also increased the expression of distinct pacemaker genes (Hcn4, Cx45) compared to Tbx18-PCs alone (Tbx18-PCs, n=9; coculture, n=10). FIG. 2H shows the level of the end-product of glycolysis (lactate) represents the active status of glycolysis. The separate coculture of Tbx18-PCs and Tbx18-transduced fibroblasts was not associated with an increased level of lactate. Only contact-coculture containing both Tbx18-PCs and fibroblasts was associated with increased lactate levels. P-value determined by one-way ANOVA with an LSD post hoc test. FIG. 21 shows representative western blot images of ALDOC expression. No ALDOC expression was noted in fibroblasts with GAPDH as a protein control (n=4). However, ALDOC expression was observed in neonatal cardiomyocytes (VMs, n=4). P-value determined by Mann-Whitney test (FIG. 2A) or a two-tailed t-test (FIGS. 2B-2G).

FIGS. 3A-3M include diagrams showing data related to the fibroblast-pacemaker interaction to regulate intrinsic expression of aldolase c in pacemaker cardiomyocytes. FIG. 3A shows a heatmap of differential gene expression in cardiomyocytes isolated from single Tbx18 (T-box transcription factor 18) pacemaker cardiomyocyte (PC) cultures and fibroblast cocultures (Tbx18-PCs, single culture, n=4; coculture, n=3). FIG. 3B shows transcript levels of the key enzymes involved in glycolysis between cocultures and single cultures of Tbx18-PCs. *P<5.0×10⁻². The expression of Aldoc was increased. FIG. 3C shows the canonical pathways determined by IPA analysis. The detailed genes within these pathways, as shown in Table 6, revealed critical regulation of ECM receptors and their downstream signals after coculture. FIG. 3D shows the transcript levels of ECM-binding receptors. Compared to the other integrin subunits, ltgb1 levels were the most abundant and significantly increased after coculture (Tbx18-PCs, single culture, n=4; coculture, n=3). FIG. 3E shows Aldoc expression in Tbx18-PC cocultures decreased after treatment with the ltgb1 inhibitory antibody (IgG control, n=10; ltgb1 antibody, n=11). FIG. 3F shows a representative western blot of total and phosphorylated AKT showing no difference between Tbx18-PC single cultures and cocultures. FIG. 3G shows Aldoc expression after treatment with a PI3K inhibitor (Wortmannin, n=5 for each group). FIG. 3H shows a representative western blot of total and phosphorylated ERK and p38-MAPK showing increased total and phosphorylated p38-MAPK after coculture with fibroblasts. FIG. 31 shows reduced Aldoc expression after treatment with a p38-MAPK inhibitor (control, n=9; SB203580, n=9). FIG. 3J shows representative western blot of total and phosphorylated Rb and E2F1 showing increased total and phosphorylated Rb and E2F1 after coculture with fibroblasts. FIG. 3K shows a promoter binding site prediction for aldolase c determined using AnimalTFDB version 3.0 (bioinfo.life.hust.edu.cn/AnimaITFDB/). The prediction showed three E2F1 binding sites within the promotor of Aldoc. FIG. 3L shows knockdown of p38-MAPK, Rb, and E2F1 downregulated Aldoc expression (nontarget, n=13; Rb siRNA, n=12; E2F1 siRNA, n=13; p38-MAPK siRNA, n=13). Treatment with p38-MAPK, Rb, and E2F1 siRNAs reduced the expression of the corresponding target genes, as shown in FIGS. 23A-23C. FIG. 3M shows intraperitoneal injection of the ltgb1 inhibitory antibody decreased Aldoc expression in mouse SANs (IgG control, n=6; ltgb1 antibody, n=8). All P-values determined by a two-tailed t-test. Statistical analyses of western blotting results (F, H and J) and uncut blot data are provided in FIGS. 17A-22.

FIGS. 4A-4G include diagrams showing data related to engineered Tbx18-pacemaker tissue sheets recapitulate Aldoc-driven rhythmic machinery. FIG. 4A shows immunofluorescence staining of a Tbx18-PC tissue sheet. Cardiomyocytes are interspersed with fibroblasts (n=4, thickness of 28.3±10.9 μm). The surface of the engineered Tbx18-PC tissue sheets was mostly covered by fibroblasts. FIG. 4B shows electrical recordings of spontaneous firing in Tbx18-PC and control tissue sheets determined by MEA (control, n=8; Tbx18-PC tissue sheet, n=7). FIG. 4C shows the autonomic response of Tbx18-PC tissue sheets. Treatment with the sympathomimetic drug (epinephrine, 0.15 μg/mL, agonist of alpha and beta receptors) increased beating rates in Tbx18-PC tissue sheets compared to rates in controls. There were minimal changes in the beating rate in the control tissue sheets (control, n=6; Tbx18-PC tissue sheet, n=6). FIG. 4D shows fluorescence staining of HCN4 in Tbx18-PC tissue sheets. Abundant PCs with distinct HCN4 expressions could be observed within tissue sheets. No HCN4 expression was observed in controls (FIG. 24). FIG. 4E shows Hcn4 and Cx45 transcripts increased in Tbx18-PC tissue sheets compared to levels in controls. Hcn4, control, n=8; Tbx18-PC tissue sheets, n=5; Cx45, control, n=8; Tbx18-PC tissue sheets, n=7. FIG. 4F shows Aldoc expression was higher in Tbx18-PC tissue sheets than in controls (n=4 for both groups). FIG. 4G shows knockdown of Aldoc by siRNAs in Tbx18-PC tissue sheets downregulated Aldoc transcripts (FIG. 26) and decreased the electrical firing rate of PC tissue sheets (nontarget siRNA, n=11; Aldoc siRNA, n=10). The left panel shows a representative MEA tracing of nontarget siRNA and Aldoc siRNAs on Tbx18-PC tissue sheets. P-value determined by a two-tailed t-test (FIGS. 4B-4G).

FIGS. 5A-5F include diagrams showing data related to the regulation of in vivo pacemaker rhythms by aldolase c in vertebrates. FIG. 5A shows Aldoc expression in adult rat SANs determined by immunofluorescence staining. The PCs within SANs had co-expressions of HCN4 and Aldoc. Aldoc expression was observed in adult rat SANs (animal=3) but not in adult rat ventricles (animal=2). FIG. 5B shows Aldoc transcripts determined by real-time PCR, suggesting dominant expression of Aldoc in the SAN but not in atrial or ventricular tissues. SAN, n=9; atrium, n=4; ventricle, n=8. P-value determined by one-way ANOVA with an LSD post hoc test. FIGS. 5C-5D show in vivo Aldoc expression in the mouse SAN after transduction with AAV9-Aldoc siRNAs. Regional Aldoc expression within the mouse SAN was best evaluated by immunofluorescence staining, as the SAN could be precisely localized by the presence of HCN4 channels. A representative image is shown in FIG. 5C. The expressions of regional Aldoc decreased after the transduction of AAV9-Aldoc siRNAs, compared to scrambles. FIG. 5D shows the fluorescence intensity of Aldoc was significantly reduced after transduction with AAV9 siRNA (A.U.: arbitrary unit). P-value by a two-tailed t-test. Three animals were used for both groups. FIG. 5E shows representative ECG tracings of mice receiving AAV9-Aldoc or scramble siRNAs. In vivo Aldoc knockdown within SANs led to a slower heart rate compared to that in control mice. The intraperitoneal injection of epinephrine (2.5 μg, Epi) increased the heart rate in control mice but not in Aldoc knockdown mice. The results are provided in FIG. 5F. Scramble, n=8; Aldolase c knockdown, n=10. P-value by a repeated-measures ANOVA followed by an LSD post hoc test.

FIGS. 6A-6E include diagrams showing data related to Aldolase c regulating pacemaker activity in human induced pluripotent stem cell-derived cardiomyocytes. FIG. 6A shows immunofluorescence staining showing Aldoc expression in HCN4 (+) pacemaker cardiomyocytes among human IPS-CMs. Aldoc expression could not be observed in HCN4 (−) IPS-CMs. FIG. 6B shows Aldoc levels, calculated by fluorescence intensity, were higher in HCN4 (+) PCs than in HCN4 (−) cardiomyocytes (IPS-CMs, n=35; IPS-PCs, n=23). FIG. 6C shows Aldoc levels after transduction with adenoviral Aldoc vectors (control, n=4; Aldoc, n=6). FIG. 6D shows Aldoc overexpression in IPS-CMs after treatment with the adenoviral vector was associated with higher electrical firing rates in the MEA compared to rates in IPS-CMs transduced with control vectors, as shown in the representative MEA tracing. FIG. 6E shows that either at baseline (without epinephrine) or after epinephrine treatment (150 ng/mL), increased Aldoc expression in IPS-CMs drove a higher electrical firing rate than observed in controls; n=7 for both groups. P-value determined by a two-tailed t-test (FIGS. 6B-6C), or a repeated-measures ANOVA followed by an LSD post hoc test (FIG. 6E).

FIGS. 7A-7D include diagrams showing data related to the differential expressions of metabolic genes between Tbx18-pacemaker and control ventricular cardiomyocytes. FIG. 7A shows the regulatory genes of pyruvate oxidation. Pyruvate dehydrogenase (Pdh) complex is responsible for the pyruvate decarboxylation step and converts pyruvate (a product of glycolysis in the cytosol) to acetyl-CoA that links glycolysis to the TCA cycle. This complex includes Pdha1, Pdhb, Pdhx, Pdp, Dlat, and Dld. Pyruvate dehydrogenase kinase (Pdk1 to 4) inhibits the activity of the Pdh complex. FIGS. 7B-7D show metabolic genes in TCA cycle, pentose phosphate pathway, and fatty acid metabolism. These genes were either no changes or minimally increased. The pentose phosphate pathway generates NADPH and pentoses (5-carbon sugars) as well as ribose 5-phosphate, a precursor for the synthesis of nucleotides. n=4 for both groups. The *P-values for each comparison are as follow: Pdk1 (P=2.1×10⁻³), Pdp1 (P=2.7×10⁻²), Ppard (P=3.7×10⁻⁴) by a two-tailed t-test.

FIGS. 8A-8B include diagrams showing data related to the mitochondrial function in Tbx18-pacemaker cardiomyocytes. Mitochondria stress test (Seahorse XFp Cell Mito Stress Test) was used to measure mitochondrial function of Tbx18-PCs and control-VMs including basal respiration, spare respiratory capacity, proton leak, and ATP production. FIG. 8A shows the representative curves from the mitochondrial stress test. Only the spare respiratory capacity was reduced in Tbx18-PCs compared to controls (FIG. 8B). The other measurements, including basal respiration, proton leak, and ATP production, did not differ. *P=8.9×10⁻⁴ by a two-tailed t-test, control-VMs, n=14; Tbx18-PCs, n=16. OCR: oxygen consumption rate.

FIGS. 9A-9E include diagrams showing data related to metabolite levels among different metabolic pathways in Tbx18-pacemaker and control ventricular cardiomyocytes. Numerous metabolites in different metabolic pathways were analyzed for Tbx18-PCs and control-VMs, including those in the pentose phosphate pathway (FIG. 9A), TCA cycle (FIG. 9B), pyruvate oxidation (FIG. 9C), and energy molecules of ATP (FIG. 9D), GTP (FIG. 9D), and NADH (FIG. 9E). Overall, no significant difference could be observed between the two groups except the levels of Ribose 5-phosphate (R5P) and G3P. As transketolase and transaldolase convert two molecules of fructose 6-phosphate (F6P) and one molecule of G3P to three molecules of R5P, the reduction of R5P in the pentose phosphate pathway could be explained by the reduction of G3P due to Aldoc deficiency. R5P, *P=1.1×10⁻²; G3P, #P=5.5×10⁻². FIGS. 9A-9E: control-VMs, n=3; Tbx18-PCs, n=4. P-value by a two-tailed t-test.

FIGS. 10A-10E include diagrams showing data related to metabolite levels among different metabolic pathways in single cultures and cocultures of Tbx18-pacemaker cardiomyocytes. Metabolites among different metabolic pathways were analyzed for single cultures of Tbx18-PCs and cocultures with fibroblasts, including those in pentose phosphate pathway (FIG. 10A), TCA cycle (FIG. 10B), pyruvate oxidation (FIG. 10C), and energy molecules of ATP (FIG. 10D), GTP (FIG. 10D), and NADH (FIG. 10E). FIGS. 10A-10B show the levels of metabolites within the pentose phosphate pathway and TCA cycle were mostly marginally increased or not changed in cocultures compared to those in single cultures. FIG. 10C show pyruvate levels marginally increased after coculture, accompanied by marginal improvement of acetyl-CoA. FIG. 10D shows increased ATP levels in cocultures indicated the improvement of energy production, although other energy molecules were not changed, such as GTP or NADH (FIG. 10E). FIGS. 10A-10E: Tbx18-PCs, n=4; coculture, n=3. *P<5.0×10⁻² by a two-tailed t-test; R5P, *P=1.0×10⁻³; G3P, *P=2.4×10⁻².

FIGS. 11A-11C include diagrams showing data related to the regulation of calcium clock by aldolase c. FIG. 11A show the inhibition of Aldoc decreased LCRs in Tbx18-PC cocultures. The treatment of Aldoc siRNA on Tbx18-PC cocultures reduced the incidence of LCRs (nontarget controls vs. Aldoc siRNAs; 37%, n=54 cells vs. 5.7%, n=70 cells, *P=1.2×10⁻⁵ by Chi-square tests). The representative image of LCRs is shown in the right panel. LCR, indicated by white arrows, was observed in controls but not those after the treatment of siRNAs. LCR period: the time from the prior action potential-induced Ca²⁺ transient to the onset of LCR. FIG. 11B shows the rate of spontaneously oscillating Ca²⁺ transient (127.7±62.2 bpm, n=35 vs. 16.2±15.6 bpm, n=41, *P=1.5×10⁻¹² by a two-tailed t-test). FIG. 11C shows the linear correlation between the cycle length of calcium transients and LCR period (n=34 LCRs from 20 cells, by the linear regression analysis).

FIGS. 12A-12B include diagrams showing data related to protein expressions of aldolase c in fibroblasts. Representative full-length blots of ALDOC (FIG. 12A) and GAPDH (FIG. 12B) are shown. The box indicates representative blots of ALDOC and GAPDH in FIG. 21. The Aldoc expressions could be observed in neonatal ventricular cardiomyocytes (VMs) but not fibroblasts. The positive control was also shown after the transduction of the adenoviral human ALDOC vector (CMV promotor, 068583A, Applied Biological Materials, Richmond, BC, Canada).

FIGS. 13A-13C include diagrams showing data related to the different regulation of glycolysis in control ventricular cardiomyocytes and Tbx18-pacemaker cardiomyocytes after coculture with fibroblasts. FIG. 13A shows fibroblasts activated different regulation of metabolic genes within glycolysis in control-VMs and Tbx18-PCs after coculture with fibroblasts. Aldolase c transcripts were upregulated in Tbx18-PCs after the cocultures with fibroblasts. *P-value of aldolase c (Tbx18-PCs, n=15 vs. other three groups): control-VMs (n=10, P=2.7×10⁻³), control-coculture (n=14, P=2.5×10⁻²), or Tbx18-coculture (n=16, P=3.7×10⁻²). Instead, increased expression of aldolase a was observed in VMs after the coculture with fibroblasts. *P-value of aldolase a: control-coculture vs. Tbx18-PCs (P=9.7×10⁻³) or Tbx18-coculture (P=4.8×10⁻³). Aldolase a: n=12, 13, 15, and 15 for control-VMs, Tbx18-PCs, control-coculture, and Tbx18-coculture, respectively. Hk2 also marginally increased in control and Tbx18 cocultures, compared to control-VMs and Tbx18-PCs. *P-value of Hk2: Tbx18-PCs vs. control-coculture (P=4.3×10⁻²) or Tbx18-coculture (P=8.6×10⁻²). Hk2: n=9 for all four groups. Hk1: n=12, 13, 15, and 15 for control-VMs, Tbx18-PCs, control-coculture, and Tbx18-coculture, respectively. FIG. 13B shows increased catalyzing enzymes of glycolysis (aldolases) led to increased DHAP levels in Tbx18-PCs and control-VMs after the coculture with fibroblasts. *P-values vs. Tbx18-PCs: control-VMs (P=3.7×10⁻²), control-coculture (P=9.6×10⁻⁹), or Tbx18-coculture (P=7.5×10⁻⁹). #P-values vs. control-VMs: control-coculture (P=4.9×10⁻⁶) or Tbx18-coculture (P=3.4×10⁻⁶). n=12, 10, 12, and 11 for control-VMs, Tbx18-PCs, control-coculture, and Tbx18-coculture, respectively. FIG. 13C shows glycolysis activity in Tbx18-PCs and control-VMs, including basal and proton efflux rate by Seahorse functional assays, all improved after the coculture with fibroblasts. For basal glycolysis, *P-values vs. Tbx18-PCs: control-VMs (P=3.4×10⁻³), control-coculture (P=1.7×10⁻⁶), or Tbx18-coculture (P=5.2×10⁻⁶); #P-values vs. control-VMs: control-coculture (P=1.7×10⁻²), or Tbx18-coculture (P=2.7×10⁻²). For % PER from glycolysis (basal), *P-values vs. Tbx18-PCs: control-VMs (P=5.3×10⁻³), control-coculture (P=2.5×10⁻⁵), Tbx18-coculture (P=8.1×10⁻⁴). For compensatory glycolysis, *P-values vs. Tbx18-PCs: control-VMs (P=4.2×10⁻³), control-coculture (P=7.2×10⁻⁵), Tbx18-coculture (P=1.1×10⁻³). n=13, 10, 15, and 12 for control-VMs, Tbx18-PCs, control-coculture, and Tbx18-coculture, respectively. FIG. 13A-13C by one-way analysis of variance (ANOVA) with LSD post hoc test.

FIGS. 14A-14D show data related to cell sorting to isolate Tbx18-pacemaker cardiomyocytes. FIG. 14A show representative histogram of CD90 (+) fibroblasts. First, the fibroblasts stained with/without conjugated APC mouse anti-rat CD90 were used to set the threshold of positive expressions of CD90. Fluorescent intensity of more than 100 was considered positive for CD90. Therefore, the cells higher than this threshold were considered fibroblasts. FIG. 14B shows the cells from Tbx18-PC single and cocultures were selected first by forward scatter (FSC), and side scatter (SSC). FIG. 14C shows in the cocultures, CD90 (+) fibroblasts were defined by CD90 expression after a light scatter gate. Instead, those cells without CD90 expression, considered as cardiomyocytes, were collected (2.2±1.0×10⁵ cells) further for whole transcriptome analysis. FIG. 14D shows the collection of cardiomyocytes, CD90(−) cells, in the single cultures.

FIGS. 15A-15D include diagrams showing data related to differential expressions of metabolic genes between isolated pacemaker cardiomyocytes from single cultures and cocultures. The metabolic genes related to pyruvate oxidation (FIG. 15A), TCA cycle (FIG. 15B), pentose phosphate pathway (FIG. 15C), and fatty acid metabolism (FIG. 15D) were either not different or minimally changed. Tbx18-PCs, single culture, n=4; coculture, n=3. *P-value by a two-tailed t-test. Ogdh,*P=1.1×10⁻²; Rpia, *P=3.6×10⁻²; Slc25a20, *P=3.0×10⁻².

FIG. 16 is a diagram showing data related to the transcriptional changes of calcium clock-related genes in pacemaker cardiomyocytes after coculture with fibroblasts. The transcriptional expressions of calcium clock-related genes, including the relevant pathways related to metabolic/energetic regulation of automaticity, were analyzed from whole transcriptome expression (the PC cultures alone vs. isolated PCs from PC-fibroblast cocultures). No significant changes were observed in calcium channels (Cacna1c, Cacna1h, Cacnb2), SERCA2a (Atp2a2), calsequestrin (Casq2), ryanodine receptor (Ryr2), sodium/calcium exchanger 1 (Slc8a1), protein kinase A, or calcium/calmodulin-dependent protein kinase II (CamKII). Instead, Pde4a was the only gene with a differential change of more than two-fold and reached statistical significance (*P=3.7×10⁻², by a two-tailed t-test after correction for the multiple comparisons). Pde4a is predominantly responsible for cAMP degradation. The decrease of Pde4a in Tbx18-PCs after the coculture with fibroblasts might increase cytosolic cAMP levels. The increased cAMP levels activate protein kinase A or CaMKII-mediated phosphorylation of calcium channels and regulate the calcium clock within PCs. Tbx18-PCs, single culture, n=4; coculture, n=3.

FIGS. 17A-17E include diagrams showing data related to the integrin-mediated signal pathways after the coculture of pacemaker cardiomyocytes and fibroblasts. The integrin-mediated signal pathways were analyzed by western blot as shown from FIGS. 17A-17E. Compared to Tbx18-PC single cultures, these results suggested that the p38-MAPK-Rb-E2F1 pathway was activated after coculture of Tbx18-PCs with fibroblasts. FIG. 17A shows the expressions of total and phosphorylated AKT did not change between Tbx18-PCs from single cultures and cocultures. Tbx18-PCs, n=6; cocultures, n=7. FIG. 17B shows the expressions of total ERK increased in cocultures, as compared to single cocultures (*P=2.2×10⁻⁴). However, the expressions of phosphorylated ERK (p-ERK/tubulin) did not differ between groups. Rather, normalized p-ERK (p-ERK/total ERK) decreased in coculture, compared to single coculture (*P=1.0×10⁻²). FIG. 17C shows the expressions of total p38-MAPK (*P=3.3×10⁻³) and phosphorylated p38-MAPK (p-p38-MAPK/tubulin, *P=1.1×10⁻²) increased in cocultures, compared to single cultures. FIG. 17D shows the total Rb expressions increased in cocultures (*P=4.3×10⁻² vs. single cultures). The phosphorylated Rb (p-Rb/tubulin) marginally increased in cocultures (P=8.7×10⁻² vs. single cultures). FIG. 17E shows the E2F1 expressions increased in cocultures (*P=2.9×10⁻² vs. single cultures). FIGS. 17B-17E, n=4 for both groups. *P-values by a two-tailed t-test. The original blots are shown in the following FIGS. 18-22.

FIG. 18 is a diagram showing representative full-length blots of total and phosphorylated AKT. The box indicates representative western blots of AKT, p-AKT, and GAPDH in FIG. 3F.

FIG. 19 is a diagram showing representative full-length blots of total and phosphorylated ERK. The box indicates representative western blots of ERK, p-ERK, and tubulin in FIG. 3H.

FIG. 20 is a diagram showing representative full-length blots of total and phosphorylated p38-MAPK. The box indicates representative western blots of p38-MAPK, phospho-p38-MAPK, and tubulin in FIG. 3H.

FIG. 21 is a diagram showing representative full-length blots of total and phosphorylated Rb. The box indicates representative western blots of Rb, phospho-Rb, and tubulin in FIG. 3J.

FIG. 22 is a diagram showing representative full-length blots of E2F1. The box indicates representative western blots of E2F1 and tubulin in FIG. 3J.

FIGS. 23A-23C is a diagram showing data related to gene expressions in cocultures after the treatment of siRNAs. The expressions of p38-MAPK (FIG. 23A, *P=2.8×10⁻³), Rb (FIG. 23B, *P=2.7×10⁻²), and E2F1 (FIG. 23C, *P=2.1×10⁻³) were successfully reduced by siRNAs, as compared to nontarget siRNA. n=5 for nontarget and siRNAs. *P-value by a two-tailed t-test.

FIG. 24 is a diagram showing data related to HCN4 expressions in control tissue sheets. By immunofluorescent staining, pacemaker cardiomyocyte-specific ion channel (HCN4) was not detectable in control tissue sheets (n=4).

FIG. 25 is a diagram showing data related to Cx45 expressions in Tbx18-PC tissue sheets. By immunofluorescent staining of the Tbx18-PC tissue sheet, connexin45 (Cx45) expression was observed at the junction of cardiomyocytes.

FIG. 26 is a diagram showing data related to Aldolase c expression in Tbx18-pacemaker tissue sheets after the treatment of Aldoc siRNAs. The expressions of Aldoc in the tissue sheet were successfully reduced by siRNAs, as compared to nontarget siRNAs. n=4 for nontarget and Aldoc siRNA, both from 4 biologically independent experiments. *P=2.7×10⁻² by a two-tailed t-test.

FIG. 27A-27B include diagrams showing data related to the efficiency of Aldoc interference by AAV9-Aldoc siRNAs in the in-vitro mice cardiomyocyte models. FIG. 27A shows Aldoc expression could be observed in HL-1 cardiomyocytes by western blot. FIG. 27B shows the transduction of AAV9 Aldoc siRNAs significantly decreased Aldoc expressions, as compared to those from AAV9 scramble siRNAs. (scramble AAV siRNAs, n=5; Aldoc siRNAs, n=6. *P=4.0×10⁻² by a two-tailed t-test). The cell model of mice cardiomyocytes (HL-1) was used to test the efficiency of gene interference by AAV9-Aldoc siRNAs before in-vivo delivery. HL-1 is an AT-1 mouse atrial cardiomyocyte (Claycomb et al., Proc Natl Acad Sci USA, 1998). Cells were cultured in gelatin (214340, Becton Dickinson Biosciences)/fibronectin (F-1141, Sigma-Aldrich) coated T25 flasks. The cells were maintained in Claycomb medium (51800C, Sigma-Aldrich) with the following components: 0.1 mM Norepinephrine, 2 mM L-Glutamine, 100 U/ml Penicillin/Streptomycin and 10% Fetal bovine serum. One day after seeding, HL-1 cells were transduced with AAV9 scramble or Aldoc siRNA virus. Two to 3 days after transduction, Aldoc transcripts were analyzed by real-time quantitative PCR.

FIG. 28 is a diagram showing the transduction efficiency of AAV9-Aldoc siRNAs in mouse SANs. AAV9-Aldoc siRNAs were tagged with GFP proteins. Therefore, transduction efficiency could be analyzed by the presence of GFP. As shown in the figures, GFP expression was observed in 79.8±13.9% of cardiomyocytes over the SAN area. The transduction efficiency in mouse SAN was fair (6 animals).

FIG. 29 is a graph showing the RNA levels of gene transcripts of PI3K in biomaterial-converted pacemaker cardiocytes (P<0.05).

FIG. 30 is a graph showing RNA levels of PI3K in Tbx18-converted pacemaker cardiomyocytes (P<0.05).

FIGS. 31A-31B include graphs showing the relative Aldoc C expression (FIG. 31A) and beating rate in pacemaker cardiomyocytes (FIG. 31B) (determined by a 2-tailed t-test).

FIGS. 32A-32B are graphs showing the relative Aldoc C expression (FIG. 32A) and beating rate (FIG. 32B) after treatment of PIK-75 in pacemaker cardiomyocytes (P<0.05 vs. control by one-way ANOVA).

FIGS. 33A-33C are graphs showing the fold change of Aldoc (FIG. 33A), Hcn4 mRNA expression (FIG. 33B) and beating rate (FIG. 33C) after treatment of IKK inhibitor in pacemaker cardiomyocytes.

### DETAILED DESCRIPTION

The present disclosure is, in part, based on the unexpected discovery that fibroblasts induce metabolic reprogramming by upregulating aldolase c (Aldoc) in pacemaker cardiomyocytes (PCs) through integrin-dependent MAPK-E2F1 signals. This resulted in enhanced aerobic glycolysis and establishment of rhythmicity in the PCs. Aldoc upregulation in PCs was additionally obtained following treatment of cells with phosphoinositide 3 kinase (PI3K) and IkappaB kinase (IKK) inhibitors. Therefore, Aldoc-driven energy replenishment provides a basis for restoration of SAN dysfunction in subject with cardiac arrhythmic disorders.

### 1. Compositions and Methods for Treating Cardiac Arrhythmias

In some aspects, the present disclosure provides therapeutic agents, pharmaceutical compositions comprising such, and methods of using such for treating or alleviating symptoms of cardiac arrhythmias.

In some instances, the therapeutic agents may be compounds capable of driving metabolic reprogramming (e.g., activating or maintaining glycolysis) in pacemaker cardiomyocytes (PCs). As reported herein, glycolysis metabolism can regulate rhythmicity in pacemaker cardiomyocytes. In some embodiments, the therapeutic agents are activators for aldolase c (Aldoc), which enhances activity of Aldoc or expression levels of Aldoc in cardiomyocytes. Alternatively or in addition, the therapeutic agents are compounds that modulating the integrin-mediated signaling pathway. For example, the therapeutic agents may be an integrin or a nucleic acid encoding the integrin, or an integrin agonist. In other examples, the therapeutic agents may modulate downstream components of the integrin-mediated signaling pathway. In some examples, the therapeutic agents may be phosphoinositide 3 kinase (PI3K) inhibitors. In other examples, the therapeutic agents may be IkappaB kinase (IKK) inhibitors. In yet other examples, the therapeutic agents may be p38/MAPK agonists.

In other instances, the therapeutic agents may be a population of pacemaker cardiomyocytes, which may be treated by any one of the compounds capable of driving metabolic reprogramming as disclosed herein.

### (A) Aldolase c Activating Agents

In some embodiments, the therapeutic agents for use in any of the treatment methods may be aldolase c (Aldoc) activators. In some instances, the Aldoc activators may enhance Aldoc activity. In other instances, the Aldoc activators may enhance expression of Aldoc in cardiomyocytes.

The Aldoc activating agents as disclosed herein can be any biological agent capable of providing increased Aldoc activity, expression. In some embodiments, the therapeutic agent is a phosphoinositide 3 kinase (PI3K) inhibitor. In some embodiments, the therapeutic agent is an IkappaB kinase (IKK) inhibitor. In some embodiments, the therapeutic agent is an integrin. In some embodiments, the therapeutic agent is a nucleic acid encoding an integrin or aldolase c. In some embodiments, the therapeutic agent is an integrin agonist. In some embodiment, the therapeutic agent is a p38/MAPK agonist. In some embodiments, a combination of the foregoing therapeutic agents is included in the pharmaceutical composition.

In some embodiments, the aldolase c activation agent in the pharmaceutical composition is a PI3K inhibitor. In one embodiment, the PI3K inhibitor is PIK-75. In another embodiment, the PI3K inhibitor is Duvelisib. In another embodiment, the PI3K inhibitor is Alpelisib. In another embodiment, the PI3K inhibitor is Copanlisib. In another embodiment, the PI3K inhibitor is Idelalisib. In another embodiment, the PI3K inhibitor is Eganelisib. In another embodiment, a combination of PI3K inhibitors, such as the foregoing PI3K inhibitors, is included in the pharmaceutical composition.

In some embodiments, the Aldoc-activating agent comprises an integrin or nucleic acid encoding an integrin. In certain embodiments, the integrin or integrin in the nucleic acid is integrin α1, integrin α5, integrin α5, integrin β1, or a combination thereof, such as integrin α5 and integrin β1.

### (B) Pacemaker Cardiomyocytes (PCs)

In some embodiments, the present disclosure provides a population of pacemaker cardiomyocytes (PCs) that are treated to express increased levels of aldolase c as compared with naturally-occurring PCs. Such PCs can be use in methods of treatment and drug screening, as further described below.

In some embodiments, the PCs are obtained from PCs grown in a culture medium comprising an Aldoc-activating agent. In some embodiments, the PCs grown in the culture medium are derived from human pluripotent stem cells. In some embodiments, the PCs are induced PCs (iPCs) genetically engineered to express one or more T-box (Tbx) transcription factors, such as Tbx18. Alternatively, the PCs may be produced by culturing cardiomyocytes (e.g., quiescent ventricular cardiomyocytes or precursor cells thereof) in the presence of one or more suitable factors such as Tbx18, which induces production of PCs.

In some embodiments, the PCs are cultured in the presence of T-box transcription factor 18 (Tbx18) and an Aldoc-activating agent, such as a PI3K or IKK. In some examples, the Aldoc-activating agent is a PI3K inhibitor. In certain embodiments, the PI3K inhibitor is PIK-75, Duvelisib, Alpelisib, Copanlisib, Idelalisib, Eganelisib, or a combination thereof. In some examples, the Aldoc-activating agent is an IKK inhibitor. In one embodiment, the IKK inhibitor is BMS-34551.

In some embodiments, the PCs in the culture medium are transiently transfected or stably transformed with an expression vector (e.g., replication-defective AAV9 or lentiviral vector, selectable plasmid vector etc.) expressing aldolase c, a T-box transcription factor (e.g., Tbx18) and/or an Aldoc-activating gene product (e.g., integrin α1, integrin α5, integrin α5, integrin β1, or a combination thereof, such as integrin α5 and integrin β1).

In some embodiments, the population of PCs treated with one or more of the Aldoc-activating agents described herein exhibit improved cellular characteristics characterized by e.g., enhanced rhythmic activity, increased Aldoc expression, or increased electrical firing. In some embodiments, the improved cellular characteristics are reflected in an improvement of about 10% or more, including, e.g., at least about 20%, at least about 30%, at least about 40%, at least about 50%, at least about 60%, at least about 70%, at least about 80%, at least about 90% or more, as compared to a population of PCs, which are not treated with the Aldoc-activating agent. In other embodiments, the improved cellular characteristics are reflected in an improvement of at least about 1.1-fold or more, including, e.g., at least about 2-fold at least about 3-fold, at least about 4-fold, at least about 5-fold, at least about 6-fold, at least about 7-fold, at least about 8-fold, at least about 9-fold, at least about 10-fold or more, as compared to untreated PCs.

### (C) Pharmaceutical Compositions

In some aspects, provided herein are pharmaceutical compositions comprising one or more of the therapeutic agents disclosed herein and one or more pharmaceutically acceptable carriers. Such pharmaceutical compositions can be used in the methods for treating arrhythmia as disclosed herein.

Generally, any of the therapeutic agents disclosed herein such as an Aldoc-activating agent or pacemaker cardiomyocytes (PCs) can be formulated for administration to a subject as a pharmaceutical composition, e.g., together with a pharmaceutically acceptable carrier, diluent or excipient. A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

A pharmaceutical composition comprising any of the Aldoc-activating agents or PCs described herein (e.g., 1, 2, 3 or more Aldoc-activating agents described herein) may be formulated for administration by any administration route known in the art, such as parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form.

In some embodiments, the pharmaceutical compositions or formulations are for parenteral administration, such as intravenous, intra-arterial, intra-muscular, subcutaneous, or intraperitoneal administration. Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition is suitably formulated for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the Aldoc-activating agents of the invention may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical composition is suitably formulated for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, the formulations can be pre-loaded in a unit-dose injection device, e.g., a syringe, for intravenous injection.

Pharmaceutical compositions comprising nucleic acids or expression vectors encoding an Aldoc-activating agent, such as aldolase c or an integrin, may be administered to a subject using any method known in the art, including e.g., viral vectors (including vaccinia, modified vaccinia, adenovirus, retrovirus, lentivirus, and adeno-associated viral (AAV) vectors) or liposomes administered according to any suitable method known in the art by routes of administration described herein.

### (D) Therapeutic Applications

In another aspect, the present disclosure provides a method for treating arrhythmia in a subject having or suspected of having a cardiac arrhythmia condition. In one embodiment, the method for treating arrythmia comprises administering to the subject in need thereof an effective amount of a pharmaceutical composition comprising a therapeutic agent described herein, which increases the activity or expression of Aldoc in combination with a pharmaceutically acceptable carrier such that the therapeutic agent modulates rhythmic activity of pacemaker cardiomyocytes in the subject.

In another embodiment, the method for treating arrythmia comprises administering to a subject in need thereof an effective amount of a population of pacemaker cardiomyocytes (PCs) obtained from PCs grown in a culture medium comprising an Aldoc-activating agent described herein. In some embodiments, the PCs are obtained from PCs grown in a culture medium comprising an Aldoc-activating agent and a T-box transcription factor (Tbx18). In some embodiments, the population of PCs are obtained from grown in the culture medium are induced PCs (iPCs) genetically transformed to constitutively express Tbx18. In some embodiments, the PCs grown in the culture medium are derived from human pluripotent stem cells, optionally genetically transformed to express Tbx18.

The subject to be treated by the methods described herein can be a human (i.e., a male or a female of any age group, for example, a pediatric subject (e.g., an infant, child, or an adolescent) or an adult subject (e.g., a young adult, a middle-aged adult, or a senior adult)). The subject may also include any non-human animals including, but not limited to a non-human mammal such as cynomolgus monkey or a rhesus monkey. In certain embodiments, the non-human animal is a mammal, a primate, a rodent, an avian, an equine, an ovine, a bovine, a caprine, a feline, or a canine. The non-human animal may be a male or a female at any stage of development. The non-human animal may be a transgenic animal or a genetically engineered animal. A “patient” refers to a human subject in need of treatment for a cardiac arrythmia disease, such as those described herein.

The subject, such as a human patient, may have a cardiac arrythmia condition, such bradycardia arrhythmia, sick sinus syndrome (SSS), a sinoatrial node disease, a sinoatrial node dysfunction (SND) or cardiac conduction disease. In some embodiments, the cardiac conduction disease is an atrioventricular block (AV block), such as a first-degree AV block, a Mobitz type I second-degree AV block, a Mobitz type II second-degree AV block, or a bundle block, such as right bundle branch block, left bundle branch block, or fascicular block. Patients or subjects with SSS and SND may include arrythmias characterized by one or more of the following:
(1) Abrupt, inappropriate, severe sinus bradycardia;
(2) Sinus pause or arrest where there are no atrial activities, which usually reflects a failure of P cells to generate the action potential;
(3) Sinoatrial (SA) exit block where signals to the upper heart chambers are slowed or blocked, causing pauses or skipped beats. This is caused by the failure of the T cells to transmit impulses;
(4) Tachy-brady syndrome, characterized by bradycardia alternating with paroxysmal supraventricular arrhythmias. This is most frequently associated with atrial fibrillation (AF) and results from abnormal automaticity and conduction within the atrial tissue, affecting at least 50% of patients with SND;
(5) Failure to resume sinus rhythm after cardioversion, manifesting as a prolonged sinus pause;
(6) Atrial fibrillation with slow ventricular response in the absence of AV node blocking agents, which is likely due to the simultaneous degeneration in the AV node. In these patients, spontaneous or therapeutic termination of AF (cardioversion) results in a sinus pause due to concomitant SND. The annual incidence of complete AV block ranges from (0% to 4.5% with a median of 0.6%; and
(7) Chronotropic incompetence is defined as inappropriate bradycardia in which the heart rate is within regular range at rest but doesn't increase as much as it should with physical activity, thereby resulting in an inability to meet the metabolic demands. This is estimated to occur in 20% to 60% of patients.

In addition to cardiac arrythmia conditions, in certain embodiments, the Aldoc-activating agents may be used to treat non-arrythmia-related bradycardia, characterized by a heart rate of <60 bpm.

To perform the methods described herein, an effective amount of a Aldoc-activating agent (e.g., those described herein) can be administered to a subject in need of the treatment via any suitable route of administration.

An “effective amount,” “effective dose,” or an “amount effective to”, as used herein, refers to an amount of a pharmaceutical composition comprising an Aldoc-activating agent, such as a PI3K or IKK inhibitor, or cell as described herein, that is effective in producing the desired therapeutic, ameliorative, inhibitory or preventative effect, and/or results in a desired clinical effect in a subject, such as increased aldolase c expression, increased rhythmic activity, increased heartbeat, and/or increased electrical firing in PCs of a subject after administration of one or more Aldoc-activating agents or transplantation or infusion of PCs engineered to express increased levels of aldolase c. When needed, the Aldoc-activating agents or the PCs may be locally delivered to heart tissues in the subject. Effective amounts vary, as recognized by those skilled in the art, depending on route of administration, excipient usage, and co-usage with other active agents.

In the case of treating the arrythmia disease or condition, the desired response is an improved cardiac profile (e.g., enhanced rhythmic activity, increased heartbeat, electronic firing etc.) and/or reversal in progression of the disease. This may involve slowing the progression of the disease temporarily, although more preferably, it involves reversing the disease or halting progression of the disease permanently. This can be monitored by routine methods known in the art and/or described in the Examples herein. In some instances, the desired response to treatment of the disease or condition also can be delaying the onset or even preventing the onset of the disease or condition.

Dosages of pharmaceutically active agents can be determined by methods known in the art, see, e.g., Remington, The Science and Practice of Pharmacy (21st Ed. 2005). Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of one or more characteristics associated with the disorder for treatment. Alternatively, sustained continuous release formulations of may be appropriate. Various formulations and devices for achieving sustained release are known in the art. In one example, dosages for an Aldoc-activating agent or cell as described herein may be determined empirically in individuals who have been given one or more administration(s) of the Aldoc-activating agent or cell as described herein. Individuals are given incremental dosages of the active agents in the pharmaceutical composition(s). To assess efficacy of the treatment, one or more indicator(s) associated with the disorder can be followed throughout the course of treatment.

In some embodiments, administration to a subject of an effective amount of a pharmaceutical composition described herein results in an improved cardiac profile characterized by e.g., enhanced rhythmic heart activity, increased Aldoc expression, increased heartbeat, or increased PC electrical firing in the subject. In some embodiments, the improved cardiac profile is reflected in at least one of the foregoing cardiac profile characteristics by at least about 10% or more, including, e.g., at least about 20%, at least about 30%, at least about 40%, at least about 50%, at least about 60%, at least about 70%, at least about 80%, at least about 90% or more, as compared to an untreated subject. In some embodiments, administration to a subject of an effective amount of the Aldoc-activating agent or cell described herein results in, e.g., increased Aldoc expression in PCs of subject by at least about 1.1-fold or more, including, e.g., at least about 2-fold at least about 3-fold, at least about 4-fold, at least about 5-fold, at least about 6-fold, at least about 7-fold, at least about 8-fold, at least about 9-fold, at least about 10-fold or more, as compared to untreated PC cells.

An effective dose of an Aldoc-activating agent described herein, such as a PI3K inhibitor for the methods described herein can be between 0.01 mg/kg and 150 mg/kg body weight, or between 10 mg/kg and 80 mg/kg, or between 20 mg/kg and 60 mg/kg. In some instances, an effective dose of an Aldoc-activating agent described herein can be about 0.01, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150 mg/kg, and any range or value therein. In some instances, the dose can be even lower, e.g., as low as 0.001, 0.0005, or 0.0001 mg/kg or lower, and any range or value therein. In some instances, the dose can be even higher, e.g., as high as 200, 250, 300, 350, 400, 450, 500, 1000, 5000 mg/kg or higher, and any range or value therein.

A physician in any event may determine the actual dosage which will be most suitable for any subject, which will vary with the age, weight and the particular disease or disorder to be treated or prevented.

The frequency of administration of a composition of this invention can be as frequent as necessary to impart a desired therapeutic effect. For example, in some instances, an effective dose of the pharmaceutical composition containing an Aldoc-activating agent or cell is administered to a subject every day, every 2 days, or every 3 days. In other instances, the pharmaceutical composition can be administered one, two, three, four or more times per day; one, two, three, four or more times a week; one, two, three, four or more times a month; one, two, three or four times a year, or as necessary to control the condition. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

In some embodiments, a population of pacemaker cardiomyocytes (PCs) are transplanted or infused in the subject. In some embodiments, the PCs for transplantation are derived from myocytes, such as ventricular myocytes (VMs). In some embodiments, the PCs are obtained from PCs grown in a culture medium comprising an Aldoc-activating agent. In some embodiments, the PCs grown in the culture medium are derived from human pluripotent stem cells (e.g., induced pluripotent stem cells which can be differentiated from e.g., PBMCs according to methods known in the art). In some embodiments, the PCs are induced PCs (iPCs) genetically engineered to express one or more T-box (Tbx) transcription factors, such as Tbx18.

The PCs for transplantation are generally cultured ex vivo prior to transplantation in a subject. In some embodiments, the PCs are isolated from the same subject (autologous), cultured ex vivo, and then transplanted back to the subject. Alternatively, the PCs can be allogenic, i.e., obtained from a different subject of the same species. For allogeneic PC transplantation, allogeneic PCs may have an HLA type that matches with the recipient.

In some embodiments, the subject can further receive a second transplantation of PCs after the transplantation of the first population of PCs. The second transplantation of PCs can be performed any time after the first PC transplantation. For example, the second PC transplantation can be performed about 3 days or longer, including 4 days, 5 days, 6 days, 7 days, 2 weeks, 3 weeks, 4 weeks, or longer, after the first PC transplantation.

In some embodiments, administration of Aldoc activating agents, such as PI3K or IKK inhibitors, or Aldoc expressing/activating cell compositions disclosed herein, is via direct cardiac injection (e.g., during electronic pacemaker implantation or explantation). In some embodiments, systemic injection is used. In some embodiments, intracoronary injection may be used. In some embodiments, catheter-directed administration may be used. In some embodiments, a map-guided catheter system (e.g., NOGA®) may be used in order to focally administer the compositions. Other mapping or guidance techniques may be used in some embodiments. For example, in some embodiments, fluoroscopy-based guidance may be employed. In some embodiments, electroanatomical guidance may be employed. Mapping of specific structures (including but not limited to the His Bundle, the right or left portions of the bundle, the Purkinje fibers, etc.) by intracardiac electrograms may be used in some embodiments. Moreover, X-rays or magnetic catheters may be also used in some embodiments to guide delivery of a catheter, needle, or other delivery device(s) to a desired target location.

In some embodiments, a focal delivery approach advantageously reduces the time to generation of an active biological pacemaker. In some embodiments, Aldoc-activating expression constructs in the transplanted PCs may employ constitutive promoters. In some embodiments, Aldoc-activating expression constructs may employ tissue-specific (or cell type-specific) regulatory elements to facilitate improved biological pacemaker function.

In some embodiments, delivery of therapeutic Aldoc-activating agents or cells is achieved by focal injection into the apex of the heart. In some embodiments, transduction is achieved by focal injection to the left ventricular apex. In some embodiments, a right-sided (e.g., right side of the heart, either atrium or ventricle) approach is used, in order to reduce the risk of stroke or other embolism. However, in some embodiments, left-sided approaches are used. In some embodiments, an injection catheter is introduced via the right atrium (rather than the right ventricle), in order to access the Bundle of His or AV node from above. In some embodiments, trans-septal catheter methods are used to introduce an injection catheter into the left atrium or left ventricle without the need for arterial access, thereby reducing stroke risk. In still some embodiments, the introduction of an injection catheter is by way of the cardiac veins via the sinus of Valsalva for injection of a biologic as disclosed herein into various targets of the ventricles. Such an approach is similar to that used for the placement of pacer leads in cardiac resynchronization therapy.

Thus, in certain embodiments, the pharmaceutical cell compositions disclosed herein can be delivered to either the right atrium, right ventricle, SA node, AV node, Bundle of His, and/or left and right bundle branches. Moreover, through cannulation of the coronary sinus and its venous branches delivery to multiple left ventricular sites is achieved in several embodiments. Advantageously, in those patients with unfavorable coronary venous anatomy, access to the left side is achieved, in several embodiments, from the right side through a trans-septal puncture which allows direct access to left sided structures without the need of arterial access.

In some embodiments, the administration methods may include administration of compounds to increase the microvascular permeability of the cardiac tissue. Suitable vascular permeability agents (administered prior to, during, or after administration of a gene transfer vector) include e.g., administration of solution having less than about 500 μM calcium, substance P, histamine, acetylcholine, an adenosine nucleotide, arachidonic acid, bradykinin, endothelin, endotoxin, interleukin-2, nitroglycerin, nitric oxide, nitroprusside, a leukotriene, an oxygen radical, phospholipase, platelet activating factor, protamine, serotonin, tumor necrosis factor, vascular endothelial growth factor, a venom, a vasoactive amine, or a nitric oxide synthase inhibitor, serotonin, vascular endothelial growth factor (VEGF), a functional VEGF fragment, or a combination thereof.

Any of the Aldoc-activating agents or Aldoc-activated PCs described herein may be used in conjunction with other agents, including conventional agents (e.g., other agents for treating the arrythmia disorder) that serve to enhance and/or complement the effectiveness of the agents.

### II. PC-Containing Systems and Uses Thereof

As previously mentioned, it was surprisingly discovered that fibroblasts induce metabolic reprogramming and activate PC-specific expression of aldolase c through integrin-dependent cell contact. Integrins are heterodimeric transmembrane cell adhesion molecules made up of alpha (a) and beta (13) subunits arranged in numerous dimeric pairings which heterodimeric integrins on the PCs. Exemplary integrin heterodimer subunits in PCs for binding to fibroblasts include integrins α1, α3, α5 (CD49e), α6 (CD49f), α7, α9, and α10, which may be paired with β1, β3 (CD61), or β5. Exemplary heterodimers expressed or induced on PCs by fibroblasts include α5β1, α1β1, and α7β1.

Thus, the present disclosure also provides PC-containing systems, such as cellular co-culture systems and tissue sheets as disclosed herein, and their uses, e.g., in drug screening.

### A. PC-Containing Systems

In another aspect, the present disclosure provides a cellular co-culture system, comprising fibroblasts and pacemaker cardiomyocytes (PCs) for preparing PCs for drug screening, cell therapy or experimental. In some embodiments, the PCs are derived from myocytes, such as ventricular myocytes (VMs). In some embodiments, the PCs have been genetically engineered (or stably transformed) to express one or more T-box transcription factors, such as Tbx18. In certain embodiments, the PCs are derived from VMs transduced with an adenoviral human Tbx18 (i.e., Tbx18-PC). In some embodiments, the PCs in the co-culture system are human induced pluripotent stem cells (IPS-CMs) derived from human pluripotent stem cells (e.g., collected from donor PBMCs). See Tsai, M H et al., Stem Cell Research, (2021) 54:102416; and See Chiu, Y T et al., Stem Cell Research, (2021) 54:102419. In some instances, the cardiomyocytes and fibroblasts in the co-culture system form a monolayer.

In some embodiments, the fibroblasts are isolated from myocytes, including VMs and atrial myocytes (AMs). Methods for isolating VMs are described in the Examples.

In some embodiments, the PCs in the co-culture system are grown in the presence of an Aldoc-activating agent described herein, such as a PI3K inhibitor (e.g., PIK-75, Duvelisib, Alpelisib, Copanlisib, Idelalisib, Eganelisib, or a combination thereof) or an IKK inhibitor (e.g., BMS-345541).

In some embodiments, the PCs in the co-culture system are stably transformed with one or more expression vectors expressing e.g., aldolase c and/or one or more integrins as described herein, such as α5, integrin β1, or a combination thereof. The expression vectors may be selected or designed to express these genes constitutively or under the control of a cardiomyocyte-specific promoter (e.g., rat ventricle-specific cardiac myosin light chain 2 (MLC-2v) promoter, murine α-MHC promoter, and a hybrid troponin2 (TNNT2)-cardiac alpha actin (ACTC)) promoter. See Griscelli, F. et al., C R Acad Sci III (February 1997), 320(2):103-12; Aikawa, R. et al., J. Biol. Chem. (May 24, 2002), 277(21):18979-85; Fiedorowicz, K. et al., Sci. Reports, (Feb. 5, 2020) 10, article number 1895.

In another aspect, the present disclosure provides an engineered Tbx18-PC tissue sheet mimicking the three-dimensional microenvironment and phenotypes of in vivo SANs. In contrast to the co-culture system characterized by cardiomyocytes and fibroblasts forming a monolayer, a 3D tissue sheet has an extracellular matrix (e.g., Matrigel®) with several layers of cardiomyocytes and fibroblasts piling up thereon. In accordance with the present disclosure, the engineered tissue sheet may be used to screen drugs for enhancing pacemaker rhythmicity, provide a source of therapeutic cells for treatment, and provide a means for further study of Aldoc-driven rhythmic machinery in PC cells, including e.g., the molecular bases for induction of Hcn4 expression, PC beating, and PC electrical firing.

In one embodiment, the Tbx18-PC tissue sheet is prepared by inducing the expression of Tbx18 in an engineered tissue constructed from a mixed culture of VMs and fibroblasts with Matrigel as described in the Examples. Compared with control tissues, the Tbx18-PC tissue sheet exhibits increased Aldoc expression and expression of PC-specific genes, including Hcn4 and Cx45.

The ratio of fibroblasts to VMs or PCs in the co-cultures or tissue sheets may be variable. In some embodiments, the ratio of fibroblasts to VMs is about 1:5, 1:10, 1:15 or 1:20. Similarly, the ratio of fibroblasts to PCs, such as Tbx18-PC, may be about 1:5, 1:10, 1:15 or 1:20. In some embodiments, the co-cultured cells are grown on plates seeded with collagens, fibronectins, laminins and/or Matrigel®.

### (B) Drug Screening

Also provided are methods for identifying compounds capable of regulating rhythmicity of pacemaker cardiomyocytes. In one embodiment, the method comprises (i) incubating a cellular co-culture system comprising fibroblasts and pacemaker cardiomyocytes (PCs) or a tissue sheet thereof in the presence of a candidate compound (e.g., as described in section IV); (ii) measuring a level of aerobic glycolysis of the PCs (such as Tbx18-CMs) in the cellular co-culture system; and (iii) determining whether the candidate compound is capable of regulating rhythmicity of PCs based on the results in step (ii). In some embodiments, levels of glycolysis metabolites (e.g., lactate, dihydroxyacetone phosphate (DHAP), glyceraldehyde-3-phosphate (G3P)) may be measured to determine comparative levels of glycolysis in different cell cultures (e.g., separate (PC, VM, fibroblast (FB) cultures, contact co-cultures (PC-FB, VM-FB) and separate co-cultures (two cell types separated by porous membrane) as described in the Examples.

In one embodiment, the candidate compound is determined to meet the foregoing identification criterium in step (iii) by measuring the level of aerobic glycolysis in the PCs and determining whether the level of aerobic glycolysis is enhanced relative to the level of aerobic metabolism measured in control PCs cultured in the absence of the candidate compound. In another embodiment, the candidate compound is determined to meet the foregoing identification criterium in step (iii) by measuring the expression level of aldolase c (Aldoc) in the PCs in the cellular co-culture system and determining whether the expression level of Aldoc is increased in PCs cultured in the presence of the candidate compound relative to the expression level of Aldoc in the control PCs cultured in the absence of the candidate compound. In another embodiment, the candidate compound is determined to meet the foregoing identification criterium in step (iii) by measuring the beating or electrical firing rate of the PCs in the cellular co-culture system and determining whether the beating or electrical firing rate is increased in the presence of the candidate compound relative to the beating or electrical firing rate in the control PCs cultured in the absence of the candidate compound.

In another embodiment, the method comprises (i) incubating a cellular co-culture system comprising fibroblasts and PCs (such as Tbx18-CMs) or a tissue sheet thereof in the presence of a candidate compound; (ii) incubating a cellular co-culture system comprising fibroblasts and ventricular cardiomyocytes (VMs) or a tissue sheet thereof in the presence of the candidate compound in step (i); (iii) measuring a level of aerobic glycolysis of the PCs and VMs in the co-culture systems in steps (i) and (ii), respectively; and (iv) determining whether the candidate compound is capable of regulating rhythmicity of the PCs in step (i), based on a comparison of the results in step (iii).

In one embodiment, the candidate compound is determined to meet the meet the foregoing identification criterium in step (iv) by measuring the expression levels of aldolase c in the PCs and VMs in the cellular co-culture systems in steps (i) and (ii), respectively; and determining whether the expression level of Aldoc is increased in the PCs relative to the VMs. In another embodiment, the candidate compound is determined to meet the foregoing identification criterium in step (iv) by measuring the beating or electrical firing rates of the PCs and VMs in the cellular co-culture systems in steps (i) and (ii), respectively; and determining whether the beating or electrical firing rate is increased in the PCs relative to the VMs.

In some embodiments, comparative measurements of the foregoing variables may be evaluated between different cell cultures (e.g., separate (PC, VM, fibroblast (FB) cultures, contact co-cultures (PC-FB, VM-FB) and separate co-cultures (two cell types separated by porous membrane) to identify candidate compounds, as described in the Examples. Methods for measuring expression levels of gene products, such as aldolase c, are well known in the art and are further described in the Examples. In some embodiments, comparative measurements between cell populations may be made without separating PMs, VMs, and fibroblasts from their co-cultures by e.g., immunofluorescent staining. In other embodiments, comparative measurements are carried out after separating the PMs, VMs, and/or fibroblasts from one another by e.g., fluorescence activated cell sorting (FACS), or other methods known in the art and further described in the Examples.

In some embodiments, the PCs in the foregoing screening methods are derived from myocytes, such as ventricular myocytes (VMs). In some embodiments, the PCs have been genetically engineered (or stably transformed) from VMs to express one or more T-box transcription factors, such as Tbx18. In certain embodiments, the PCs are derived from VMs transduced with an adenoviral human Tbx18 (i.e., Tbx18-PC) as described in the Examples. In some embodiments, the PCs in the co-culture system are human induced pluripotent stem cells (IPS-CMs) derived from human pluripotent stem cells as described herein, and optionally engineered to express Tbx18.

Candidate substances for screening according to the methods described herein include, but are not limited to, small (e.g., less than about 2000 Mw, less than about 1000 Mw, or less than about 800 Mw) organic compounds, inorganic molecules including but not limited to salts or metals fractions of tissues or cells, nucleic acids, polypeptides, siRNAs, antisense molecules, aptamers, ribozymes, triple helix compounds, or antibodies.

Candidate molecules may encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug like.” Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 angstroms to about 15 angstroms.

High throughput assays for the presence, absence, quantification, or other properties of nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (e.g., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, MA; Air Technical Industries, Mentor, OH; Beckman Instruments, Inc. Fullerton, CA; Precision Systems, Inc, Natick, MA; etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

### III. Kits for Use in Treating Arrhythmia-Related Cardiac Disorders

The present disclosure further provides kits for use in treating the cardiac arrythmia disorders described herein. Such kits can include containers containing one or more Aldoc-activating agents, cell formulations and/or components for preparing one or more Aldoc-activating formulations for therapeutic use. In some embodiments, the containers may include lyophilized Aldoc-activating agent or cell compositions and solutions for resuspending the lyophilized components for administration. The containers may be provided in unit doses, bulk packages (e.g., multi-dose packages) or sub-unit doses.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. Kits may optionally provide additional components, such as buffers and interpretive information. Normally, the kit comprises a container and a label or package insert(s) on or associated with the container. In some embodiments, the present disclosure provides articles of manufacture comprising contents of the kits described above.

In some embodiments, the kit can comprise instructions for use in accordance with any of the methods described herein. The instructions generally include information as to dosage, dosing schedule, and route of administration for the intended treatment. Instructions supplied in the kits are typically written instructions on a label or package insert (e.g., a paper sheet included in the kit), but machine-readable instructions (e.g., instructions carried on a magnetic or optical storage disk) are also acceptable. The included instructions can comprise a description of administration of the Aldoc-activating formulation to treat, delay the onset, or alleviate an arrythmia-related cardiac disorder according to any of the methods described herein. The kit may further comprise a description of selecting an individual suitable for treatment based on identifying whether that individual has, is suspected of having, or is at risk for the disorder.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985>>; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1 Fibroblasts Drive Metabolic Reprogramming in Pacemaker Cardiomyocytes

The sinoatrial node (SAN) initiates electric impulses for every heartbeat to maintain life. Its dysfunction causes a slow heart rate, insufficient blood supply, and detrimental consequences such as cardiac arrest. In contrast to atrial or ventricular cardiac tissue, the SAN consists of a network of pacemaker cardiomyocytes (PCs) encased with abundant fibroblasts and a heterogeneous connective tissue microenvironment. This unique structure of SANs is well conserved across vertebrate species. The microenvironment, through the integration of PCs, mesenchymal lineages (including fibroblasts), and extracellular matrix organization, is required for the rhythmic activity of SANs during embryogenesis. Failure of extracellular matrix organization and likely fibroblast integration results in electrical dysfunction of SANs. At the other extreme, extensive fibrosis of the SAN also leads to pacemaker failure. Alteration of the microenvironment underlies the pathogenesis of SAN disorders. Although the molecular mechanisms underlying the ability of individual PCs to generate rhythmic electrical impulses have been well studied, the biological process behind the microenvironmental niche in SANs, especially fibroblast-PC interactions, remains poorly understood (Cingolani et al., Nat Rev Cardiol, 2018; Dobrzynski et al., Circulation, 2007).

The SAN is tiny with a paucity of PCs. Operable cell or tissue models are either generally lacking or difficult to handle (Cingolani et al.). To study functional interactions within the microenvironment, recapitulation of the complex structure of the PCs and fibroblasts within the SAN is necessary, but such models have yet to be developed. This presents a barrier to studying the biological function of this critical tissue, and obtaining models of SAN diseases. Recently, induced pacemaker cardiomyocytes, generated by T-box transcription factor 18 (Tbx18) transduction or biomaterial, recapitulated not only electrical and morphological phenotypes, but also the metabolic properties of native SAN cardiomyocytes (Kapoor et al., Nat Biotechnol, 2013; Gu et al., Exp Mol Med, 2019; Hu et al., Nat Biomed Engineer). It is possible that Tbx18-induced PCs, considered a replacement for native PCs, might be used to establish engineered models to study unknown SAN biological processes, especially intercellular functional interactions within the microenvironment (Grijalva et al., Adv Sci (Weinh), 2019).

In this study, Tbx18-induced PCs and engineered tissue were utilized to explore how fibroblasts regulate the functional integrity of SANs. Fibroblasts drove PC-specific expression of aldolase c (Aldoc, an enzyme involved in glycolysis metabolism) through integrin-dependent cell contact. This machinery critically maintained intrinsic aerobic glycolysis in PCs and regulated pacemaker activities. Aldoc-mediated rhythmic activity was faithfully validated in an in vitro engineered model, in vivo in mice, and in human-induced pluripotent stem cell-derived cardiomyocytes. These findings highlight the importance of the SAN microenvironment in determining its energy metabolism and rhythmicity. Moreover, tissue engineered with Tbx18-induced PCs could be a feasible in vitro platform to study SAN physiology.

### Materials and Methods

### Neonatal Rat Ventricular Cardiomyocyte and Fibroblast Isolation

Neonatal rat ventricular cardiomyocytes (VMs) were isolated from 1-2-day-old Sprague-Dawley rat pups (laboratory animal center, National Yang Ming Chiao Tung University) as previously described (Kizana et al., Circ Res, 2007; Sekar et al., Circ Res, 2009). In brief, the lower one-third of the rat heart was cut to prevent atrioventricular nodal and His-Purkinje cells contamination. The VMs were isolated with 2.5% trypsin (15090046, no Phenol Red, Thermo Fisher Scientific [GIBCO®]), MA, USA) and collagenase (Type II, 17101015, GIBCO®), and resuspended in medium. After tissue lysis, the resuspended cells (fibroblasts and VMs) were seeded in a T150 culture flask (430824, Corning®, NY, USA) for 60 minutes (repeated twice) (Neuss et al., Cell Tissue Res, 1996). The nonadherent cells were mainly VMs, while attached cells were fibroblasts. The nonadherent cells were further seeded in 10% FBS medium for 2 days, and then the concentration of FBS (fetal bovine serum, SH30070.03, Cytiva [Hyclone], Washington, USA) in the medium was reduced to 2% for the experiment. The culture medium was based on M199 (11150059, Thermo Fisher Scientific) with the following components: 10 mM HEPES (14185052, Thermo Fisher Scientific), 0.1 mM non-essential amino acids (11140050, Thermo Fisher Scientific), 3.5 mg/mL glucose (G-7021, Sigma-Aldrich, MO, USA), 2 mM L-glutamine (A2916801, Thermo Fisher Scientific), 4 μg/ml vitamin B12 (V-2876, Sigma-Aldrich), 100 U/ml penicillin (15140122, Thermo Fisher Scientific) and FBS. For the collection of fibroblasts, 5 to 7 days until the confluence of the attached cells was reached, the fibroblasts were collected and stored with 10% dimethyl sulfoxide (DMSO/FBS, D2650, Sigma-Aldrich) in liquid nitrogen until use. Before the experiments of cells or engineered tissues, thawed fibroblasts were cultured until confluence, and then subcultures (1-2 passages) were used for the experiments.

### Cell Model of Tbx18-Induced Pacemaker Cardiomyocytes

The Tbx18-PCs were used as the cell model of pacemaker cardiomyocytes, which were generated by the transduction of VMs with adenoviral human Tbx18 (MOI [multiplicity of infection]: 10; green fluorescent protein [GFP] reporter, ADV-225152, Vector Biolabs, Malvern, USA) for 24 hours (Kapoor et al; Hu et al.). Those with adenovirus-CMV-GFP were used as control VMs (Tsai et al., J Biomed Sci, 2015). For the cocultures of Tbx18-PCs and fibroblasts, a ratio of fibroblasts/VMs ( 1/10) was first plated on fibronectin-coated wells, and adenoviral Tbx18 was transduced for 24 hours one day after seeding to induce PCs. The constant beating was usually observed 3-4 days after Tbx18 adenovirus transduction, suggesting successful conversion (Tbx18-PCs). Therefore, the beating rates, metabolic and molecular phenotypes, including microelectrode array (MEA), whole transcriptomes, Seahorse analysis, metabolomics, glycolysis metabolites (lactate or DHAP), protein and gene transcripts were analyzed 3-5 days after Tbx18 transduction. The beating rates were recorded by MEA or video. An inverted microscope (AXIO Observer Al, Carl Zeiss AG, Oberkochen, Germany) was used for 10-second video capture. Video was analyzed with Q Capture Pro 6.0 (Teledyne Technologies, CA, USA).

For the experiments of drug treatment, Tbx18-PCs after re-expression of Tbx18 were treated with sodium pyruvate (1 mM, 11360-070, Thermo Fisher Scientific, 3 days), 2-deoxy-D-glucose (2-DG, 5 mM, D8375, Sigma-Aldrich, 3 days), wortmannin (PI3K inhibitor, 100 nM, 12-338, Merck Millipore, MA, USA, 4 days), anti-integrin β1 monoclonal antibody (ltgb1, MAB1987Z, 10 μg/mL, Merck Millipore, 2 days), or SB203580 (p38-MAP kinase inhibitor, 559389, 1 μM, Merck Millipore, 3 days), respectively. Then the beating rate and gene expression were analyzed.

### Separate Culture of Fibroblast and VMs

VMs were seeded on a 48-well culture plate (1.82×105 cells), and a 6.5 mm Transwell® with 0.4 μm Pore Polyester Membrane Insert (3470-clear, Corning®) was placed inside the culture plate. Fibroblasts (1.82×10⁴ cells) were seeded on the membrane in the transwell to avoid physical contact with VMs. The final composition of fibroblasts with VMs was 1:10, similar to contact-cocultures of Tbx18-PCs and fibroblasts. Adenoviral human Tbx18 cells were transduced for 24 hours, as previously mentioned. Three to four days after transduction, the medium was collected to analyze lactate levels.

### Measurement of Lactate Concentration

Culture medium from Tbx18-PCs, contact-coculture, separate-coculture, and Tbx18-fibroblast were collected to measure lactate concentration (A95550, Beckman Coulter, Brea, CA, USA). Lactate was converted to pyruvate by lactate oxidase in the presence of hydrogen peroxide (H₂O₂). H₂O₂ reacted with a hydrogen donor and 4-aminoantipyrine in a reaction catalyzed by peroxidase to form a chromophore. The lactate concentration was determined with a Beckman DXC-800(B) at an absorbance of 560 nm. The proportionality between the absorbance and concentration of lactic acid in a standard preparation was used to extrapolate the lactate concentration.

### The In Vitro Engineered Tbx18-Pacemaker Tissue Sheet

The fibroblasts and VMs were mixed in a ratio of 1:10 (fibroblast/VMs, total cell number: 1×10⁶) with an extracellular matrix of 10% Matrigel (354230, Corning®) in the cultured-insert (80209, 0.22 mm², ibidi GmbH, Grsfelfing, Germany) for gelation at 37° C. The engineered tissue was transduced with adenoviral human Tbx18 or control vectors 24 hours after gelation. The constant beating was usually observed 4-5 days after Tbx18 adenovirus transduction. Therefore, phenotypes were determined using immunofluorescence staining, beating rate (either video recording or microelectrode array), and real-time quantitative PCR after virus transduction for 5-6 days. The autonomic response of Tbx18-PC tissue sheets was evaluated by treatment with epinephrine (Taiwan Biotech Co., Ltd, Taoyuan, Taiwan).

Gene Interference by siRNAs

The Tbx18-PC cocultures were treated with 200 nM siRNAs (Dharmacon™, GE Healthcare, Lafayette, CO, USA or GenePharma, Shanghai, China) or negative controls (nontarget siRNAs, D-001810-10-05, Dharmacon™ or negative controls, GenePharma) for 24 hours using DharmaFECT transfection reagent (Dharmacon™) in serum-free and antibiotic-free medium. Cells were incubated at 37° C. in 5% C02 for 96 hours, and the beating rate was recorded by microelectrode array (MEA) or video. Then, total RNA was extracted for real-time PCR analysis. The selected genes for silencing were aldolase c (L-090123-02-0005, Dharmacon™), p38-MAPK (p38-rat-516, GenePharma), E2F1 (E2f1-rat-478, GenePharma) and Rb (Rb-rat-208, GenePharma).

### Aldolase c Overexpression

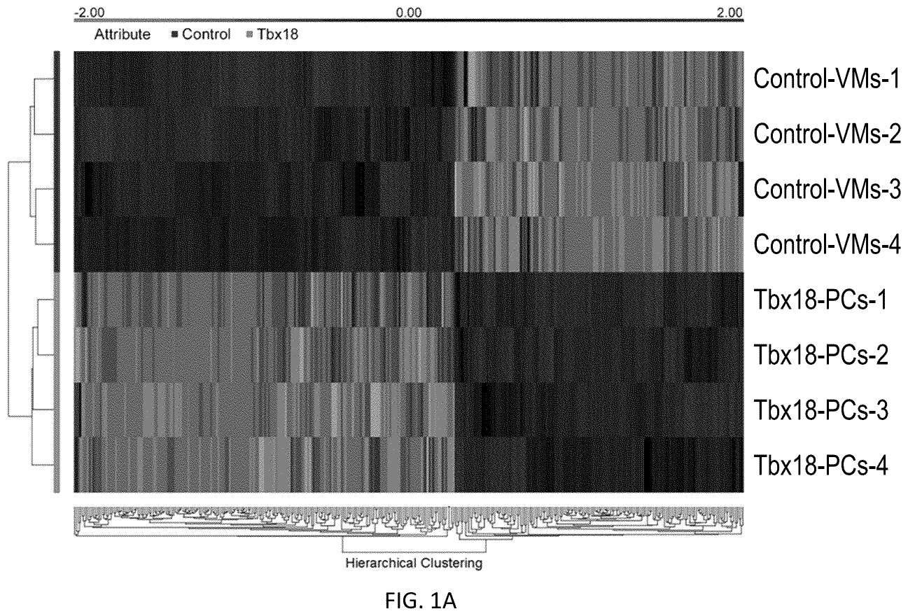

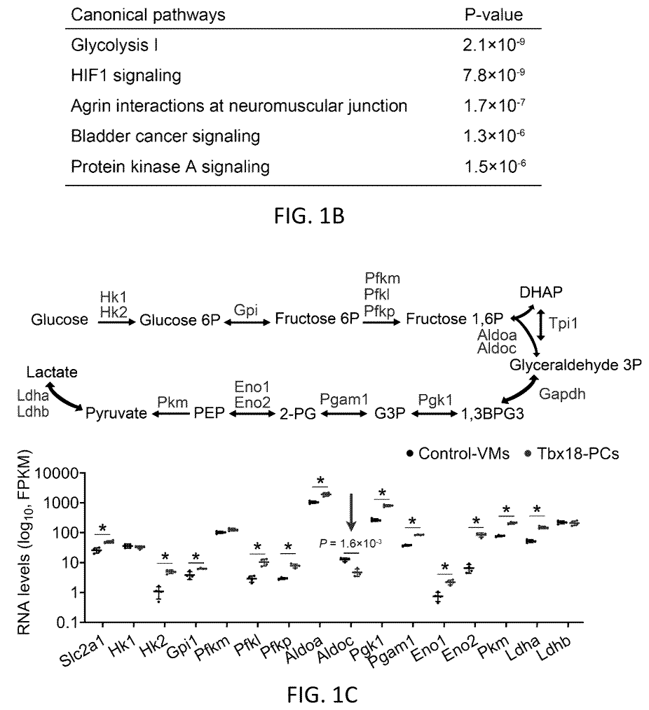

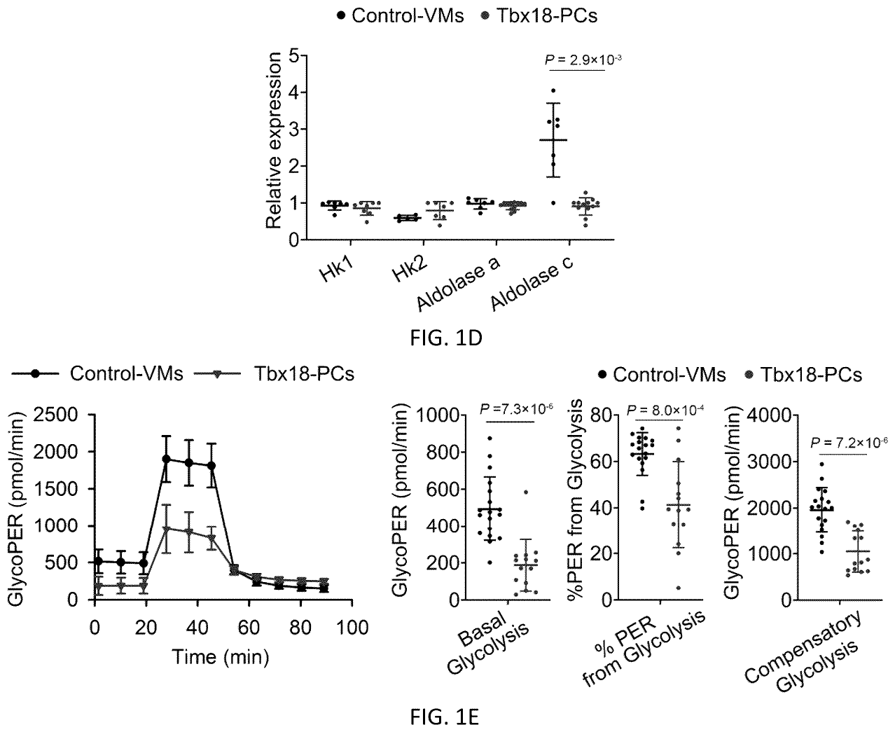

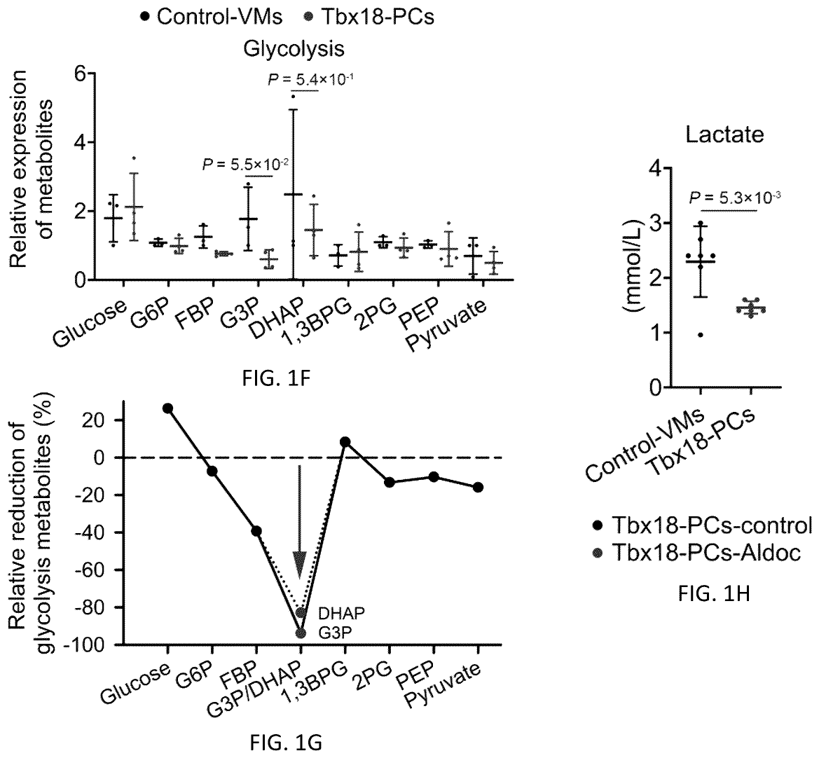

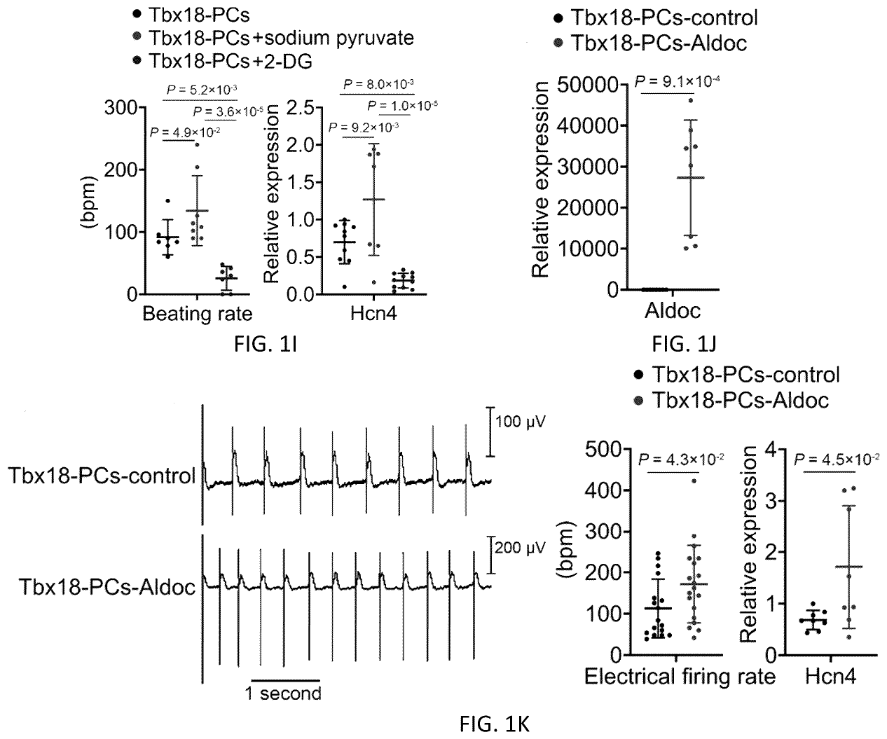

Tbx18-PCs or human IPS-CMs were transduced with human Aldoc adenovirus (CMV promotor, 068583A, Applied Biological Materials, Richmond, BC, Canada), and CMV-null adenovirus was used as a control (000047A, Applied Biological Materials) for 24 hours. Aldoc overexpression was confirmed by Aldoc transcripts (FIG. 1J). The protein, gene transcripts, and beating rates were analyzed 3 days after transduction. The viral titer used in cell transduction of aldolase c overexpression is MOI of 10. The transduced cells were not subjected to enrichment by the antibiotic selection.

### Human Induced Pluripotent Stem Cell-Derived Cardiomyocytes

Differentiation of human IPS-CMs (from Institute of Biomedical Sciences, Academia Sinica, Taiwan), dissociation, storage, and plating were performed according to previous protocols (Tsai et al.; Chiu et al.). Briefly, 6-8 μM CHIR99021 (S2924, Selleckchem, Texas, USA) was added on day 0 and day 1 in cardiac differentiation medium consisting of RPMI 1640 (11875093, Thermo Fisher Scientific) and B-27 minus insulin supplement (A1895601, Thermo Fisher Scientific). On day 2, the medium was changed to cardiac differentiation medium. On day 3, the cells were treated with 5 μM IWR-1 (10161, Sigma-Aldrich). After day 7, the medium was changed back to cardiac differentiation medium (RPMI 1640 with B-27 supplement, 17504044, Thermo Fisher Scientific), and the medium was changed every other day until dissociation. The iPSC-CMs were subjected to enrichment procedures using RPMI 1640 medium without glucose (11879020, Thermo Fisher Scientific) but with B-27 supplement for four days. Human iPS-CMs were dissociated by TrypLE Express (12605010, Thermo Fisher Science) and 1 mg/mL type IV collagenase (17104019, Thermo Fisher Science) for 10 min at 37° C., then cryopreserved in 10% DMSO and 90% FBS (16000044, Thermo Fisher Science) and stored in liquid nitrogen. For experiments described herein, human iPS-CMs were thawed and plated at low density on dishes or MEA plates in a cardiac medium consisting of RPMI 1640 and B-27 supplements. The electrical firing was recorded by MEA. Adenoviral Aldoc transduction was performed as described previously on aldolase c overexpression.

### Whole-Transcriptome Analysis for PCs

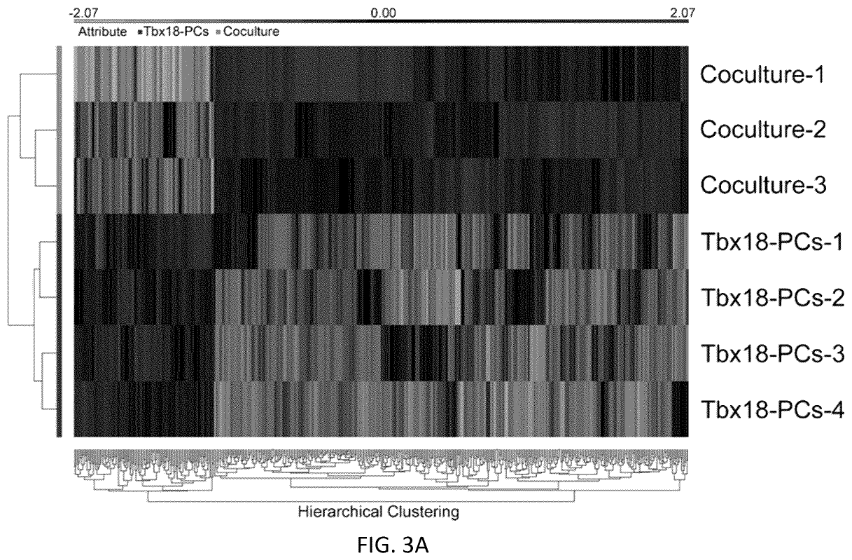

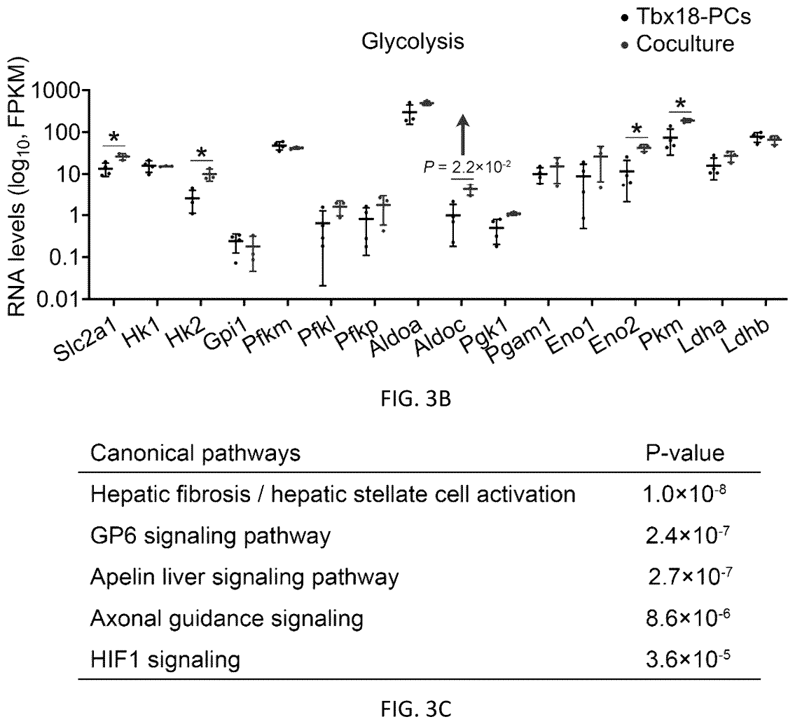

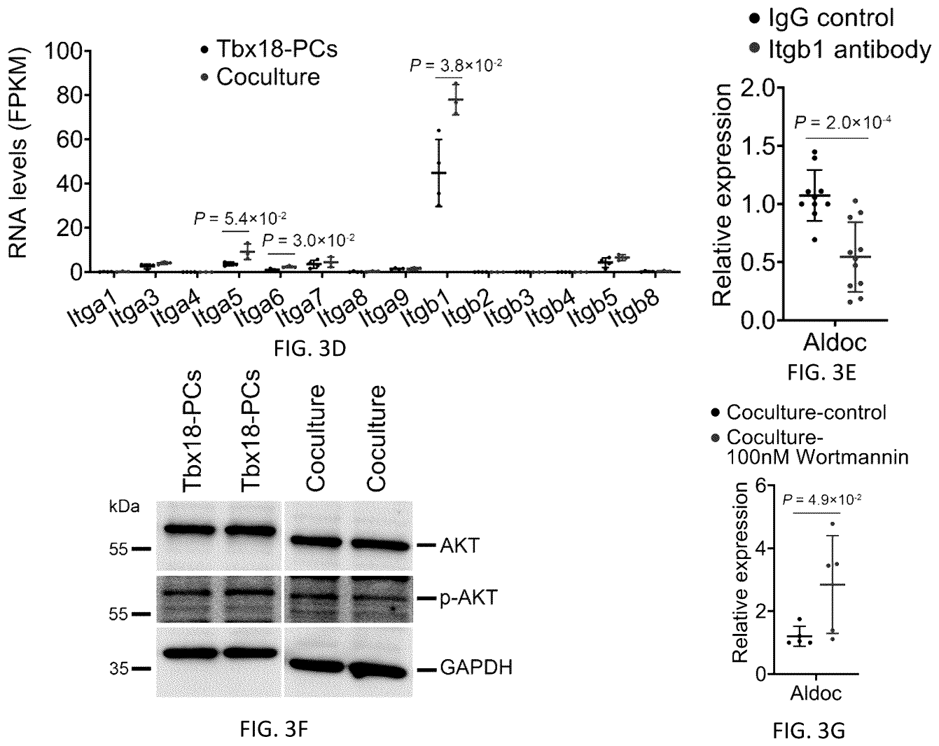

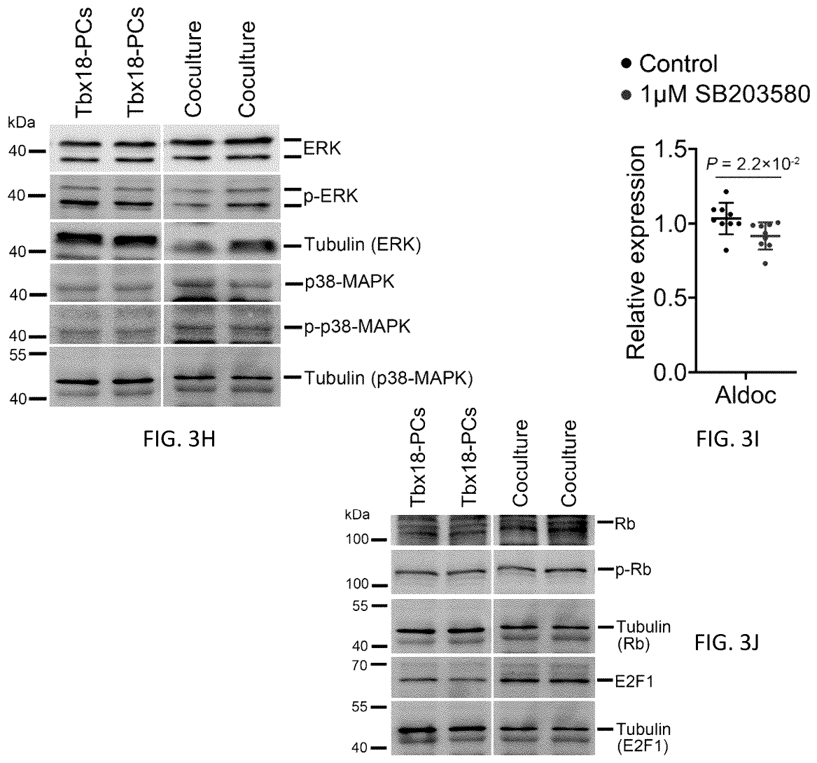

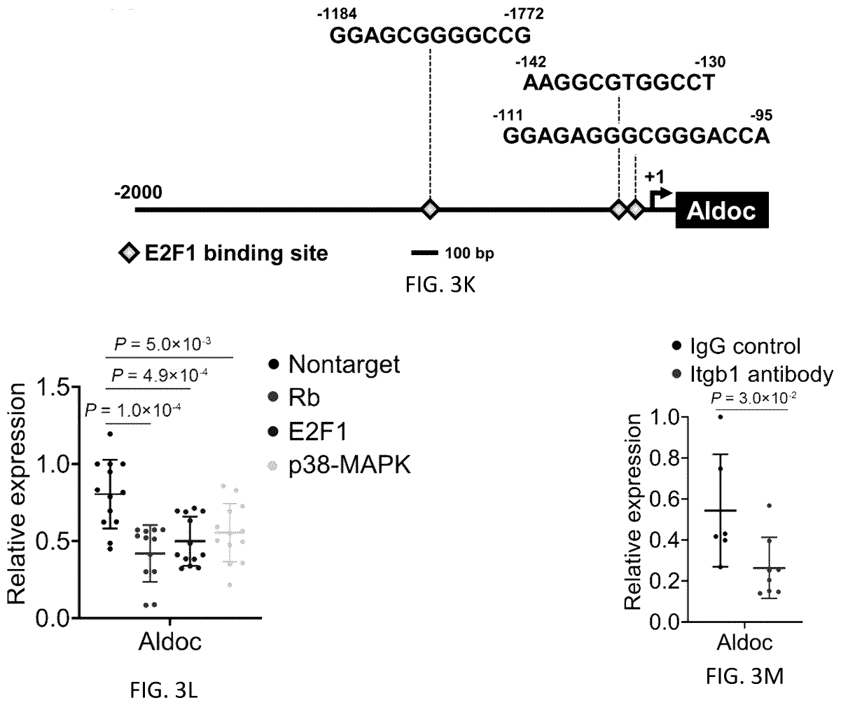

The Tbx18-induced PCs and control-VMs (FIG. 1), and sorted Tbx18-PCs from single cultures, and cocultures (FIG. 3) were used for RNA sequencing 3-4 days after Tbx18 adenoviral transduction. RNA libraries were constructed by the TruSeq RNA Library Preparation Kits (Illumina) in accordance with the manufacturer's recommendations and previous literature (Yang et al., J Mol Cell Cardiol, 2012). Briefly, 3 μg of total RNA was first purified and fragmented by poly-T oligo-attached magnetic beads. The poly-A (+) RNA was reverse-transcribed to first double-stranded cDNA using random hexamers, converted to blunt-end DNA by end repair, and then adenylated (singly) at the 3′ ends. The cDNA samples were tailed and ligated by adding barcoded adapters. Individual cDNA libraries were enriched and purified. Five to six barcoded libraries were pooled in equimolar amounts (10 nmol/L) and diluted to 4 pmol/L, ensuring that clusters were formed in a single flow cell lane. Finally, single-end sequencing was performed with a NextSeq 500 (Illumina) sequencer. After removing the adapter sequence, data was demultiplexed and libraries were converted to FASTQ formations.

The alignment of the reading sequence to the rat genome was imported into HISAT2 (graph-based alignment of next generation sequencing reads to a population of genomes, (daehwankimlab.github.io/hisat2/)) reading sequence. Each transcriptome was normalized to the length of the individual transcriptome, and the total mapped read counts in each sample, and was expressed as RNA levels. The sequence data were mapped into different isoforms of individual genes and pooled together for subsequent comparative analysis. Imported gene symbols, sequences per million mapped reads, and fragments per kilobase per million (FPKM) values were imported into MultiExperiment Viewer (MeV v4.7.4) to compare mRNA expression values. The primary function of MultiExperiment Viewer includes computation of significant levels/false discovery rates, heat-map preparation, organizing tree analyses, and hierarchical clustering.

Differentially expressed genes were applied to perform pathway and gene ontology enrichment analysis. Pathway information was extracted from NCBI BioSystems (www.ncbi.nlm.nih.gov/biosystems). The hypergeometric test was used to perform pathway enrichment analysis. Gene ontology analyses were performed using the DAVID Bioinformatics Resources 6.8 database (david.ncifcrf.gov/). The pathways were also generated using the Ingenuity Target Explorer (IPA, Qiagen, targetexplorer.ingenuity.com/).

### Seahorse Metabolic Assay

Metabolic assays, including the Seahorse XF Cell Mito Stress Test Kit (103015-100, Agilent Technologies, CA, USA) and Seahorse XF Glycolysis Stress Test Kit (103020-100, Agilent Technologies), were performed with Seahorse XFe24 Analyser (Agilent Technologies) following the manufacturer's protocol (Goodson et al., Particle and Fibre Toxicology, 2019). Tbx18-induced PCs, control-VMs, coculture of PCs or control-VMs with fibroblasts (8×10⁴ cells/well) were cultured in a 24-well microplate (precoated with fibronectin, 100850-001, Agilent Technologies). The sensor cartridge was preincubated (a day before measurement) overnight in a calibration buffer at 37° C. without CO².

Before the measures, cells were transferred into a 675 μl XF base medium (103335-100, Agilent Technologies) containing 1 mM sodium pyruvate (11360-070, Thermo Fisher Scientific), 2 mM L-glutamine (A2916801, Thermo Fisher Scientific), 20 mM glucose (G-7021, Sigma-Aldrich) and 2% serum for 30 minutes. The drugs for Seahorse XF Cell Mito Stress Test Kit included 1 μM oligomycin, 0.25 μM FCCP and 0.5 μM rotenone/antimycin A. The drugs for the Seahorse XF glycolysis stress test kit included 10 mM glucose, 1 μM oligomycin, and 50 mM 2-deoxy-D-glucose (2-DG). All the drugs were diluted with an XF base medium containing the above ingredients.

### Metabolomics Analysis

Sample preparation and analysis were performed as previously described (Chen et al., Physiol Endocrinol Metab, 2011). Tbx18-PCs, control-VMs, and cocultures of Tbx18-PCs and fibroblasts in a 6-well plate were collected. After removing the culture medium and washing, 1 ml of 80% methanol (3016-68, Macron Fine Chemicals™, Radnor Township, USA) was added to the wells on ice. Samples were collected after freezing at −80° C. for at least 15 minutes and then centrifuged (speed: 12,000 rpm) for 30 minutes at 4° C. The supernatant was transferred into an Eppendorf tube and dried by nitrogen. Residues were dissolved in 200 μL water and then centrifuged (speed: 12,000 rpm) for 30 minutes at 4° C. The samples were analyzed by Waters ultra-high-performance liquid chromatography coupled with a Waters Xevo TQ-S Mass Spectrometry (Waters Corporation, MA, USA).

To maintain a constant mass accuracy, purine, hexakis, phosphazine, or purine and formate adducts were used as internal reference ions. Data were collected in the profile mode using data acquisition software (Agilent MassHunter Software, Agilent Technologies). For structural identification of target metabolites, identical chromatographic conditions that were used in the profiling experiment were used for the metabolite standards. Mass information regarding the metabolites of interest was extracted from the raw data by using the molecular feature extraction algorithm (MassHunter, Agilent Technologies). GeneSpring-MS was used to analyze and visualize the pattern of MassHunter data matrices. The compound prediction was performed using the Metabolite Database and Molecular Formula Generation software (Agilent Technologies).

### Dihydroxyacetone Phosphate (DHAP) Fluorometric Assay

Tbx18-PCs, control-VMs, and cocultures of Tbx18-PCs or control-VMs with fibroblasts were homogenized with 100 μL ice cold DHAP Assay Buffer on ice for 10 minutes according to the manufacturer's instructions (K673-100, BioVision Inc., CA, USA). The reaction mix buffer and standard curve buffer were prepared according to the manufacturer's instructions. Samples were centrifuged at 10,000 g for 5 minutes, and 50 μL of the supernatant was mixed with the reaction mix buffer. The samples were then incubated at 37° C. for 1 hour. The fluorescence (excitation/emission=535/587 nm) was measured and used to estimate the concentration of DHAP.

### Cell Sorting of Cardiomyocytes by Flow Cytometry

Flow cytometry analysis was performed on a Becton Dickinson FACSAria Cell Sorter (Becton Dickinson Biosciences, CA, USA) with FACSComp and Cellquest software (Becton Dickinson Biosciences). Suspensions of trypsinized Tbx18-PCs or cocultures were incubated in 100 μL PBS in the dark at 4° C. for 30 min, with 1 μL of conjugated APC mouse anti-rat CD90 (561409, Becton Dickinson Biosciences), which is a surface marker of fibroblasts, but not expressed on cardiomyocytes (Pinto et al., Circ Res, 2016). After incubation, the samples were washed three times with PBS. Flow cytometry experiments were evaluated for all trypsinized cells. After a light scatter gate, cells with CD90 expression were defined by CD90 expression. Cells without CD90 expression were collected further for whole transcriptome analysis.

### Microelectrode Array (MEA)

MEA was used to record electrical firing in Tbx18-PCs, Tbx18-PC tissue sheets, or human IPS-CMs. The cells or tissue sheets were seeded on fibronectin-coated MEA CytoView MEA 24-White plate at 37° C. and 5% C02 (M384-tMEA-24W, Axion BioSystems, Atlanta, USA). The electrical firing rates were recorded by Maestro Edge (Axion BioSystems). The continuous recording of electrical signals for 5 minutes was used for the analysis (sampling frequency: 12.5 kHz, low pass and high pass filter: 2 kHz and 0.1 Hz) by AxIS Navigator software Axion BioSystems. In human IPS-CMs, the cells were further treated with epinephrine (150 ng/mL, Taiwan Biotech Co., Ltd) after recording baseline electrical firing rate. Similarly, continuous recording of electrical signals for 5 minutes after the treatment was used for the analysis.

### Quantitative Real-Time Polymerase Chain Reaction

Total RNA was extracted from cultured VMs using the RNeasy® Mini Kit (74106, Qiagen, Venlo, Netherlands). Complementary DNAs (cDNAs) were synthesized using a SuperScript® μl First-Strand Synthesis System (18080051, Thermo Fisher Science). Quantitative real-time PCR (qRT-PCR) was performed using Applied Biosystems™ StepOnePlus Real-Time PCR system with TaqMan probe assays (4331182, Invitrogen, Waltham, Massachusetts, USA) and PrimeTime qPCR Probe Assays (Integrated DNA Technologies, Inc., Coralville, Iowa, USA). The internal control was Gapdh. The conditions of thermocycle used in amplification were denaturation at 95° C. for 15 seconds and annealing and extension step at 60° C. for 60 seconds by 40 cycles. The relative values of gene transcripts were analyzed by ΔΔCt method. All the probes used are listed below in in Table 1.

TABLE 1
Probes used in quantitative real-time polymerase chain reaction

| ID | Gene Symbol | Gene Name |
|---|---|---|
| Rn00572232 | Rat Hcn4 | Hyperpolarization |
| Rn01750705 | Rat Cx45 | Connexin 45, gap junction |
| | | protein |
| Rn00562436 | Rat Hk1 | Hexokinase 1 |
| Rn00562457 | Rat Hk2 | Hexokinase 2 |
| Rn00820577 | Rat aldolase a | Fructose-bisphosphate a |
| Rn01471850 | Rat aldolase c | Fructose-bisphosphate c |
| Rn01775763 | Rat Gapdh | Glyceraldehyde-3-phosphate |
| | | dehydrogenase |
| Hs02758991_g1 | Human GAPDH | Glyceraldehyde-3-phosphate |
| | | dehydrogenase |
| Hs00902799_g1 | Human aldolase c | Fructose-bisphosphate c |
| Rn.PT.58.8261169 | Rat E2F1 | E2F Transcription Factor 1 |
| Rn.PT.58.6092134 | Rat p38-MAPK | Mitogen-activated protein |
| | | kinase 14 (Mapk14) |
| Rn.PT.58.8690996 | Rat Rb1 | Retinoblastoma protein |

### Western Blot

Cells or tissue sheets were homogenized in RIPA lysis buffer (20-188, Merck Millipore) containing protease and phosphatase inhibitors (78442, Thermo Fisher Scientific), and the supernatants were collected from the homogenates. BCA protein assays (23225, Thermo Fisher Scientific) were employed to determine the protein concentration. The proteins (30 μg/lane) were separated on a 10% SDS-PAGE gel and then transferred onto polyvinylidene difluoride membranes (PVDF, IPVH00010, Merck Millipore). Hatching with the adequate primary antibody in 5% milk/TBST was performed overnight at 4° C. All the antibodies used are listed below in Table 2. HRP-conjugated anti-mouse antibodies (1:10000, AP124P, Merck Millipore) and anti-rabbit antibodies (1:10000, 211-032-171, Jackson ImmunoResearch, Pennsylvania, USA) were used as the secondary antibodies. The bands were visualized using enhanced chemiluminescence substrates (WBKLS0500, Merck Millipore). Protein expression was analyzed using AlphaEaseFC 4.0 (Alpha Innotech, San Leandro, CA, USA).

TABLE 2
List of antibodies for western blot

Primary antibodies
Mouse anti-Aldolase c (Aldoc), ab190368, Abcam, Cambridge, UK, dilution 1:1000
Rabbit anti-p38-MAPK, 9212S, Cell Signaling Technology, Danvers,
Massachusetts, USA, dilution 1:1000
Rabbit anti-phospho-p38-MAPK (Thr180/Tyr182), 9211S, Cell Signaling
Technology, dilution 1:1000
Rabbit anti-p44/42 MAPK (Erk1/2), 9102S, Cell Signaling Technology, dilution
1:1000
Rabbit anti-phospho-p44/42 MAPK (Erk1/2, Thr202/Tyr204), 9101S, Cell Signaling
Technology, dilution 1:1000
Rabbit anti-Akt, 9272S, Cell Signaling Technology, dilution 1:1000
Rabbit anti-phospho-Akt (Ser473), 9271S, Cell Signaling Technology, dilution
1:1000
Rabbit anti-Rb, M00039-1, Boster biological technology, Pleasanton, California,
USA, dilution 1:1000
Rabbit anti-phospho-Rb (S807), P00039-1, Boster biological technology, dilution
1:1000
Rabbit anti-E2F1, ab179445, Abcam, dilution 1:1000
Mouse anti-β3 Tubulin, sc-51670, Santa Cruz Biotechnology, California, USA
dilution 1:500
Mouse anti-GAPDH, MA5-15738, Thermo Fisher Scientific, dilution 1:1000

### Immunofluorescence Staining and Reconstruction

Tbx18-PCs, tissue-sheets, or human IPS-CMs were fixed with 4% paraformaldehyde and then permeabilized with 1% Triton X-100 (X198-07, J. T. Baker, Radnor, Pennsylvania, USA) for 20 minutes at room temperature. Three percent of bovine serum albumin (BSA, Bioshop Canada Inc.) was used for blocking and was stained with primary and secondary antibodies. All the antibodies used are listed below in Table 3. Images were captured with LSM700 (Carl Zeiss AG) or FV10i (Olympus, Tokyo, Japan). The three-dimensional reconstruction of Tbx18-PC tissue sheets was made by adding all the z stacks to look at the structure of engineered tissue. The confocal images were taken by a solid-state laser at different channels: DAPI (Excitation: 405 nm, Emission: 454 nm), FITC (Excitation: 490 nm, Emission: 520 nm), CY3 (Excitation: 555 nm, Emission: 605 nm), CY5 (Excitation: 639 nm, Emission: 670 nm). The controls were employed to validate antibody specificity (isotype antibodies) and distinguish genuine target staining from the background (secondary antibody only controls).

TABLE 3
Antibodies used for immunofluorescence staining and reconstruction

Primary antibodies
Rabbit-anti-HCN4, APC-052, Alomone, dilution 1:300
Mouse-anti-α-sarcomeric actinin (ACTN2), A7811, Sigma-Aldrich, dilution 1:400
Chicken-anti-vimentin, ab24525, Abcam, dilution 1:400
Mouse-anti-Aldolase c (Aldoc), ab190368, Abcam, dilution 1:100
Mouse-anti-Aldolase c (Aldoc), sc-271593, Santa Cruz Biotechnology, dilution 1:100
Mouse-anti- Connexin 45 (Cx45), ab78408, Abcam, dilution 1:50
Secondary antibodies
Goat anti-Rabbit IgG (H + L)-Alexa Fluor 488, A-11008, Invitrogen, dilution 1:400
Goat anti-Mouse IgG (H + L)- Alexa Fluor 568, A-11004, Invitrogen, dilution 1:400
Goat anti-Chicken IgY (H + L)-Alexa Fluor 488, ab150169, Abcam, dilution 1:400
Goat anti-Mouse IgG (H + L) -Alexa Fluor 647, A-21245, Invitrogen, dilution 1:400
Goat anti-Chicken IgY (H + L)-Alexa Fluor 647, ab150171, Abcam, dilution 1:400
Donkey anti Goat IgG (H + L)-Alexa Fluor 680, A-21084, Invitrogen, dilution 1:400
Direct labeling
Rhodamine Phalloidin (F-actin), R415, Invitrogen, dilution 1:20
DAPI (4′,6-Diamidino-2-phenylindole dihydrochloride), D8417, Sigma-Aldrich, dilution
1:1000

### Analyses of Immunofluorescence Intensity

ImageJ software was used to evaluate the intensity of Aldoc expression in mouse tissue and human IPS-CMs. Aldoc expression was quantified in IPS-CMs that were positive for HCN4 and compared to levels in randomly selected cells in the same field without HCN4. Cardiomyocytes with well-defined cell borders were selected to measure the intensity of Aldoc. The nucleus was defined as positive staining for DAPI, and the total intensity of the whole cell was determined. The mouse tissues were stained with HCN4, which labeled the SAN location. Images with SAN were chosen, and the intensity of Aldoc was compared between the scramble and Aldoc knockdown groups.

### Recordings of Ca²⁺ Current

The cocultures of Tbx18-PCs and fibroblasts treated with siRNAs were stained with Ca²⁺ indicators (2 μM Rhod-2, R1245MP, Invitrogen) and incubated at 37° C. for 30 min in the dark. Fluorescence imaging was performed with a laser scanning confocal microscope (Zeiss LSM 780, Carl Zeiss). The cells were repetitively scanned over 945 μs intervals for a total duration of 7.5 s. The calcium transients were recorded, and the spontaneous localized calcium releases (LCRs) were detected using the line-scan mode along a line parallel to the longitudinal axis of a single cardiomyocyte.

### In Vivo Aldoc Knockdown by AAV9 Virus and Heart Rate Recording in Mice

Male mice (C57BL/6, 26 to 32 g, 13-14 weeks, National Laboratory Animal Center, Taiwan) were mechanically ventilated at a controlled temperature (37° C.±0.5° C.) under general anesthesia (isoflurane). A mini-thoracotomy was performed in the right parasternal area to expose the junction between the superior vena cava (SVC) and right atrium (the location of the SAN), where the small opening of the pericardium was performed. The pericardial space contains fluid-filled recesses and sinuses typically to enclose the SVC and right atrial junction (postcaval recess and superior aortic recess of transverse sinus). AAV9 siRNA or scramble virus with a reporter of green fluorescent protein (1.44×10⁹ gene copies; AAV9 scramble siRNA [iAAV01509] and Aldoc siRNA pooled virus [117530940219], Applied Biological Materials, Canada) was added to the pericardial recess through the opening. The mice were observed for 15 minutes after virus delivery, and then the wound was closed. The survival rate was 100%. All animals were included except one scramble animal. One animal from the scramble group was excluded because arrhythmia was observed before the experiment.

Two weeks later, the three-lead surface ECG (Biopac, MP36, CA, USA) was recorded on mice by inserting needle electrodes subcutaneously into the limbs after anesthesia. The heart rate was recorded for 2 minutes first to compare the knockdown and control groups. Then, we performed a peritoneal injection of epinephrine (2.5 μg, Taiwan Biotech) and recorded the heart rate for 5 minutes after the injection. After the recording, the mouse hearts were removed for tissue analysis. ECG data were analyzed by Biopac Student Lab 4.1 software (Biopac).

### The Treatment of Ltgb1 Inhibitory Antibody in Mice

The 16-week-old C57BL/6 mice were injected intraperitoneally with a function-blocking anti-mouse ltgb1 antibody (0.1 ml of 0.05 mg/mL, LEAF™ purified anti-mouse CD29 Armenian hamster IgG (102202, clone HMB1-1, Biolegend, San Diego, CA, USA) or control IgG (400902, clone HTK888, Biolegend) for three days. SANs were collected for the extraction of RNAs and quantitative real-time PCR. All animals were included without any exclusion.

In the experiments of in vivo Aldoc knockdown by AAV9 virus and the treatment of ltgb1 inhibitory antibody in mice, the impact of sex difference was yet elucidated. Therefore, the protocol which used male animals only was designed. Future studies to exclude sex differences are needed. The sample size was estimated to achieve a power of 0.8 and α-level=0.05 using a two-sided t-test. The animal and samples were randomly allocated into experiment groups. Because the number of our lab members is too small, the researchers need to design and perform the experiment themselves. Therefore, the design of blindness cannot be performed. However, all experimental results were analyzed by two independent investigators.

### Statistical Analysis

Statistical analysis was performed with GraphPad Prism version 8.3.0 (GraphPad Software, CA, USA). Data are expressed as the mean±SD. The Shapiro-Wilks normality test was used to determine the use of Student's t-test, one-way analysis of variance or Mann Whitney test as appropriate. Repeated measures ANOVA with the LSD post hoc test was used when repeated measures were necessary. Multiple comparison correction was performed using the false discovery rate control approach and the Benjamini-Hochberg method when multiple comparisons of different variants were needed. A P-value less than 0.05 was considered statistically significant.

### Results

### Glycolysis Metabolism Regulated Rhythmicity in Tbx18-Induced Pacemaker Cardiomyocytes

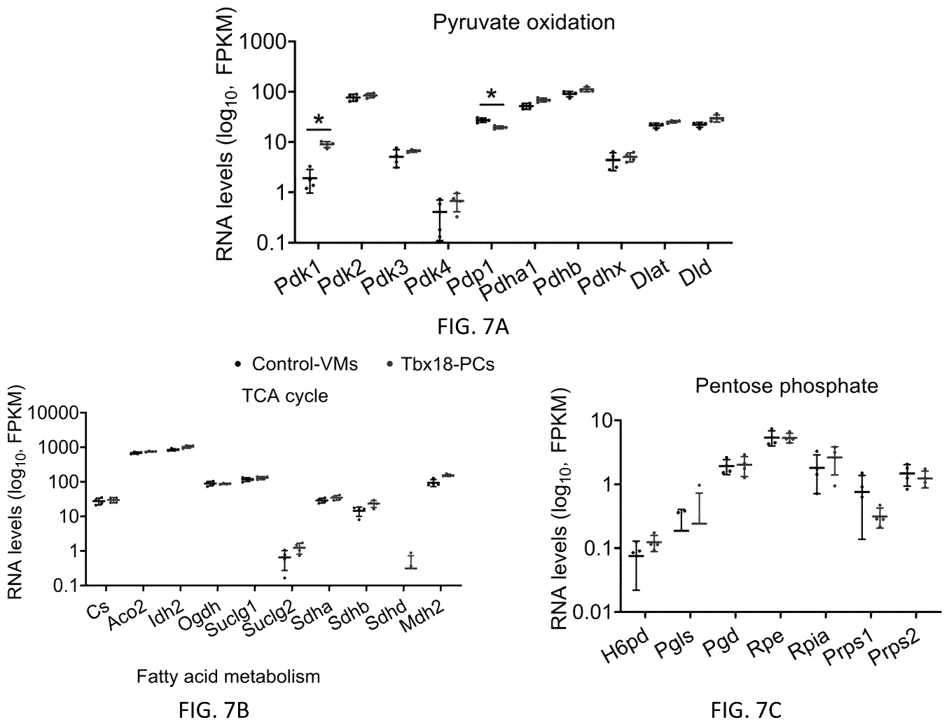

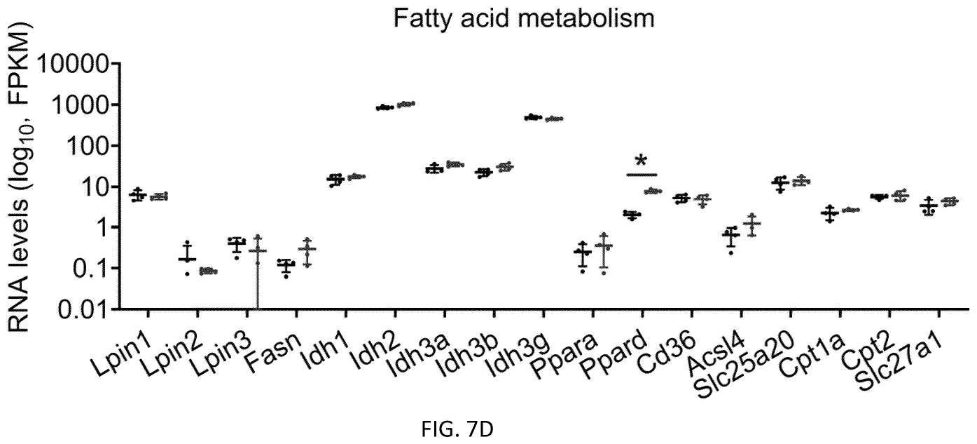

It was hypothesized that a cell-specific biological process of PCs, which is different from that of quiescent ventricular cardiomyocytes, would underlie the tailored integration of fibroblasts and PCs for functional integrity (Bressan et al., Cell Rep, 2018; Furtado et al., Development, 2016). Therefore, Tbx18-induced PCs (Tbx18-PCs) were selected as the cell model (Kapoor et al.; Hu et al., Sci Transl Med, 2014)). The differential biological processes between PCs and ventricular cardiomyocytes (VMs) were explored. Whole-transcriptome expression was compared between Tbx18-PCs and control-VMs (FIGS. 7A-7D) (Kapoor et a.; Hu et al.). The whole transcriptome analysis dataset is provided in the Sequence Read Archive (SRA) data at NCBI (PRJNA743181 and PRJNA743409).

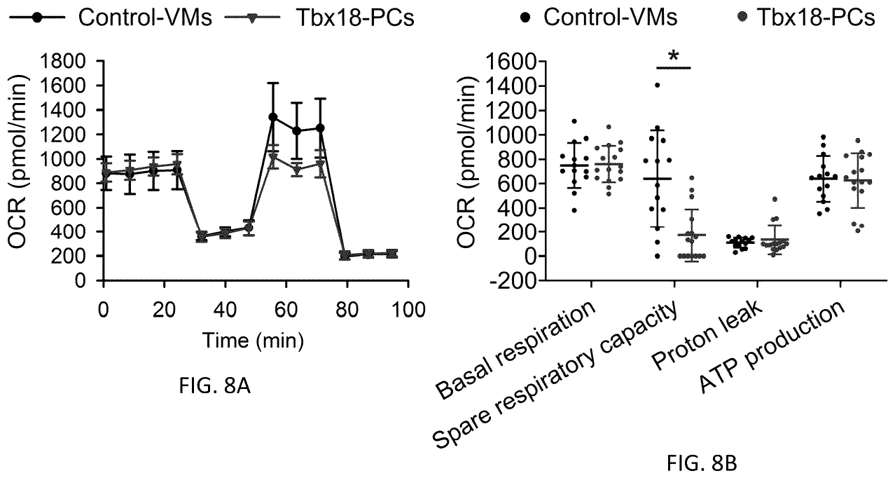

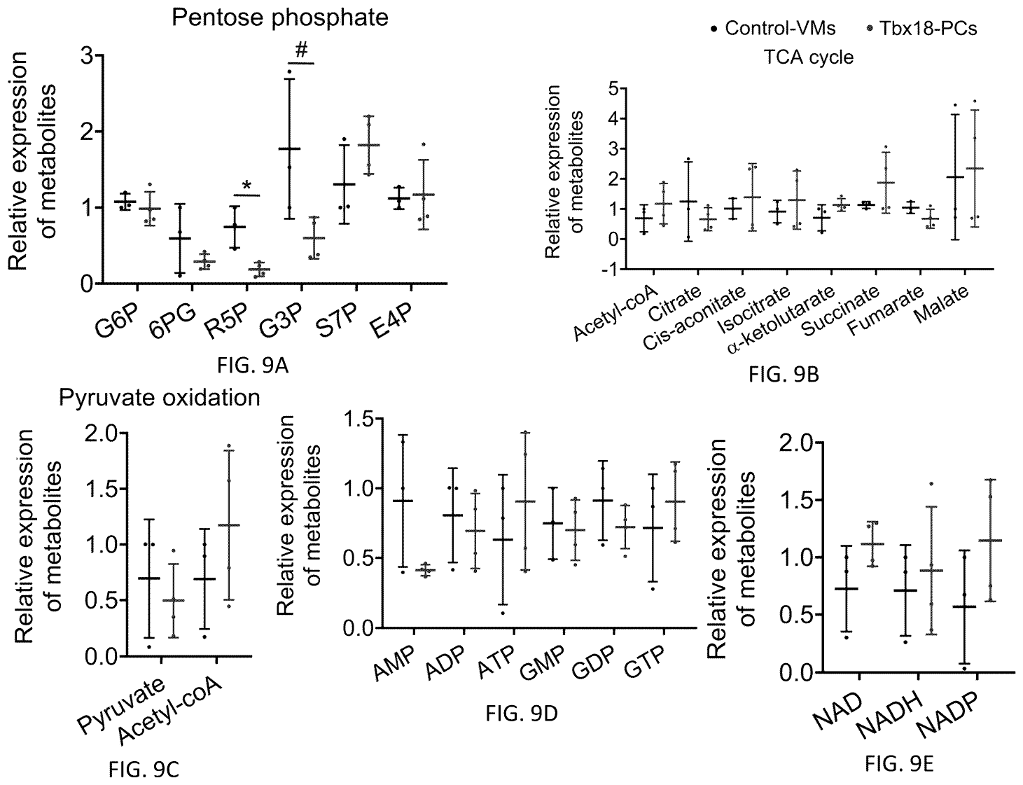

Glucose metabolism and glycolysis accounted for the top canonical pathways (FIGS. 1B and 8A-8B, Table 4) and were predominant in gene ontology analysis (Table 5; FIGS. 9A-9E). Glucose is metabolized to pyruvate via a complex enzyme network (FIG. 1C). The metabolic genes involved in glycolysis were mostly increased in Tbx18-PCs. Aldolase c was an exception, as its transcripts significantly decreased (FIG. 1C). The genes in other metabolic pathways, including the tricarboxylic acid cycle (TCA) cycle, pentose phosphate pathway, pyruvate oxidation, and fatty acid metabolism were either not different or slightly increased between Tbx18-PCs and control-VMs (FIG. 7A-7D). The differential change in Aldoc transcripts between Tbx18-PCs and control-VMs was confirmed via real-time PCR (FIG. 1D).

TABLE 4
Top canonical pathways of the whole-transcriptome analysis between
Tbx18-pacemarkers and control ventricular cardiomyocytes

| | P-value | Overlap |
|---|---|---|
| Glycolysis I | 2.1 × 10⁻⁹ | 30.8%, 8/26 |
| HIF1 signaling | 7.8 × 10⁻⁹ | 8.3%, 17/205 |
| Agrin interactions at neuromuscular | 1.7 × 10⁻⁷ | 12.8%, 10/78 |
| junction | | |
| Bladder cancer signaling | 1.3 × 10⁻⁶ | 10.3%, 10/97 |
| Protein kinase A signaling | 1.5 × 10⁻⁶ | 5.0%, 20/399 |

TABLE 5
Top biological processes from gene ontology of the
whole-transcriptome analysis between Tbx18-pacemakers
and control ventricular cardiomyocytes

| | P-value | Overlap |
|---|---|---|
| Glycolytic process | 4.5 × 10⁻⁵ | 25.6%, 10/39 |
| Cellular response to hypoxia | 6.7 × 10⁻³ | 9.5%, 13/137 |
| Negative regulation of transcription, | 8.1 × 10⁻³ | 4.9%, 26/532 |
| DNA-templated | | |
| Potassium ion transport | 1.4 × 10⁻² | 13.2%, 9/68 |
| Positive regulation of apoptotic process | 1.6 × 10⁻² | 5.6%, 19/338 |

To correlate functional changes in transcriptome expression, glycolysis and mitochondrial function in Tbx18-induced PCs were further analysed using Seahorse functional assays. Glycolysis activity, including basal and compensatory glycolysis, and proton efflux rate in Tbx18-PCs were lower than in control-VMs (FIG. 1E). Mitochondrial function, including basal respiration, proton leak and ATP production did not differ between the two groups (FIGS. 8A-8B). The downregulation of glycolysis was correlated with the reduced levels of Aldoc and suggested that the increased expression of metabolic genes other than Aldoc was likely compensatory. To confirm this idea, metabolomics analysis was performed via liquid chromatography-mass spectrometry (LC/MS) to comprehensively delineate the metabolite levels of the aforementioned pathways in Tbx18-PCs and control-VMs.

The levels of TCA cycle metabolites, energy molecules (e.g., ATP and NADH), pyruvate conversion metabolites, and pentose phosphate pathway metabolites were mostly not statistically different between Tbx18-PCs and control-VMs (FIGS. 9A-9E). Only within the glycolysis process, almost all metabolites decreased. A maximal decrease was observed in the levels of glyceraldehyde-3-phosphate (G3P) and dihydroxyacetone phosphate (DHAP, FIG. 1F), which reached the nadir of all glycolysis metabolites (FIG. 1G). Aldoc catalyses reversible aldolase cleavage of fructose 1,6-bisphosphate (FBP) to DHAP and G3P (FIG. 1C) (Arakaki et al., Protein Sci, 2004). The critical reduction in DHAP and G3P levels was consistent with the reduction in Aldoc expression. The reduction in lactate levels (end-product of glycolysis) was further confirmed by a colorimetric assay (FIG. 1H). These results reflected low glycolysis activity in the single culture of PCs and demonstrated the key role of reduced Aldoc expression in determining glycolysis status.

It was then determined whether glycolysis activity in PCs has functional implications. Supplementation with sodium pyruvate to replenish the energy supply from glycolysis in Tbx18-PCs increased the beating rates of PCs and Hcn4 expression (distinct ion channels that initiate electrical impulses in PCs, FIG. 1I). Treatment with 2-deoxy-D-glucose (2-DG) to inhibit glycolysis decreased both beating rates and Hcn4 expression in Tbx18-PCs (FIG. 1I). Considered a critical hub in the glycolysis process, adenoviral vector-mediated overexpression of Aldoc was performed to reverse Aldoc expression in Tbx18-induced PCs (FIG. 1J). The increased expression of Aldoc improved beating rates and Hcn4 expression (FIG. 1K). These results indicated that reduced Aldoc levels and glycolysis status dysregulated PC rhythmicity.

### Fibroblasts Drive Glycolysis Adaptation Through Aldolase c in Tbx18-Induced Pacemaker Cardiomyocytes

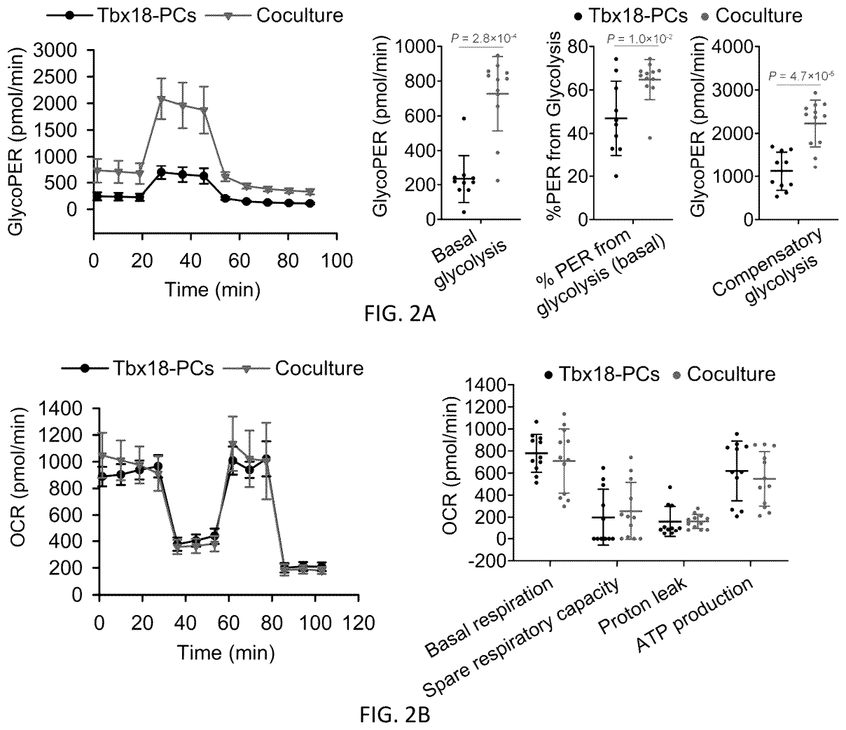

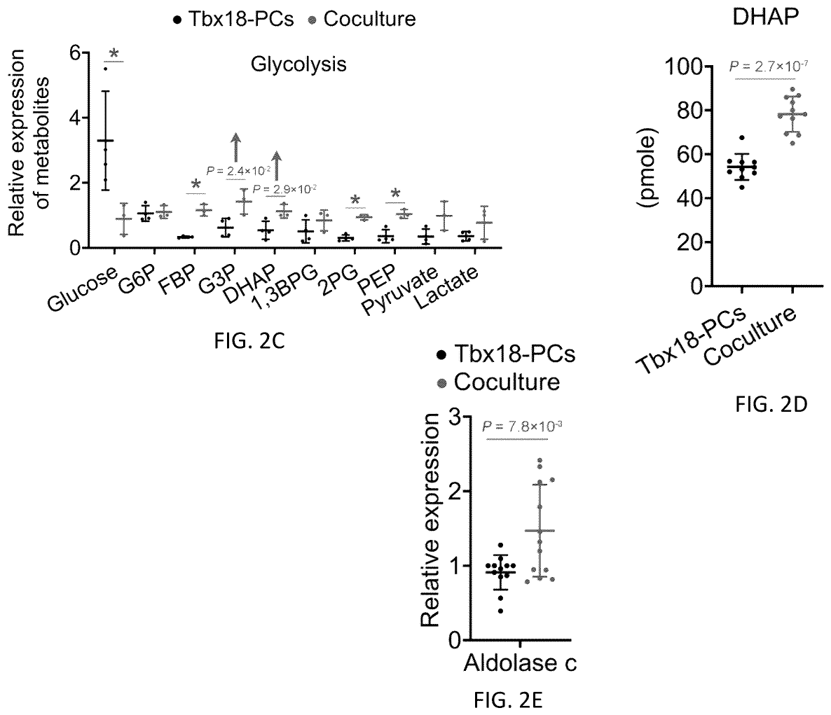

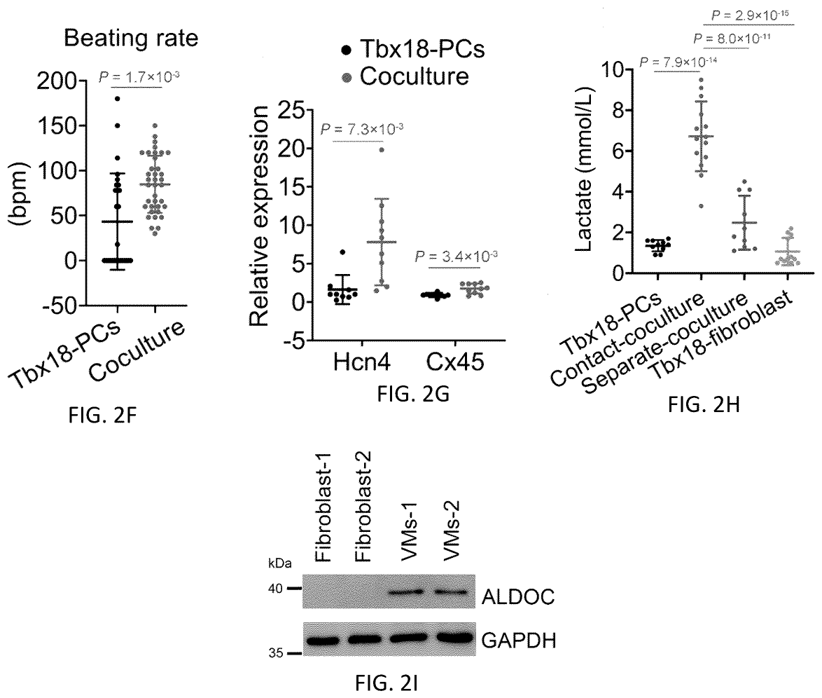

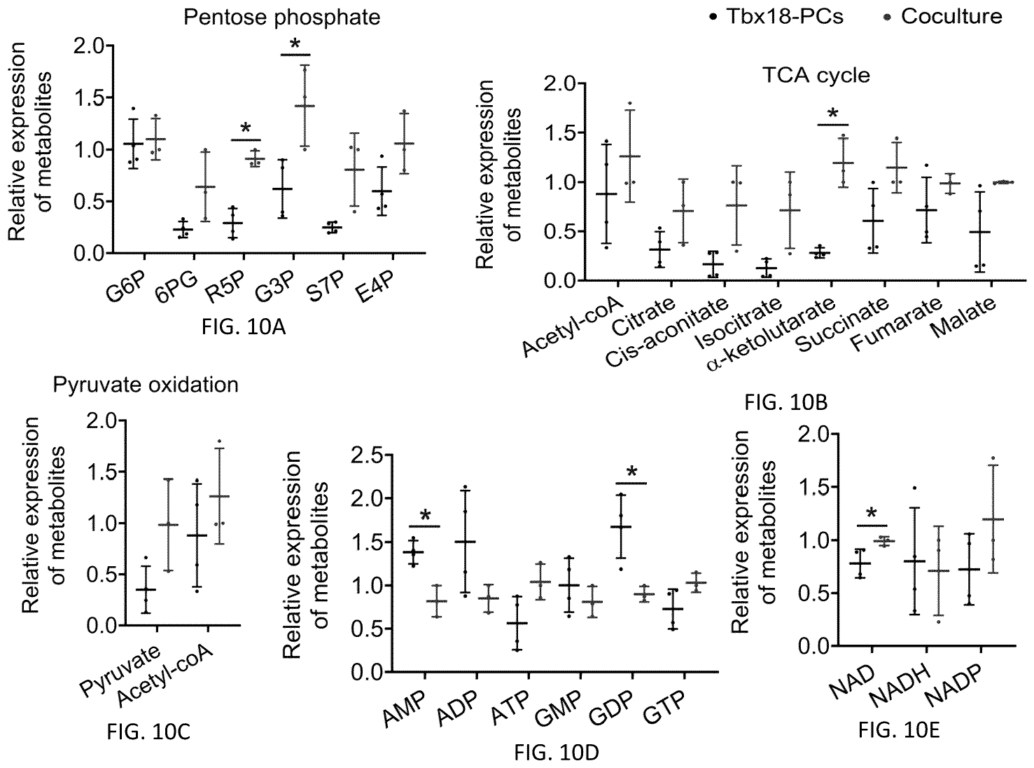

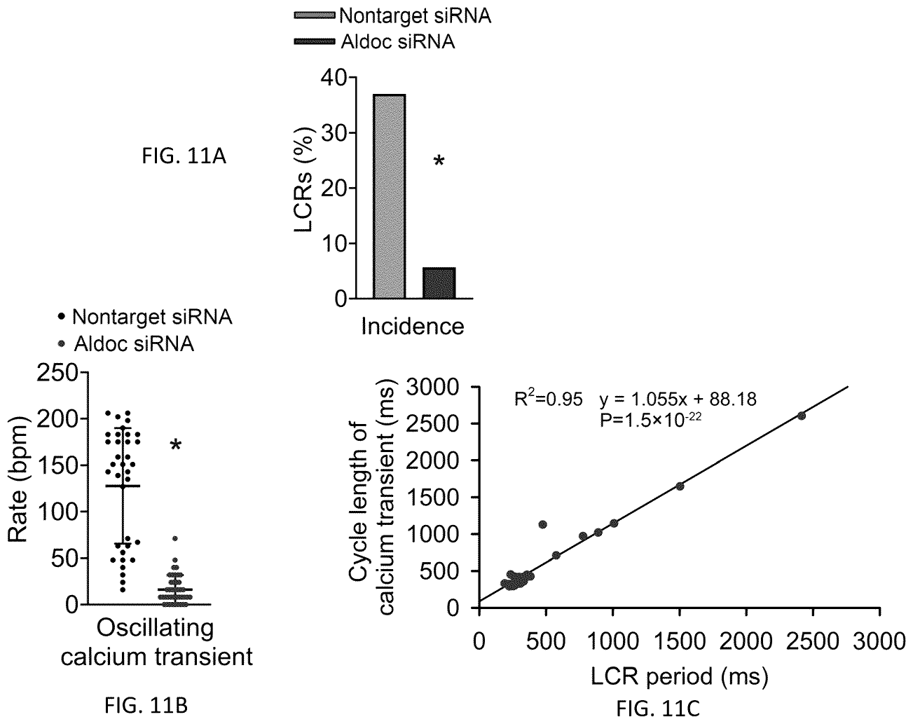

Next, it was determined whether fibroblasts regulate pacemaker rhythm through intrinsic glycolysis within Tbx18-PCs. Coculture with fibroblasts improved global glycolysis function, including basal and compensatory glycolysis, as well as the proton efflux rate, compared to Tbx18-PCs alone (FIG. 2A). Mitochondrial function (oxidative phosphorylation), including basal respiration, spare respiratory capacity, proton leak and ATP production did not differ between the two groups (FIG. 2B). Based on metabolomics analysis, compared to the drastic decrease in G3P and DHAP in the single culture of Tbx18-PCs, coculture with fibroblasts significantly increased G3P and DHAP, as well as FBP, through the reverse reaction of aldolase from DHAP (FIG. 2C) (Choi et al., Biochemistry, 2001). This led to the upregulation of downstream metabolites, such as 2PG (2-phosphoglyceric acid) and PEP (phosphoenolpyruvate). The levels of TCA cycle metabolites, energy molecules (e.g., ATP and NADH), pyruvate conversion metabolites, and pentose phosphate pathway metabolites were mostly marginally increased or not statistically different between Tbx18-PCs and cocultures (FIGS. 10A-10E). The increased levels of DHAP were validated by an enzyme-linked immunosorbent assay (ELISA, FIG. 2D). Aldoc expression in the cocultures was higher than that in the single PC cultures (FIG. 2E), thus supporting the notion that Aldoc expression underlies the increase in G3P and DHAP levels. In addition, coculture with fibroblasts was associated with better pacemaker phenotypes such as beating rate (FIG. 2F), as well as with the expression of PC-specific genes (Hcn4 and connexin 45 [Cx45], FIG. 2G). Spontaneous local Ca²⁺ release events (LCRs) are a hallmark of automaticity in PCs (Hu et al., Nat Biomed Eng, 2021; Lakatta et al., Circ Res, 2010). LCRs could be observed in Tbx18-PC cocultures. The inhibition of Aldoc by the Aldoc siRNAs decreased both LCRs and oscillating calcium transients (FIGS. 11A-11C). The LCR period, as observed in the Tbx18-PC cocultures, was linearly correlated with the cycle length of oscillating calcium transients (FIGS. 11A-11C). Overall, these findings suggest that Aldoc regulates both membrane and calcium clocks within PCs.

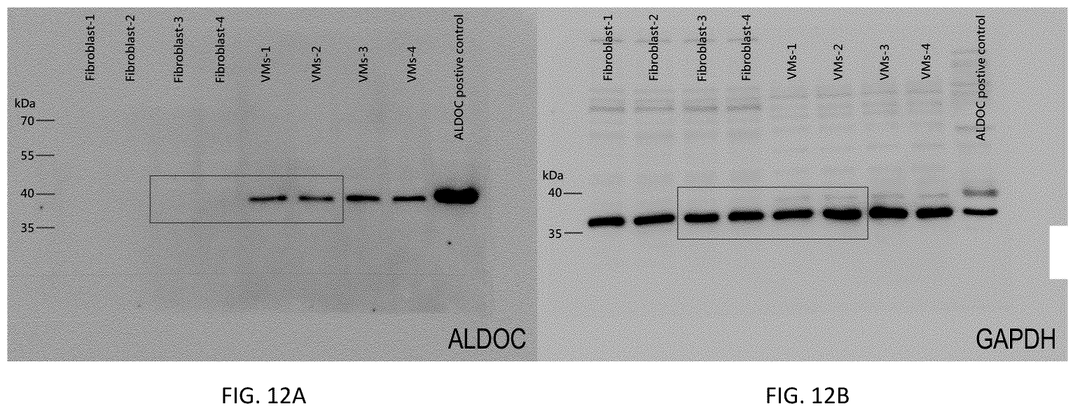

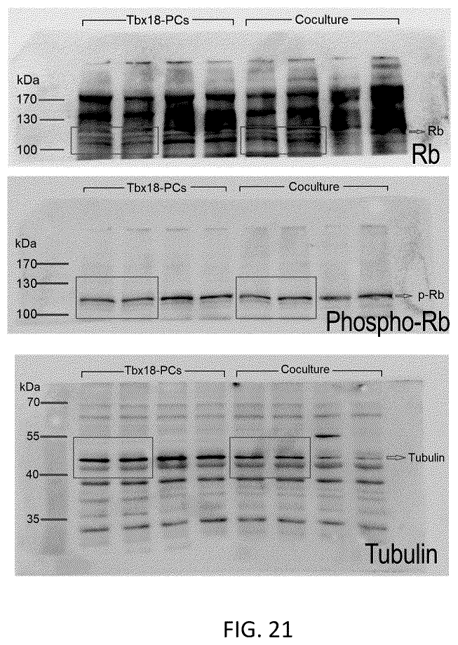

The improvement in glycolysis was related to the intrinsic regulation of PCs but not to contamination of fibroblasts. First, lactate levels increased in contact cocultures, which supported the improvement of glycolysis function after coculture with fibroblasts (FIG. 2H). However, if a separate coculture was performed (coculture of fibroblasts and PCs, but PCs and fibroblasts were separated by porous membranes), the levels of lactate did not increase. Lactate levels in the single culture of fibroblasts were also low. These findings indicated that the microenvironment contributed to the improvement in glycolytic activity through contact between fibroblasts and PCs. Moreover, no Aldoc protein expression was observed in fibroblasts (FIG. 21 and FIGS. 12A-12B). These results indicated that the improvement in glycolysis was due to intrinsic regulation of Aldoc within PCs.

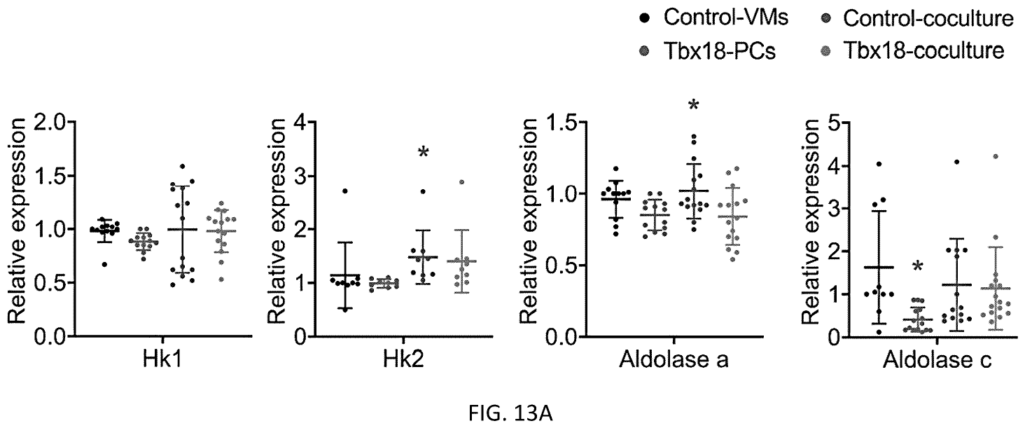

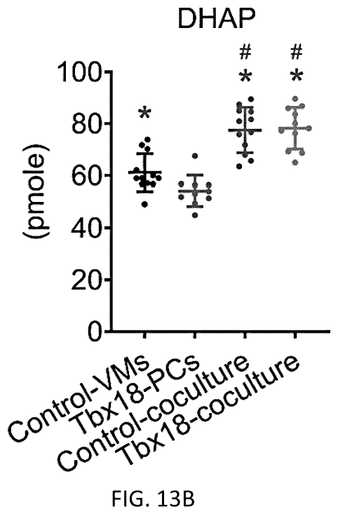

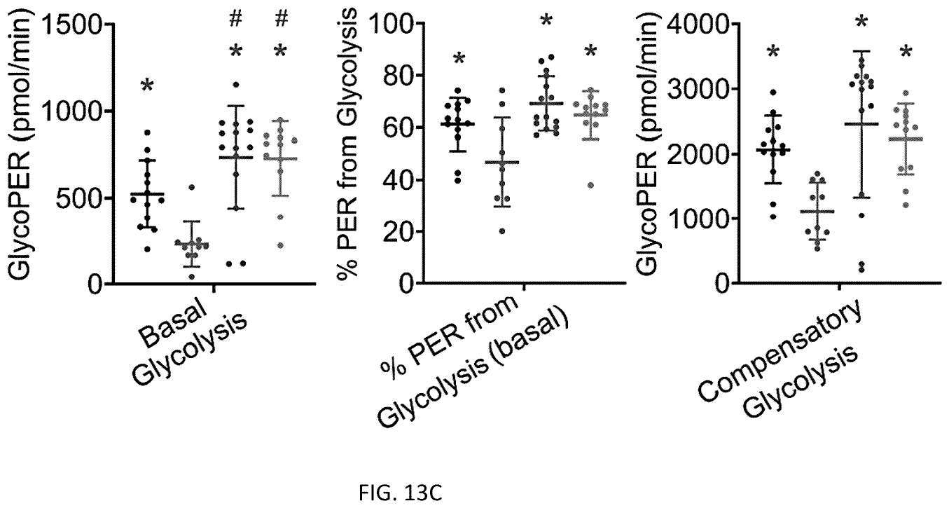

The regulatory enzymes of glycolysis in Tbx18-PC cocultures were different from those in cocultures of control-VMs and fibroblasts (FIGS. 13A-13C). In contrast to increased Aldoc expressions within Tbx18-PCs, fibroblasts increased the transcripts of aldolase a in control-VMs after coculture. Therefore, glycolysis function and DHAP levels improved in both Tbx18-PCs and control-VMs after coculture with fibroblasts (FIGS. 13A-13C).

### Fibroblasts Switched on Aldoc Expression in Pacemaker Cardiomyocytes Through Integrin-Dependent Signals

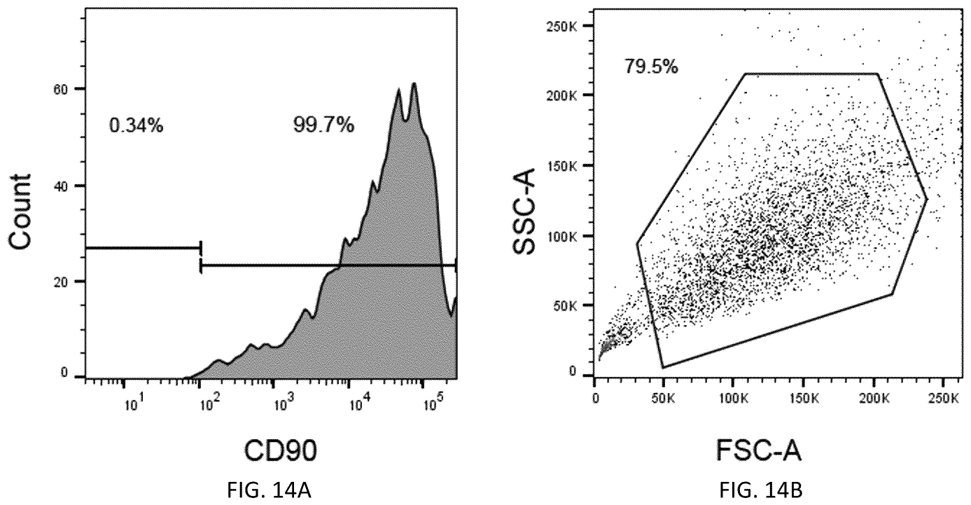

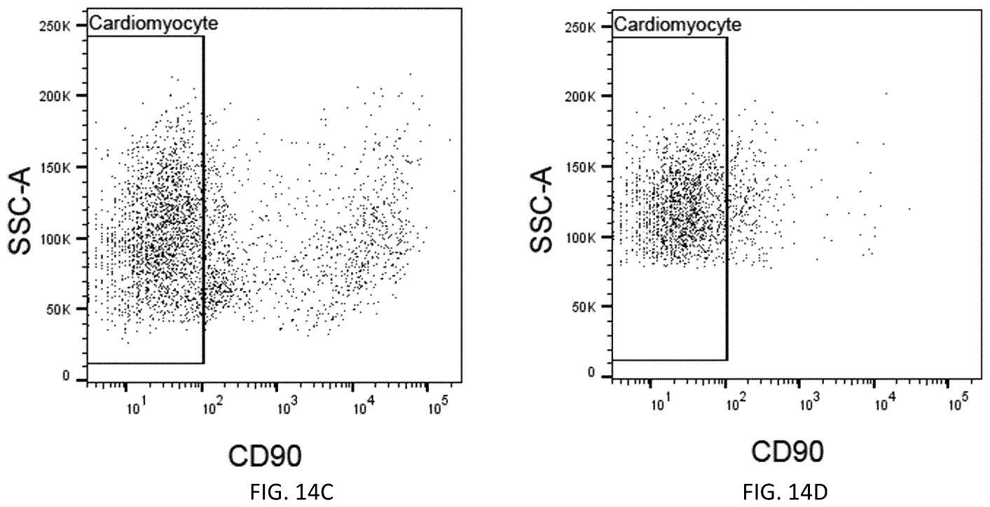

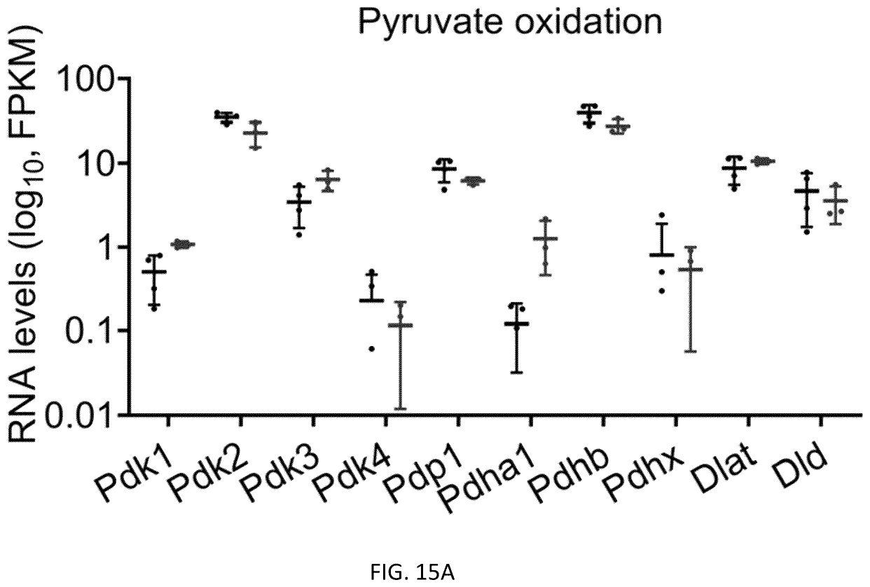

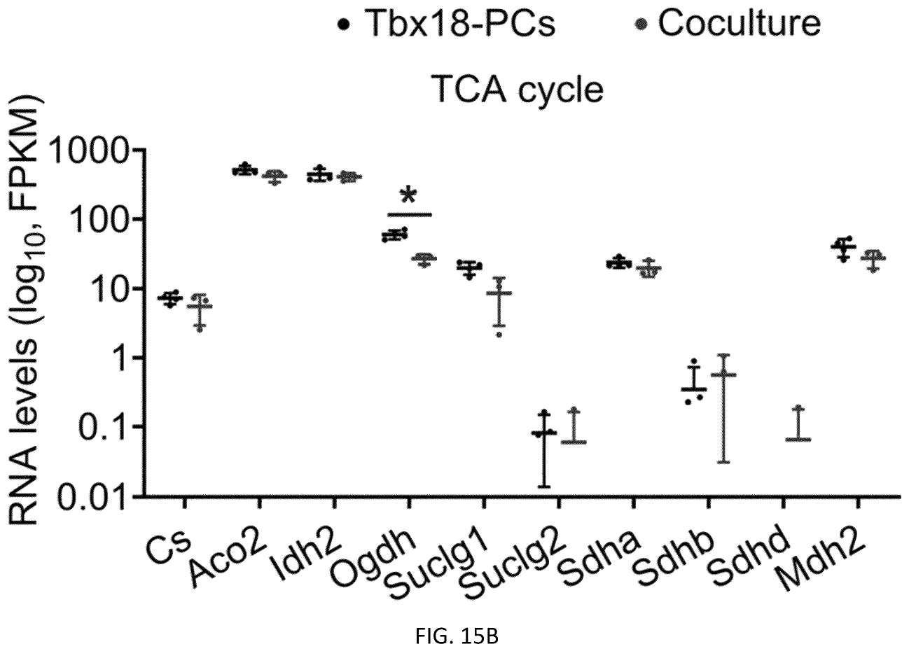

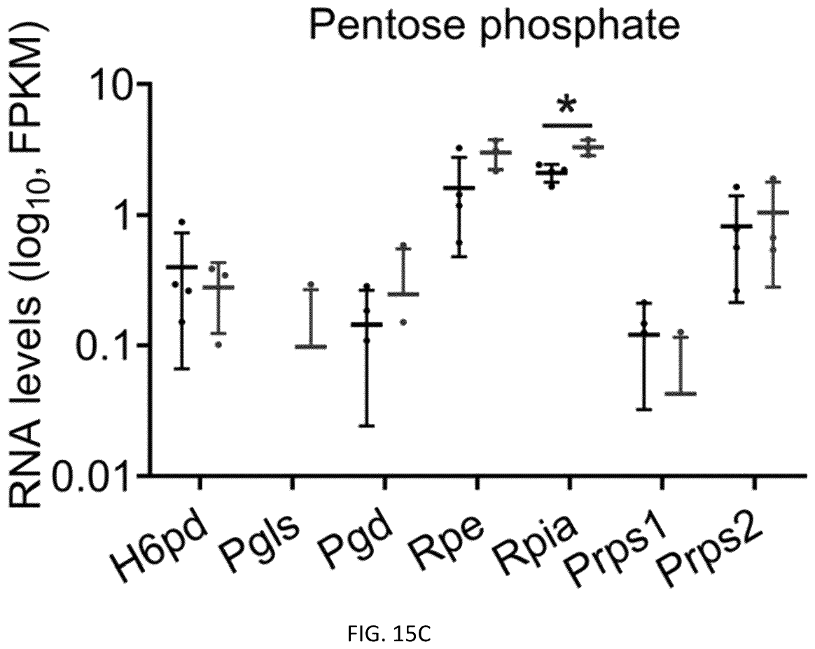

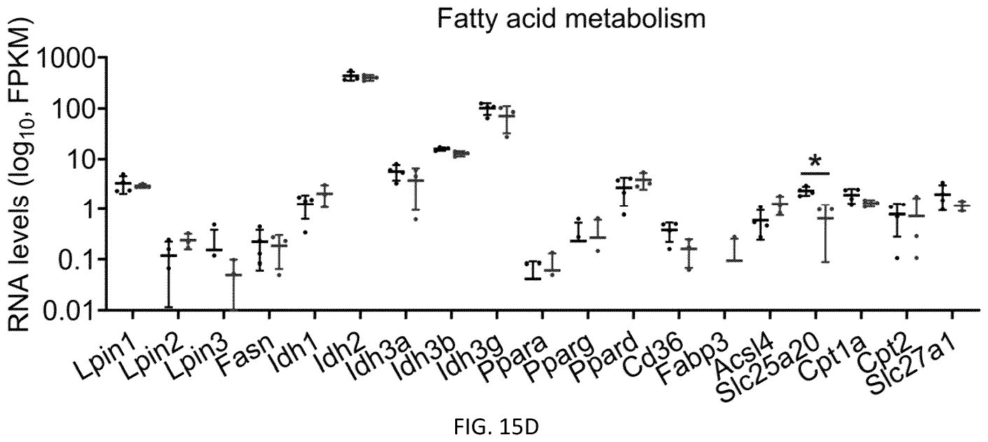

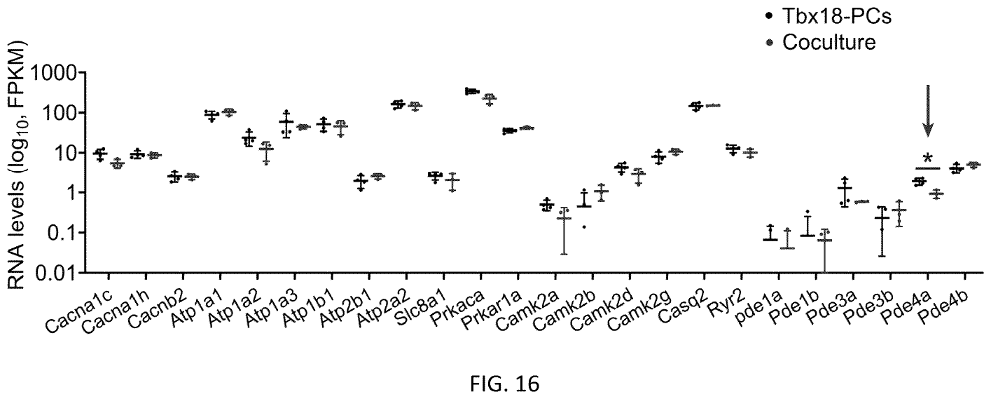

The mechanisms by which fibroblasts regulate Aldoc expression in PCs were further explored. After coculture with fibroblasts, PCs were isolated via cell sorting and the results are shown in FIGS. 14A-14D. The whole transcriptome expression in isolated PCs from PC-fibroblast cocultures was compared to that from PCs in single PC cultures. The whole transcriptome analysis dataset is provided in the Sequence Read Archive (SRA) data at NCBI (PRJNA743181 and PRJNA743409). The analysis of glycolysis-related genes revealed that the highest transcriptional changes (4.3-fold increment) were observed at Aldoc levels compared to the other glycolysis enzymes (FIG. 3B). Other metabolic genes related to pyruvate oxidation, tricarboxylic acid cycle, the pentose phosphate pathway, and fatty acid metabolism were either minimally or not statistically different (FIGS. 15A-15D). Again, Aldoc was the key enzyme critically regulated by fibroblasts. The relevant pathways related to metabolic/energetic regulation of automaticity, mainly those within the calcium clock, were analyzed (FIG. 16) (Lakatta et al., Yaniv et al., J Mol Cell Cardiol, 2011). The spontaneous electrical activity of PCs, especially calcium clock, is driven by a cAMP-mediated phosphorylation, which critically relies on the balance between the cAMP production by adenylyl cyclases and degradation by cyclic nucleotide phosphodiesterases (Pde). The coculture with fibroblasts decreased the expression of phosphodiesterase (Pde4a) and might regulate cAMP-mediated protein phosphorylation and the calcium clock in PCs (Vinogradova et al., Circ Arrhythm Electrophysiol, 2018).

Ingenuity Pathway Analysis (IPA) was performed to explore the pathways to potentially regulate Aldoc expression, including canonical pathways and gene ontology (FIG. 3C, Tables 6 and 7). Within canonical pathways, those related to extracellular matrix (ECM, e.g., collagen, and laminin), relevant surface receptors (integrins) and their downstream signals (phosphoinositide 3-kinases [PI3K]/Akt and mitogen-activated protein kinase [MAPK]) were repeatedly observed. These results were compatible with the finding that direct contact between fibroblast-PCs and the extracellular matrix is critical to drive metabolic reprogramming in PCs. Therefore, integrins, which are the predominant ECM-binding receptors in cardiomyocytes, were comprehensively analyzed (FIG. 3D) (Israeli-Rosenberg et al., Circ Res, 2014). The ltgb1 (integrin subunit 131) transcripts were the most abundant and increased significantly. Tbx18-PC cocultures were further treated with an ltgb1 inhibitory antibody, which decreased integrin activation, Aldoc expression (FIG. 3E), and the beating rate (control vs. ltgb-1 antibody: 140.4±92.0 bpm, n=23 vs. 20.6±55.7 bpm, n=16, P=1.2×10⁵). This indicates that fibroblasts activate Aldoc and glycolysis activity through integrin-dependent signals.

TABLE 6
Top canonical pathways of the whole-transcriptome analysis between isolated
Tbx18-pacemaker cardiomyocytes from single cultures and cocultures

| | | | Adhesion proteins, |
|---|---|---|---|
| | | | extracellular matrix, |
| | P-value | Overlap | and signal pathways |
| Hepatic fibrosis/hepatic | 1.0 × 10⁻⁸ | 13.2%, 21/159 | Collagen family genes |
| stellate cell activation | | | |
| GP6 signaling pathway | 2.4 × 10⁻⁷ | 15.0%, 15/100 | Collagen family genes, |
| | | | laminin, MAPK pathway, |
| | | | PI3K-AKT pathway |
| Apelin liver signaling | 2.7 × 10⁻⁷ | 33.3%, 8/24 | Collagen family genes, |
| | | | MAPK pathway |
| Axonal guidance | 8.6 × 10⁻⁶ | 7.1%, 30/420 | Integrins, PI3K-AKT and |
| signaling | | | MAPK pathways |
| HIF1 signaling | 3.6 × 10⁻⁵ | 12.1%, 12/99 | PI3K and MAPK pathways |

TABLE 7
The top biological process from gene ontology of the whole-
transcriptome analysis between isolated Tbx18-pacemaker
cardiomyocytes from single cultures and cocultures

| | P-value | Overlap |
|---|---|---|
| Response to hypoxia | 1.3 × 10⁻⁵ | 10.0%, 27/270 |
| Angiogenesis | 1.3 × 10⁻⁵ | 12.0%, 22/184 |
| Endodermal cell differentiation | 1.6 × 10⁻⁵ | 35.7%, 10/28 |
| Collagen fibril organization | 2.8 × 10⁻⁵ | 27.5%, 11/40 |
| Positive regulation of cell | 1.6 × 10⁻⁴ | 20.2%, 21/104 |
| migration | | |

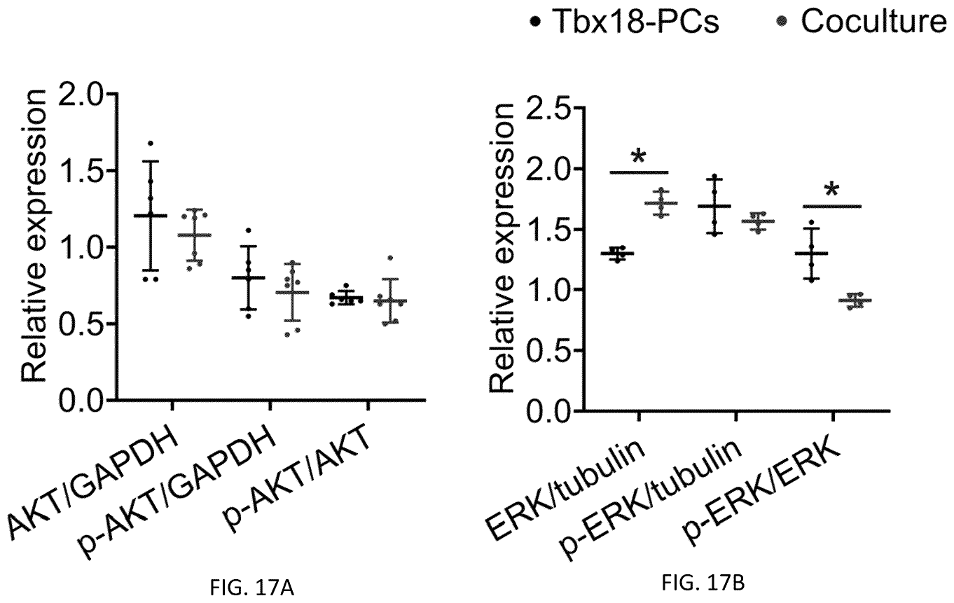

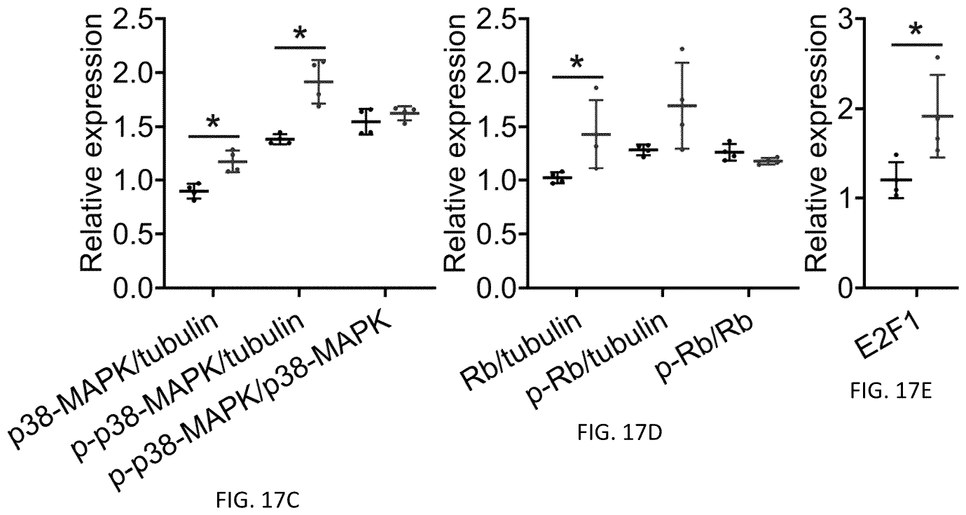

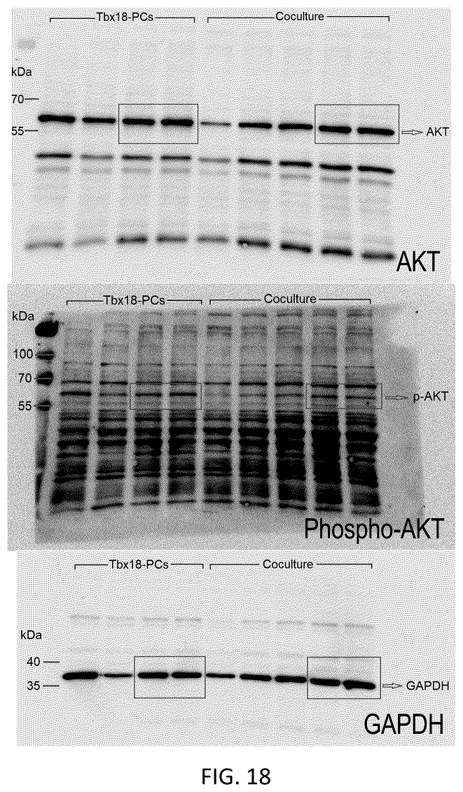

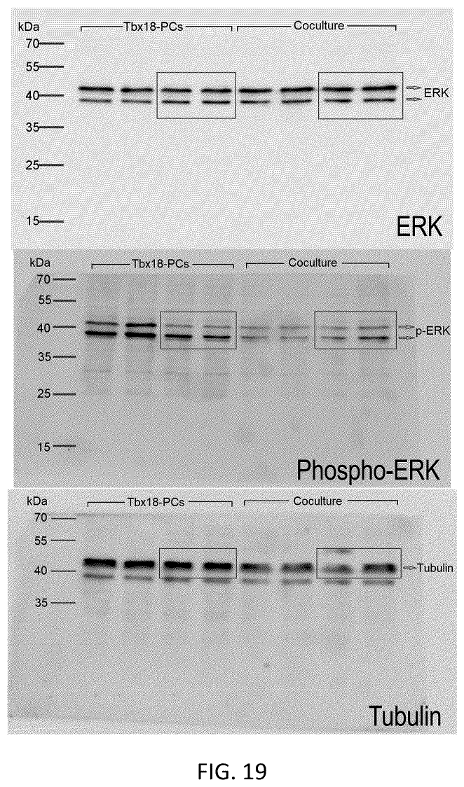

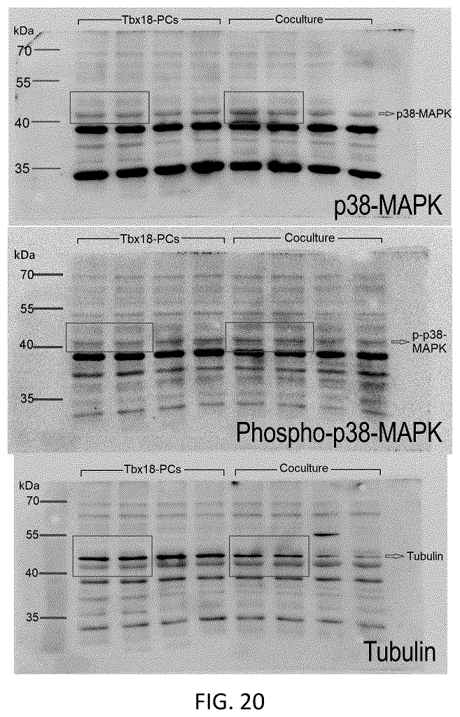

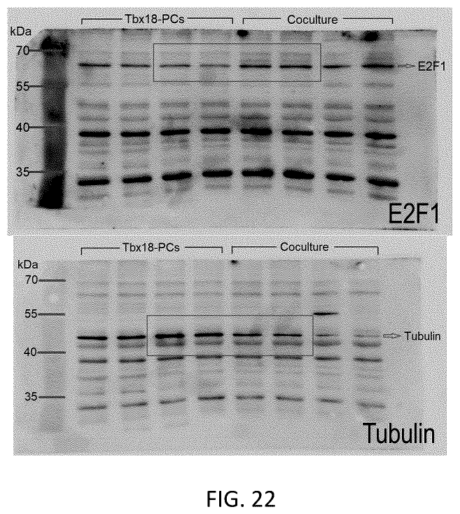

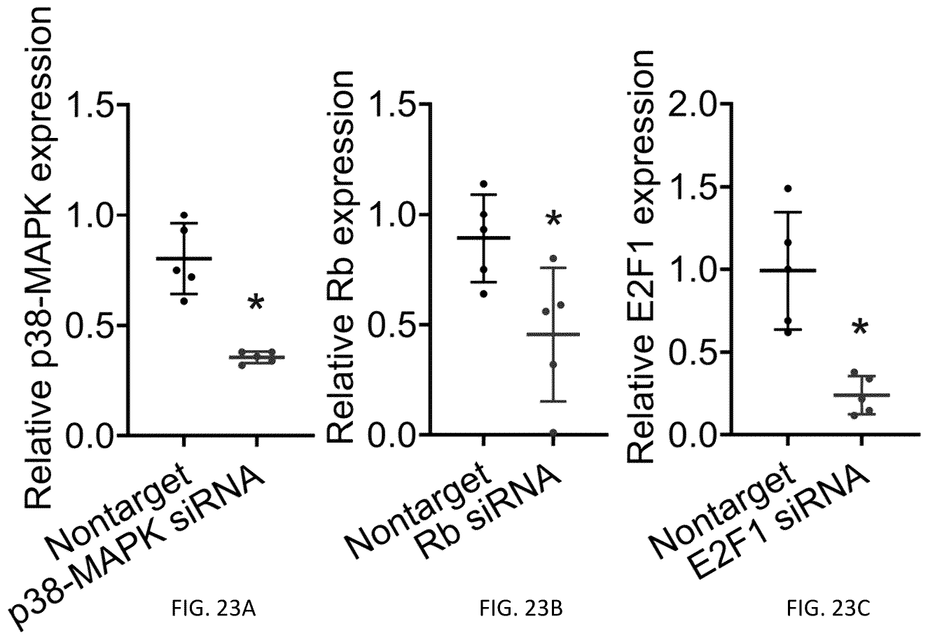

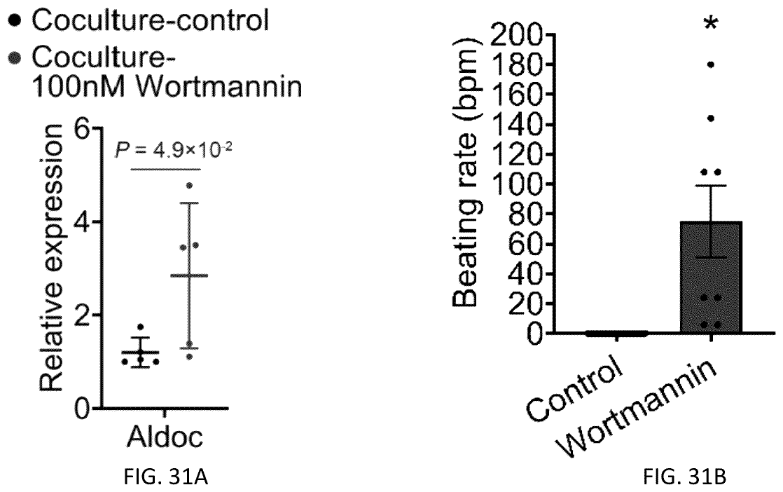

The common downstream signals of integrins within cardiomyocytes include phosphoinositide 3-kinase (PI3K)/Akt, and MAPK (p38 and ERK) (Israeli-Rosenberg et al.; Roux et al., Microbiol Mol Biol Rev, 2004). The expression of phosphorylated and total Akt did not change between cocultures and single cultures of PCs (FIGS. 3F, 17A and 18). Treatment with a PI3K inhibitor (wortmannin) in PC cocultures increased Aldoc expression (FIG. 3G). These results indicate that PI3K/Akt activity did not activate Aldoc expressions after coculture. Considering MAPK pathways, ERK activation was suppressed after coculture as phosphorylated ERK (p-ERK) was lower in cocultures than in the single culture of PCs (FIGS. 3H, 17B and 19). Instead, phosphorylated and total p38-MAPK increased after coculture (FIGS. 3H, 17C and 20). Treatment of cocultures with a p38-MAPK inhibitor (SB203580) decreased Aldoc expression, suggesting that p38-MAPK activation is a downstream signal of integrins (FIG. 31) to increase Aldoc expression. The p38-MAPK activation induces Rb-E2F1 dissociation and increases E2F1-driven transcriptional activity (Wang et al., EMBO J, 1999). Increased expression of Rb, phosphorylated Rb, and E2F1 was observed in cocultures (FIGS. 3J, 17D-17E, and 21-22). There are multiple E2F1 binding sites on the promotor region of Aldoc, suggesting that E2F1 is a transcriptional regulator of Aldoc (FIG. 3K). Inhibition of p38-MAPK, Rb, and E2F1 via siRNA decreased Aldoc expression (FIGS. 3L and 23A-23C). An in vivo experiment was further performed to clarify the link between Aldoc expression and integrin-dependent signaling. Mice that received an intraperitoneal injection of the ltgb1 inhibitory antibody (a functional blocking antibody) had lower Aldoc expression in SANs than control mice (FIG. 3M). Overall, these results suggest that fibroblasts induce Aldoc expression in PCs through β1-integrin activation and downstream p38-MAPK/E2F1 signaling.

### Engineered Tbx18-PC Tissue Sheets Recapitulated Aldoc-Driven Rhythmic Machinery

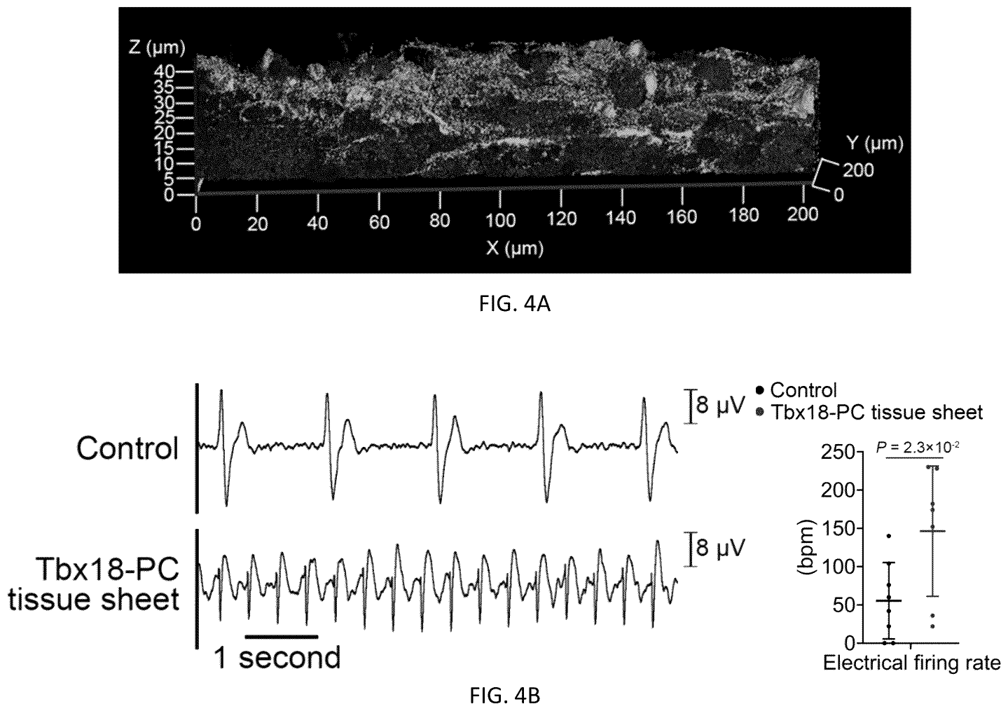

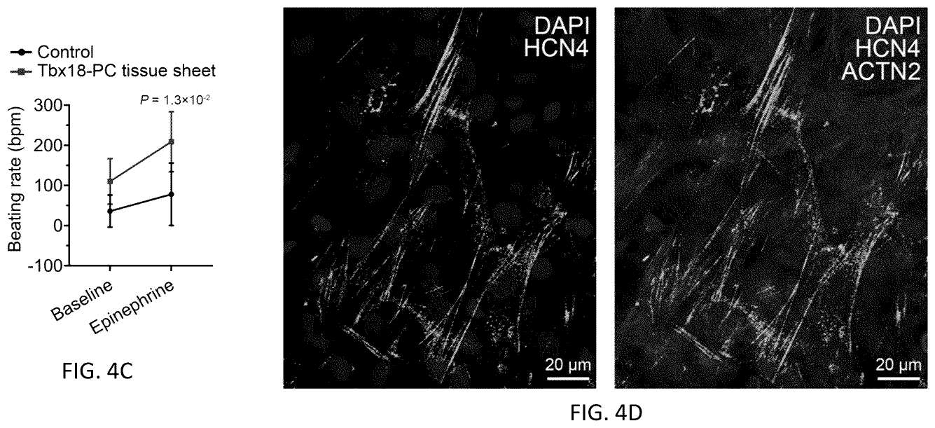

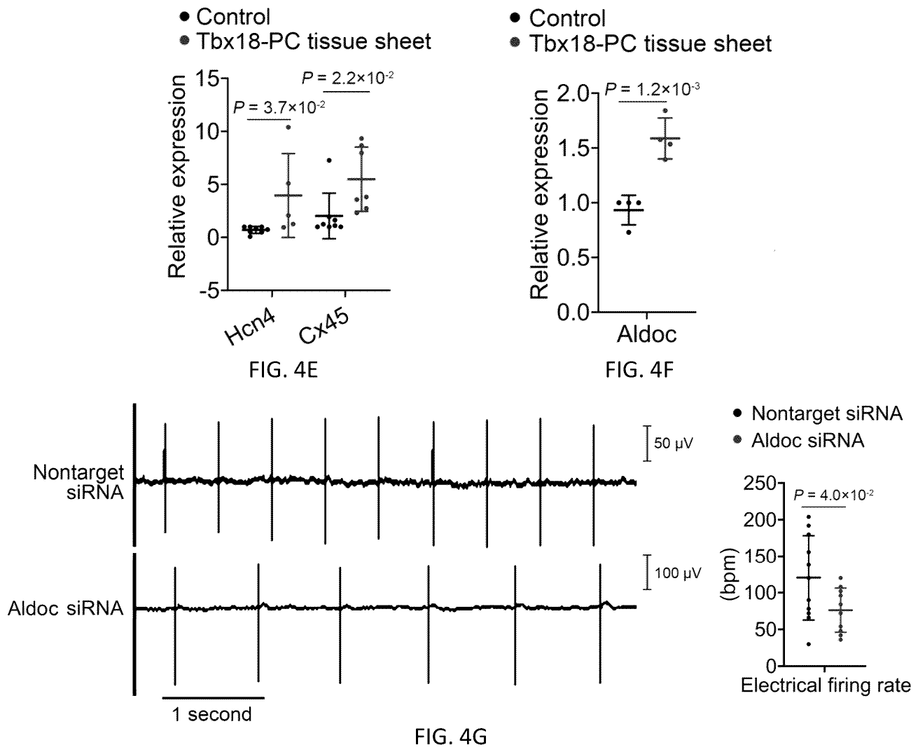

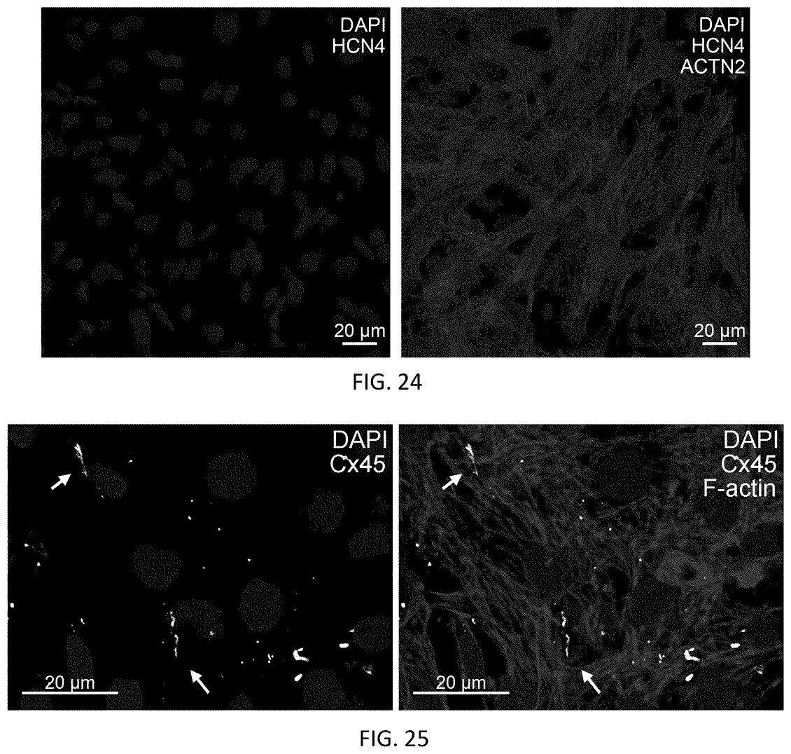

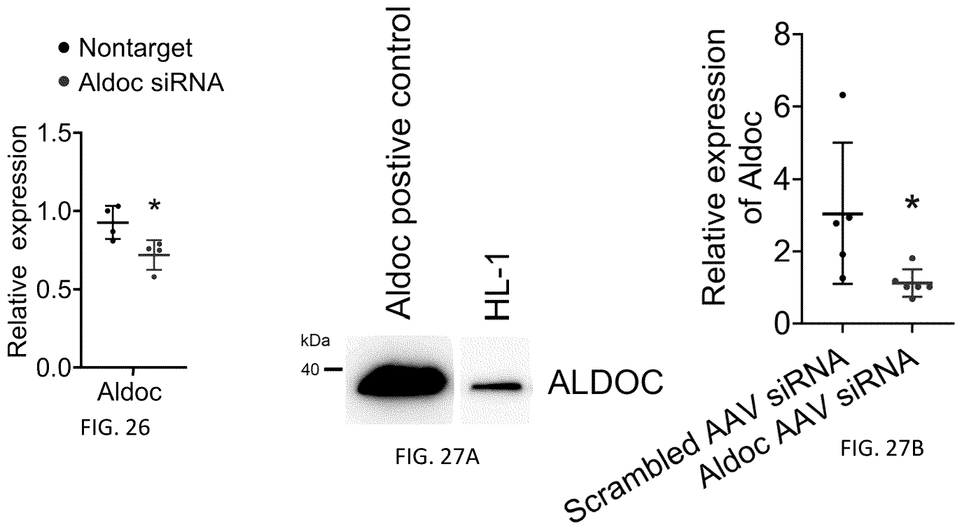

The microenvironment with fibroblasts is essential for the functional integrity of pacemaker tissue. An in vitro Tbx18-PC tissue sheet was constructed to mimic the three-dimensional microenvironment of in vivo SANs to study the regulatory role of Aldoc in pacemaker rhythmicity. The PC tissue sheet was induced by re-expression of Tbx18 in an engineered tissue, which was constructed by mixed culture of VMs and fibroblasts with Matrigel (FIG. 4A). Compared to control tissue sheets (GFP expression), re-expression of Tbx18 induced spontaneous electrical firing that was recorded with a microelectrode array (MEA, FIG. 4B). A sympathomimetic drug (epinephrine, α- and β-receptor sympathetic agonist) increased the beating rate of Tbx18-PC tissue sheets (FIG. 4C). Through immunofluorescence staining, PCs with distinct pacemaker ion channels (HCN4, FIGS. 4D and 24) and Cx45 (FIG. 25) were observed in Tbx18-PC tissue sheets but not in controls. PC tissue sheets also had higher expression of PC-specific genes (Hcn4 and Cx45) than control tissue sheets (FIG. 4E). These findings suggest that engineered Tbx18-PC tissue sheets recapitulated the phenotypes of a native SAN. Aldoc-driven rhythmic machinery within a three-dimensional microenvironment was further explored. Aldoc expression in PC tissue sheets was higher than controls (FIG. 4F). Treatment of PC tissue sheets with Aldoc siRNAs reduced Aldoc expression (FIG. 26) and spontaneous electrical activity (FIG. 4G). Engineered Tbx18-PC tissue sheets recapitulated the microenvironment of de novo SAN tissues and suggested that the Aldoc-driven glycolysis machinery regulates PC rhythmicity in a three-dimensional microenvironment.

### The Regulation of In-Vivo Pacemaker Rhythms by Aldolase c in Vertebrates

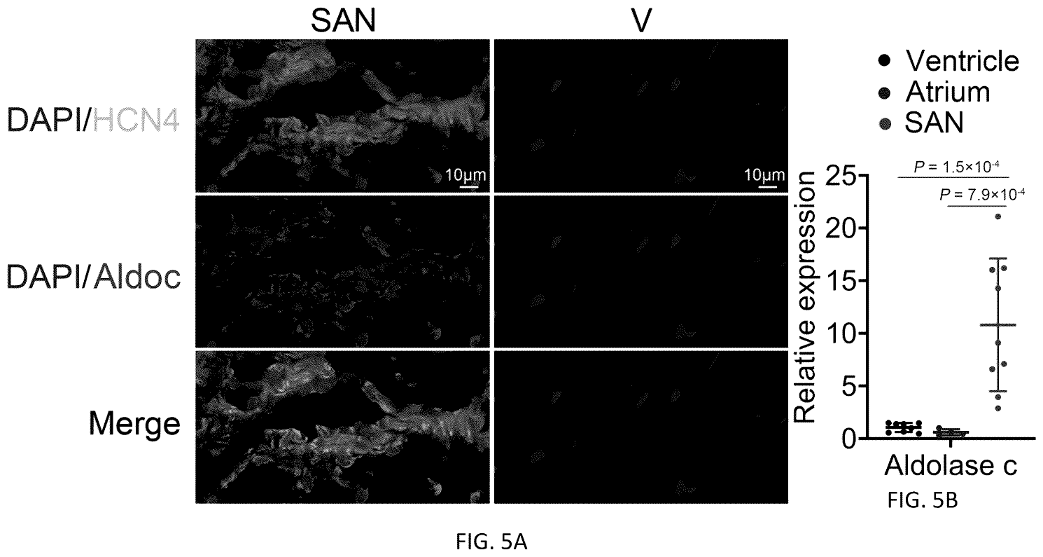

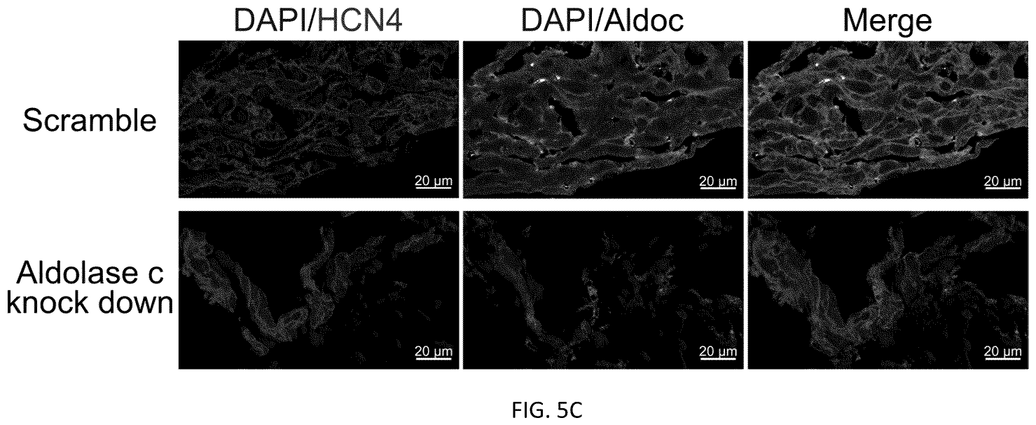

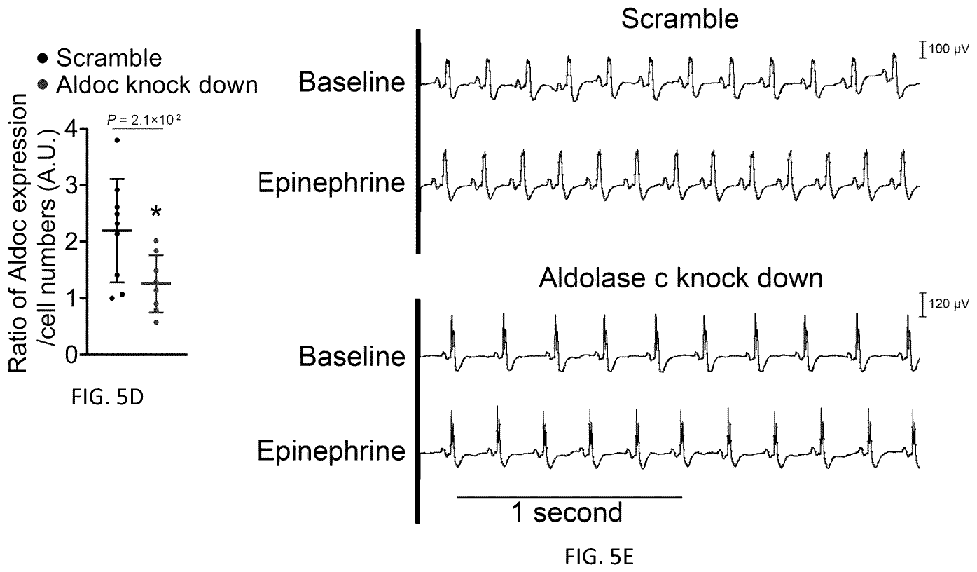

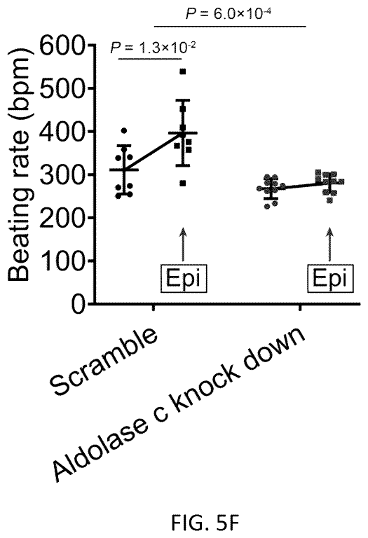

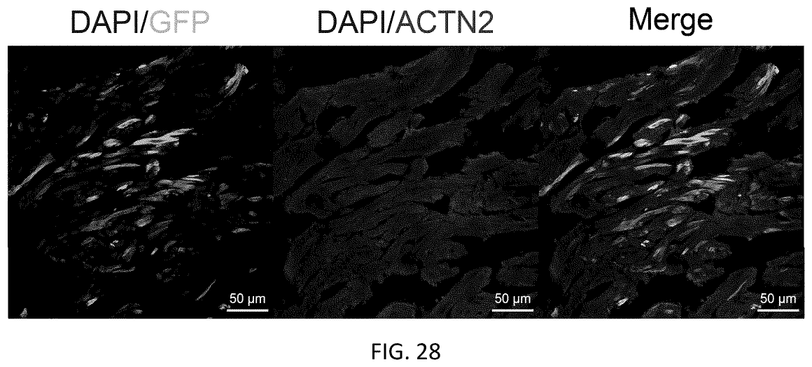

The regional distribution of Aldoc and its physiological function in the hearts of vertebrates were further explored. In rats and mice, through immunofluorescence staining, Aldoc expression was observed exclusively within SANs but not in the atrium or ventricle (FIG. 5A). This finding was consistent with the abundance of Aldoc transcripts in rat SANs, while Aldoc expression was almost undetectable in the atria and ventricles (FIG. 5B) (Linscheid et al., Nat Commun, 2019). In vivo knockdown of Aldoc was performed in mouse SANs to determine whether Aldoc regulates the pacemaker activity of SANs. The efficiency of AAV9 Aldoc siRNAs in reducing Aldoc expression was first confirmed via in vitro transduction of mouse cardiomyocytes (FIG. 27A-27B). Then, AAV9 Aldoc siRNAs were delivered into the pericardial recess around mouse SANs through a mini-thoracotomy and reached approximately 80% transduction efficiency (FIG. 28). The in vivo Aldoc expression in the mouse SAN was successfully reduced (FIGS. 5C and 5D). Mice that received Aldoc siRNAs had a lower spontaneous heart rate than those that received the scrambled controls (FIGS. 5E and F). In addition, the responses to epinephrine were nullified in mice that received Aldoc siRNAs (FIG. 5F). Accordingly, the expression of Aldoc within the SAN might not only drive glycolysis machinery but also regulate in vivo PC rhythmicity.

### Aldolase c Regulated Pacemaker Activity in Human Induced Pluripotent Stem Cell-Derived Cardiomyocytes

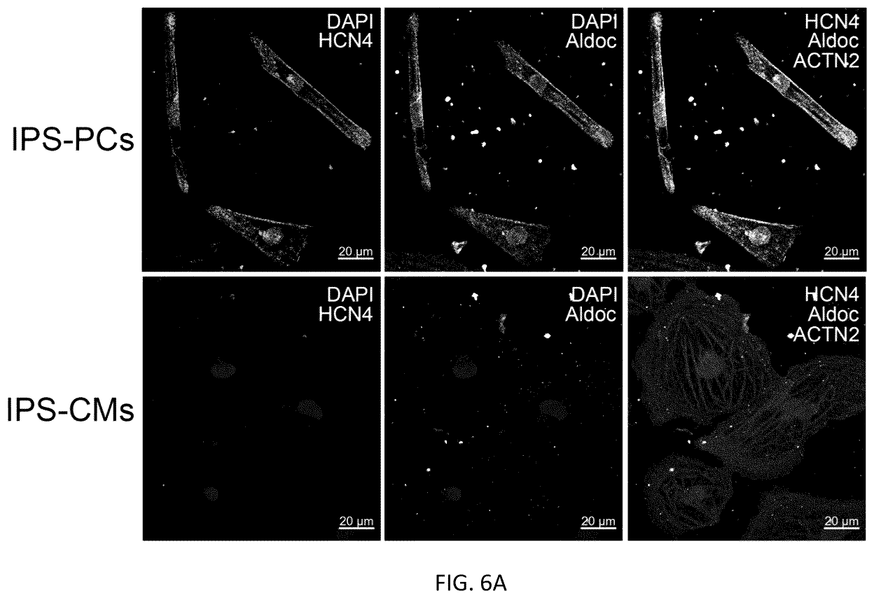

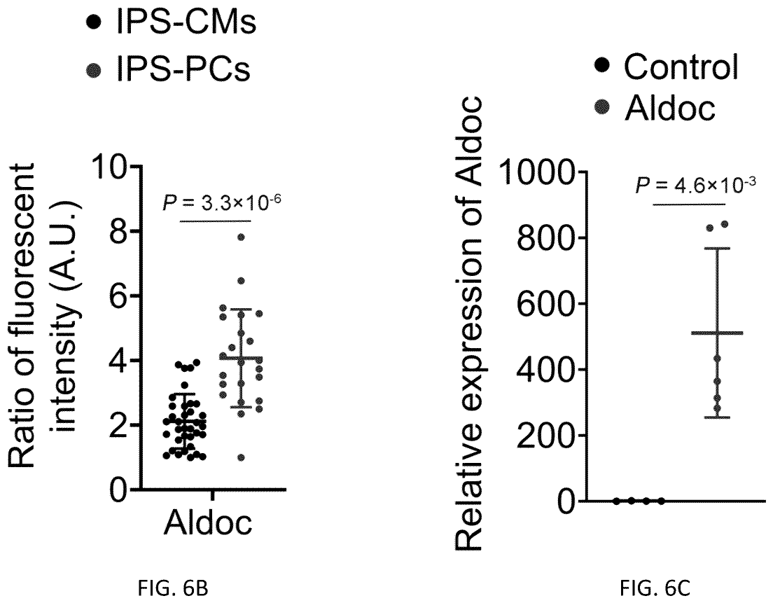

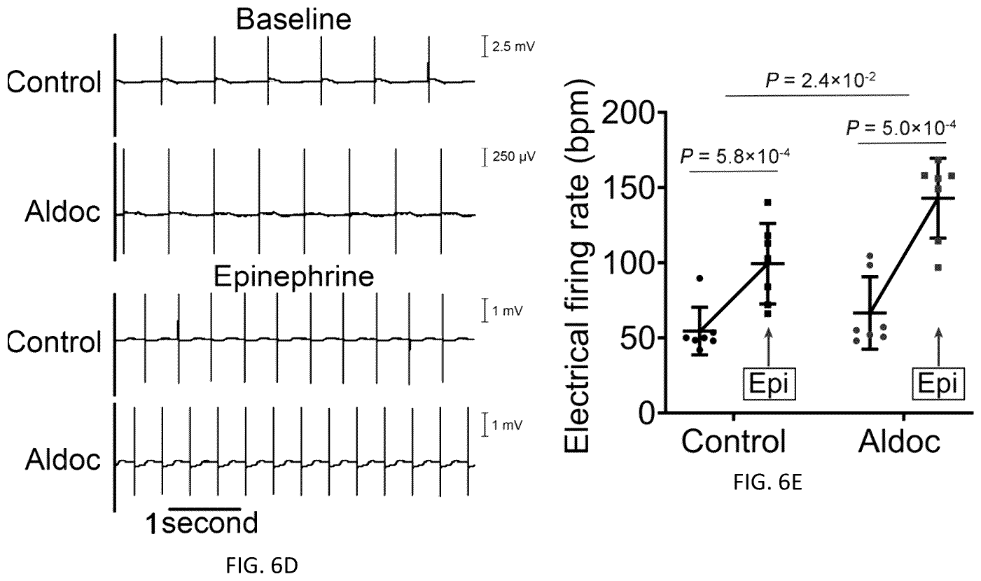

Induced pluripotent stem cell-derived cardiomyocytes (IPS-CMs) were used as a human cell model to study whether Aldoc regulates pacemaker activity in human PCs. HCN4 (+) PCs accounted for approximately 10% of IPS-CMs (Tsai et al., Stem Cell Res, 2021; Chang et al., Stem Cells, 2020), and were responsible for the rhythmic beating of IPS-CMs (Chiu et al., Stem Cell Res, 2021). Aldoc expression was predominantly observed in human HCN4 (+) PCs (FIGS. 6A and B). Overexpression of Aldoc in IPS-CMs via adenoviral vectors significantly increased Aldoc levels in IPS-CMs (FIG. 6C) and the electrical firing rate of IPS-CMs (FIGS. 6D and E). Human pacemaker cardiomyocytes share similar machinery in which Aldoc drives metabolic adaptation to regulate pacemaker rhythm, highlighting their translational potential for the study of human SAN physiology or disease prevention.

### Example 2 Inhibitors of PI3K Signal Pathway Increased Aldoc Expression and Beating Rates of Pacemaker Cardiomyocytes

In Example 1 above, integrin-dependent mitogen-activated protein kinase (MAPK)-E2F1 signal was found to turn on gene expression of Aldolase c in pacemaker cardiomyocytes. The PI3K pathway exerts its biological function through the activation of its down-streaming pathways including AKT and IKK. PI3K-AKT antagonizes the MAPK pathways and acts through direct inhibitory phosphorylation of Raf, a MAPK signaling component, by AKT, a key kinase acting downstream of PI3K (Hong et al., Circ Res, 2008). In pacemaker cardiomyocytes, the presence of the PI3K pathways is a counter-regulation of the MAPK pathway and therefore, inhibits the activation of aldolase c. From the analysis of whole transcriptome sequencing in the pacemaker cardiomyocytes from different cell models including Tbx18 or silk-fibroin-converted pacemaker cardiomyocytes, it was found that the activation of PI3K pathways is activated and the subtypes of PIK3K were mostly related to PI3K-α and PI3K-γ. These results suggested that the inhibition of the PI3K pathway in pacemaker cardiomyocytes might release the counter-regulation of MAPK and activate aldolase c, aerobic glycolysis, and increase beating rates in pacemaker cardiomyocytes. The in-vitro experiments using the inhibitor of PI3K (wortmannin, PIK-75, Duvelisib, Alpelisib, copanlisib, Idelalisib, and Eganelisib), and one IKK inhibitor (BMS-345541) all increased aldolase c expressions within pacemaker cardiomyocytes and increased beating rates. PI3K regulates aldolase c and glycolysis in pacemaker cardiomyocytes in a significant way. The drugs of specific inhibitors of PI3K (PIK-75, Duvelisib, Alpelisib, copanlisib, Idelalisib, and Eganelisib), and one IKK inhibitor (BMS-345541) are the target molecules for this example.

### Methods

### Neonatal Rat Ventricular Cardiomyocytes and Fibroblasts Isolation

Neonatal rat ventricular cardiomyocytes (VMs) were isolated from 1-2-day-old Sprague Dawley rat pups (laboratory animal center, National Yang Ming Chiao Tung University) as previously described (Kizana et al., Circ Res, 2007; Sekar et al., Circ Res, 2009). In brief, the lower one-third of the rat heart was cut to prevent atrioventricular nodal and His-Purkinje cells contamination. The VMs were isolated with 2.5% trypsin (15090046, no Phenol Red, Thermo Fisher Scientific [GIBCO®]), MA, USA) and collagenase (Type II, 17101015, GIBCO®), and resuspended in medium. After tissue lysis, the resuspended cells (fibroblasts and VMs) were seeded in a T150 culture flask (430824, Corning®, NY, USA) for 60 minutes (repeated twice) (Neuss et al., Cell Tissue Res, 1996). The nonadherent cells were mainly VMs, while attached cells were fibroblasts. The nonadherent cells were further seeded in 10% FBS medium for 2 days, and then the concentration of FBS (fetal bovine serum, SH30070.03, Cytiva [Hyclone], Washington, USA) in the medium was reduced to 2% for the experiment. The culture medium was based on M199 (11150059, Thermo Fisher Scientific) with the following components: 10 mM HEPES (14185052, Thermo Fisher Scientific), 0.1 mM non-essential amino acids (11140050, Thermo Fisher Scientific), 3.5 mg/mL glucose (G-7021, Sigma-Aldrich, MO, USA), 2 mM L-glutamine (A2916801, Thermo Fisher Scientific), 4 μg/ml vitamin B12 (V-2876, Sigma-Aldrich), 100 U/ml penicillin (15140122, Thermo Fisher Scientific) and FBS. For the collection of fibroblasts, 5 to 7 days until the confluence of the attached cells was reached, the fibroblasts were collected and stored with 10% dimethyl sulfoxide (DMSO/FBS, D2650, Sigma-Aldrich) in liquid nitrogen until use. Before the experiments of cells or engineered tissues, thawed fibroblasts were cultured until confluence, and then subcultures (1-2 passages) were used for the experiments.

### Whole-Transcriptome Analysis for Biomaterial-Converted Pacemaker Cardiomyocytes

The biomaterial-converted pacemaker cardiomyocytes were collected for RNA sequencing 2 d after seeding on the coated coverslip. The RNA library was constructed with the TruSeq RNA Library Preparation Kit v2 (Illumina). Briefly, mRNA was first purified and fragmented from 0.3 μg high-quality total RNA by oligo-dT attached magnetic beads. Purified mRNA was reverse-transcribed to double-stranded cDNA and further converted to blunt-end DNA by end repair. A tailing and ligation were performed to add the adapter. Finally, a few cycles of PCR were performed to enrich the library. The quality and quantity of the library were confirmed by gel electrophoresis, a Qubit HS DNA assay and qPCR measurement. A validated library was submitted for cluster generation and sequencing on the NextSeq 500 system (Illumina).

The following steps were used to analyse the results of the RNA sequencing. (1) Sequencing read quality control: FASTX-Toolkit (hannonlab.cshl.edu/fastx_toolkit) was employed to process the raw read data files. The command used was ‘fastq_quality_filter -Q33 -q 30 -p 70’. The command ‘-q 30’ indicates a minimum quality score of 30. The command ‘-p 70’ indicates that the minimum percent of bases must have ‘-q’ quality over or equal to 70%. (2) Alignment to the human genome and determination of gene expression: the ‘TopHat’ tool was used to align reads with the rat genome (genome version NCBI Rnor_6.0). Then, the ‘Cufflinks, version 2.2.1’ tool was used to calculate ‘FPKM’ for each gene and isoform, and test the statistical significance of differential changes, with expression adjustments made for multiple comparisons (Trapnell et al., Nat Protoc, 2012). Identification of differentially expressed genes (DGE): according to the expression level of genes from step 2 and the sample information, differentially expressed genes can be identified through the following criteria. First, the FPKM value should be >10 in case or control samples. Second, the log 2 ratio of the fold change is 1. Third, the P value should be <0.01 (Trapnell et al.). Pathway and gene ontology enrichment analysis: differentially expressed genes were applied to perform pathway and gene ontology enrichment analysis. The pathway information was extracted from NCBI BioSystems (ncbi.nlm.nih.gov/biosystems). The hypergeometric test was used to perform pathway enrichment analysis. The R package ‘GOstats’ command was used to perform gene ontology enrichment analysis. The pathways were also generated using the Ingenuity Target Explorer (IPA, Qiagen, targetexplorer.ingenuity.com/).

### Whole-Transcriptome Analysis for Tbx18-Converted Pacemaker Cardiomyocytes

The Tbx18-converted pacemaker cardiomyocytes were used for RNA sequencing 3-4 days after Tbx18 adenoviral transduction. RNA libraries were constructed by the TruSeq RNA Library Preparation Kits (Illumina) in accordance with the manufacturer's recommendations and previous literature (Yang et al., J Mol Cell Cardiol, 2012). Briefly, 3 μg of total RNA was first purified and fragmented by poly-T oligo-attached magnetic beads. The poly-A (+) RNA was reverse-transcribed to first double-stranded cDNA using random hexamers, converted to blunt-end DNA by end repair, and then adenylated (singly) at the 3′ ends. The cDNA samples were tailed and ligated by adding barcoded adapters. Individual cDNA libraries were enriched and purified. Five to six barcoded libraries were pooled in equimolar amounts (10 nmol/L) and diluted to 4 pmol/L, ensuring that clusters were formed in a single flow cell lane. Finally, single-end sequencing was performed with a NextSeq 500 (Illumina) sequencer. After removing the adapter sequence, data was demultiplexed and libraries were converted to FASTQ formations.

The alignment of the reading sequence to the rat genome was imported into HISAT2 (graph-based alignment of next generation sequencing reads to a population of genomes, (daehwankimlab.github.io/hisat2/)) reading sequence. Each transcriptome was normalized to the length of the individual transcriptome, and the total mapped read counts in each sample, and was expressed as RNA levels. The sequence data were mapped into different isoforms of individual genes and pooled together for subsequent comparative analysis. Imported gene symbols, sequences per million mapped reads, and fragments per kilobase per million (FPKM) values were imported into MultiExperiment Viewer (MeV v4.7.4) to compare mRNA expression values. The primary function of MultiExperiment Viewer includes computation of significant levels/false discovery rates, heat-map preparation, organizing tree analyses, and hierarchical clustering.

Differentially expressed genes were applied to perform pathway and gene ontology enrichment analysis. Pathway information was extracted from NCBI BioSystems (ncbi.nlm.nih.gov/biosystems). The hypergeometric test was used to perform pathway enrichment analysis. Gene ontology analyses were performed using the DAVID Bioinformatics Resources 6.8 database (david.ncifcrf.gov/). The pathways were also generated using the Ingenuity Target Explorer (IPA, Qiagen, targetexplorer.ingenuity.com/).

### Drug Treatment

The cocultures of Tbx18-pacemaker cardiomyocytes (PCs) and fibroblasts, a ratio of fibroblasts/VMs ( 1/10) was first plated on fibronectin-coated wells, and adenoviral Tbx18 (MOI [multiplicity of infection]: 10, ADV-225152, Vector Biolabs, Malvern, USA) was transduced for 24 hours after seeding to induce PCs. After re-expression of Tbx18 were treated with the inhibitors of PI3K (wortmannin, PIK-75, Duvelisib, Alpelisib, Copanlisib, Idelalisib, and Eganelisib), and one IKK inhibitor (BMS-345541) for 4 days. Then the beating rate and Aldoc transcriptional expressions were analyzed. The beating rates were recorded by video. An inverted microscope (AXIO Observer Al, Carl Zeiss AG, Oberkochen, Germany) was used for 10-second video capture. We analyzed the video with Q Capture Pro 6.0 (Teledyne Technologies, CA, USA). Aldoc gene transcriptional expressions were analyzed by quantitative real-time PCR. The protocol is as same as the treatment described previously (FIGS. 31A-31B).

### Quantitative Real-Time Polymerase Chain Reaction

Total RNA was extracted from cultured VMs using the RNeasy® Mini Kit (74106, Qiagen, Venlo, Netherlands). Complementary DNAs (cDNAs) were synthesized using a SuperScript® μl First-Strand Synthesis System (18080051, Thermo Fisher Science). Quantitative real-time PCR (qRT-PCR) was performed using Applied Biosystems™ StepOnePlus Real-Time PCR system with TaqMan probe assays (4331182, Invitrogen, Waltham, Massachusetts, USA) and PrimeTime qPCR Probe Assays (Integrated DNA Technologies, Inc., Coralville, Iowa, USA). The internal control was Gapdh. The conditions of thermocycle used in amplification were denaturation at 95° C. for 15 seconds and annealing and extension step at 60° C. for 60 seconds by 40 cycles. The relative values of gene transcripts were analyzed by ΔΔCt method.

### Results

The Transcriptional Regulation of PI3K in Induced Pacemaker Cardiomyocytes from Whole-Exome Sequences

### Gene Transcripts of PI3K in Biomaterial-Converted Pacemaker Cardiomyocytes

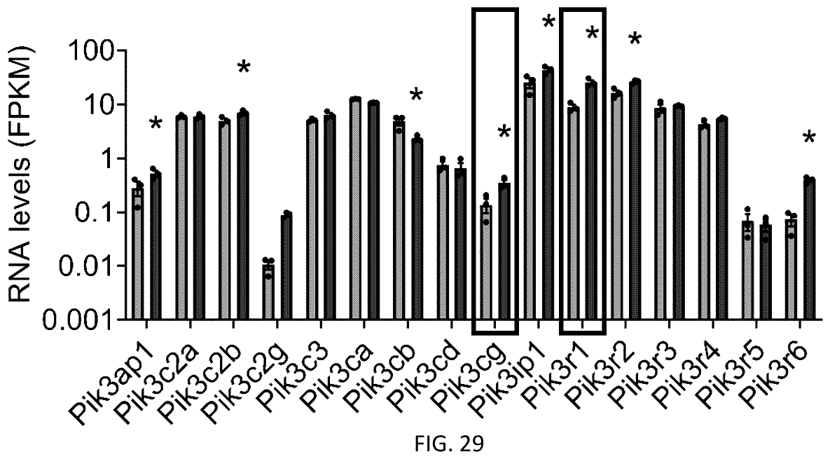

In the biomaterial-converted pacemaker cardiomyocytes, the gene transcripts related to PI3K-class I (Pik3r1[p85-α]), and class I (Pik3cg [p110-γ]) increased. Pik3r3 (p55-γ) showed a similar trend of increment, as compared to quiescent ventricular cardiomyocytes (FIG. 29).

### Gene Transcripts of PI3K in Tbx18-Converted Pacemaker Cardiomyocytes

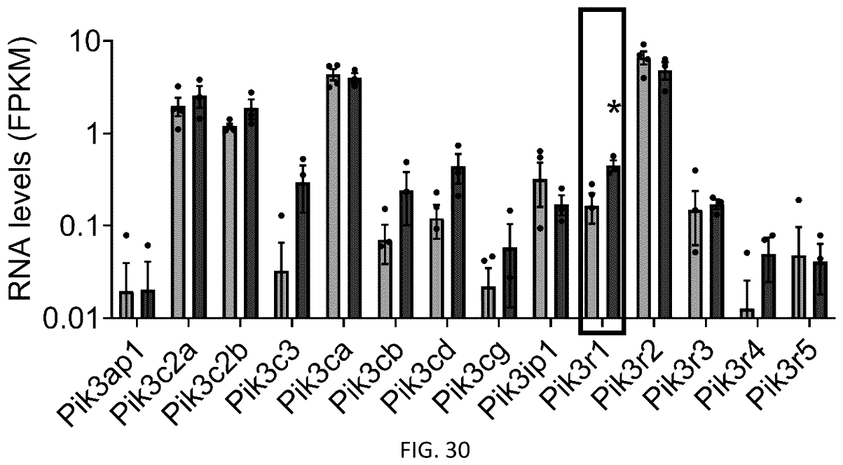

The gene transcripts related to PI3K-α (Pik3r1 [p85-α]) increased. PI3K-γ (Pik3cg[p110-γ] and Pik3r3[p55γ]) was a marginal increase, as compared to quiescent ventricular cardiomyocytes (FIG. 30).

### Increased Aldoc Expression and Beating Rate in Induced Pacemaker Cardiomyocytes by the Inhibitors of the PI3K-Pathway

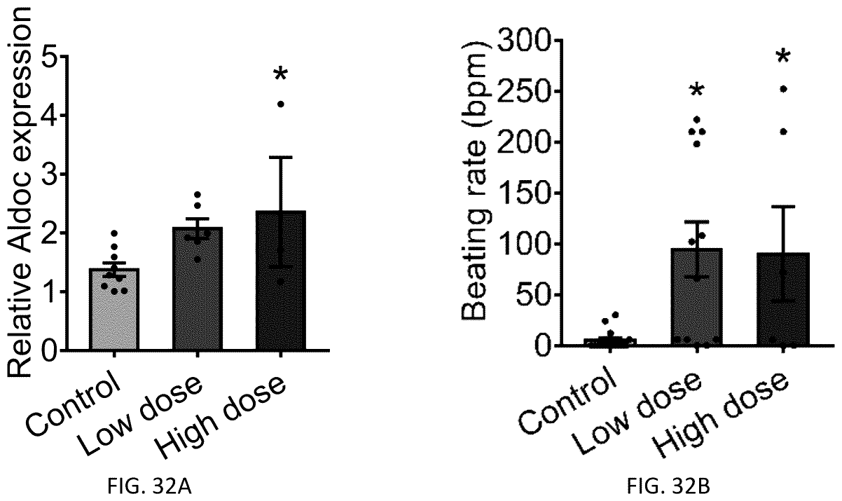

The Aldolase C expressions (P=0.049) and beating rate (P=0.017) were increased after the treatment of Wortmannin (100 nM) in pacemaker cardiomyocytes (FIGS. 31A-31B). The Aldolase C expressions increased in high dose (10 nM) and beating rate increased in both low (1-5 nM) and high dose (10 nM) after the treatment of PIK-75 in pacemaker cardiomyocytes (FIGS. 32A-32B).

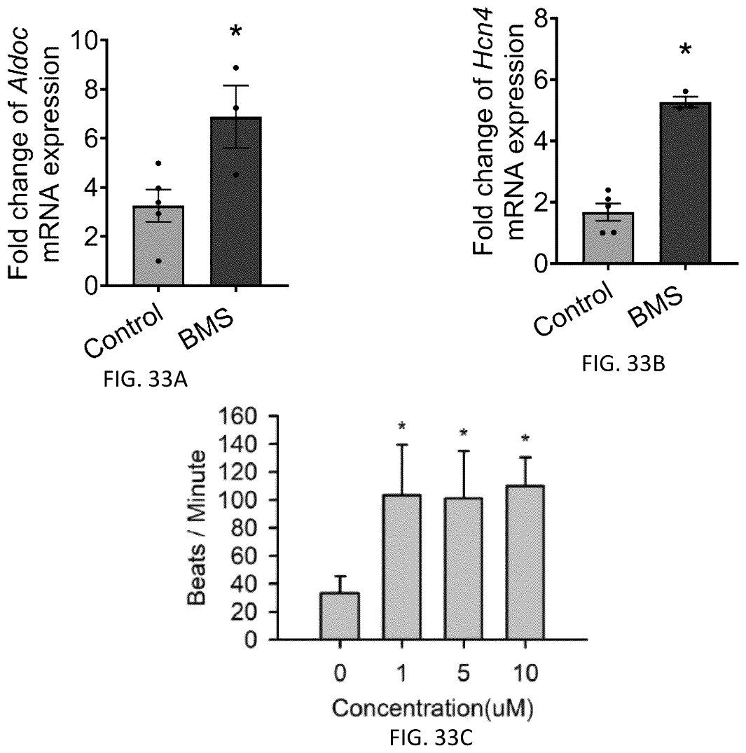

The Aldolase C and Hcn4 expressions were increased in 5 μM (*P<0.05 vs. control, by a 2-tailed t test), and beating rate increased in 1-10 μM (*P<0.05 vs. control, by one-way ANOVA) after the treatment of IKK inhibitor (BMS-345541) in pacemaker cardiomyocytes (FIGS. 33A-33C).

The transcriptional expressions of Aldolase C after the treatment of specific PI3K inhibitors were increased in control vs. Alpelisib (0.87±0.05 vs. 1.13±0.05, 250 nM, n=4-5, P=0.008), Idelalisib (0.87±0.05 vs. 1.01±0.02, 100 nM, n=5-6, P=0.02), Duvelisib (0.82±0.08 vs. 1.17±0.11, 50 nM, n=10-11, P=0.02), Copanlisib (0.64±0.09 vs. 1.32±0.09, 10 nM, n=4, P=0.002), and Eganelisib (0.57±0.03 vs. 0.82±0.07, 40 nM, n=5, P=0.013). All by P<0.05, by a 2-tailed t test.

The beating rate increased (1.6-fold, P=0.002) significantly after the treatment of these inhibitors by a 2-tailed t test.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of treating arrhythmia, comprising administering to a subject in need thereof an effective amount of a pharmaceutical composition including a therapeutic agent that increases the activity or expression of aldolase c (Aldoc) and a pharmaceutically acceptable carrier, the therapeutic agent modulating rhythmic activity of pacemaker cardiomyocytes in the subject.

2. The method as claimed in claim 1, wherein the therapeutic agent includes at least one selected from the group consisting of a phosphoinositide 3 kinase (PI3K) inhibitor, an IkappaB kinase (IKK) inhibitor, an integrin, a nucleic acid encoding an integrin, an integrin agonist, and a p38/MAPK agonist.

3. The method as claimed in claim 2, wherein the therapeutic agent includes the PI3K inhibitor.

4. The method as claimed in claim 3, wherein the PI3K inhibitor is selected from the group consisting of PIK-75, Duvelisib, Alpelisib, Copanlisib, Idelalisib, Eganelisib, and combinations thereof.

5. The method as claimed in claim 2, wherein the therapeutic agent includes the IKK inhibitor.

6. The method as claimed in claim 5, wherein the IKK inhibitor is BMS-345541.

7. The method as claimed in claim 1, wherein the subject is a human patient having or suspected of having the arrhythmia.

8. The method as claimed in claim 1, wherein the arrhythmia is selected from the group consisting of bradycardia arrhythmia, sick sinus syndrome (SSS), a sinoatrial node disease, a sinoatrial node dysfunction, and cardiac conduction disease.

9. The method as claimed in claim 8, wherein the arrhythmia is the cardiac conduction disease, which is atrioventricular block (AV block) or bundle block.

10. The method as claimed in claim 1, wherein the pharmaceutical composition is administered to the subject orally or via a parenteral route.

11. The method as claimed in claim 10, wherein the pharmaceutical composition is administered via the parenteral route, which is selected from the group consisting of intravenous injection, intraarterial injection, intraperitoneal injection, intrapleural injection, intracardiac injection, and intrapericardial injection.

12. A population of pacemaker cardiomyocytes, wherein the pacemaker cardiomyocytes are induced by T-box transcription factor 18 (Tbx18) and cultured in the presence of a therapeutic agent that increases the activity or expression of aldolase c (Aldoc) in the pacemaker cardiomyocytes.

13. The population of pacemaker cardiomyocytes as claimed in claim 12, wherein the therapeutic agent includes at least one selected from the group consisting of a phosphoinositide 3 kinase (PI3K) inhibitor, an IkappaB kinase (IKK) inhibitor, an integrin, a nucleic acid encoding an integrin, an integrin agonist, and a p38/MAPK agonist.

14. The population of pacemaker cardiomyocytes as claimed in claim 13, wherein the therapeutic agent includes the PI3K inhibitor.

15. The population of pacemaker cardiomyocytes as claimed in claim 14, wherein the PI3K inhibitor is selected from the group consisting of PIK-75, Duvelisib, Alpelisib, Copanlisib, Idelalisib, Eganelisib, and combinations thereof.

16. The population of pacemaker cardiomyocytes as claimed in claim 13, wherein the therapeutic agent includes the IKK inhibitor.

17. The population of pacemaker cardiomyocytes as claimed in claim 16, wherein the IKK inhibitor is BMS-345541.

18. A method of treating a cardiac arrhythmia, comprising administering to a subject in need thereof an effective amount of a population of pacemaker cardiomyocytes as claimed in claim 12.

19. The method as claimed in claim 18, wherein the cardiac arrhythmia is selected from the group consisting of bradycardia arrhythmia, sick sinus syndrome (SSS), a sinoatrial node disease, a sinoatrial node dysfunction, and cardiac conduction disease.

20. The method as claimed in claim 19, wherein the cardiac arrhythmia is the cardiac conduction disease, which is atrioventricular block (AV block) or bundle block.

21. The method as claimed in claim 18, wherein the population of pacemaker cardiomyocytes is administered by intravenous infusion or transplanted to a heart tissue of the subject.
